(19) United States
(12) Patent Application Publication
(10) Pub. No.: US 2026/0317428 A1
(43) Pub. Date: Oct. 15, 2026

(54) ANTI-FGFR2B ANTIBODIES AND USES THEREOF

(71) Applicant: 3H PHARMACEUTICALS CO., LTD., Shanghai (CN)

(72) Inventors: You LI, Shanghai (CN); Ruiqin WANG, Shanghai (CN); Jianwei ZHANG, Shanghai (CN); Shanshan ZHOU, Shanghai (CN); Xianqiang PING, Shanghai (CN); Jufang LIN, Shanghai (CN); Mingqing CAI, Shanghai (CN); Shaojing HU, Shanghai (CN)

(73) Assignee: 3H PHARMACEUTICALS CO., LTD., Shanghai (CN)

(21) Appl. No.: 19/116,647

(22) PCT Filed: Oct. 7, 2023
(86) PCT No.: PCT/CN2023/123142

§ 371 Date: Mar. 28, 2025

(30) Foreign Application Priority Data
Sep. 30, 2022 (WO) PCT/CN2022/122986

(51) Int. Cl. C07K 16/28; A61K 39/00; A61P 35/00

(52) U.S. Cl. CPC C07K 16/2863; A61P 35/00; A61K 2039/505; C07K 2317/24; C07K 2317/31; C07K 2317/33; C07K 2317/41; C07K 2317/565; C07K 2317/567; C07K 2317/732; C07K 2317/76; C07K 2317/92

(57) ABSTRACT

Novel anti-FGFR2b antibodies and antigen-binding fragments thereof specifically bind fibroblast growth factor receptor 2b (FGFR2IIIb, or FGFR2b), and polynucleotides, vectors, cells, compositions of the anti-FGFR2b.

### FIELD OF INVENTION

The present disclosure generally relates to novel anti-FGFR2b antibodies and antigen-binding fragment thereof, which specifically bind fibroblast growth factor receptor 2b (FGFR2IIIb, or FGFR2b), and polynucleotides, vectors, cells, compositions of the anti-FGFR2b, and uses thereof.

### CROSS REFERENCE TO RELATED APPLICATION

This application claims the priority to and benefits of International Application No. PCT/CN2022/122986, filed on Sep. 30 2022, which is incorporated herein by reference in its entirety.

### SEQUENCE LISTING

This disclosure contains a Sequence Listing electronically submitted as a file entitled “9.29.2023 sequence listing anti-FGFR2B.xml” having a size of 385,412 bytes and created on Sep. 29, 2023. The information contained in the Sequence Listing is incorporated by reference herein.

### BACKGROUND

Fibroblast growth factor receptors (FGFRs) pertain to a family of highly conserved transmembrane tyrosine kinase receptors consisting of an extracellular domain composed of different Ig-like domains (a isoform contains all three Ig-like domains D1, D2, and D3; β isoform contains only the two Ig-like domains D2 and D3 domains without D1), a transmembrane domain, and an intracellular tyrosine kinase domain. FGFRs are involved in various aspects of cancer biology, including cell proliferation, differentiation, migration, angiogenesis, and carcinogenesis (Katoh et al., Med Res Rev 2014; 34:280-300).

The D2 and D3 domains are critical for ligand binding and specificity, whereas the D1 domain, absent in certain isoforms, is thought to have an autoinhibitory function. Alternative splicing of the carboxyl half of the D3 domain yields either the IIIb or the IIIc isoform of FGFR1 to FGFR3 (Eswarakumar et al., Cytokine Growth Factor Rev 2005; 16:139-49; Turner et al., Nat Rev Cancer 2010; 10:116-29). These various isoforms of FGFRs exhibit tissue-specific expression and respond to a different spectrum of the 18 mammalian fibroblast growth factors (FGFs) (Beenken et al., Nat Rev Drug Discov 2009; 8:235-53). For FGFR2, alternative splicing of the exon encoding the D3 domain produces the FGFR2IIIb and FGFR2IIIc (also written as FGFR2b and FGFR2c) splice forms, which have distinct ligand-binding preferences. FGFR2IIIb, normally expressed on epithelial cells, is the specific receptor for the keratinocyte growth factor (KGF) family members (FGF10, FGF22, and especially FGF7), whereas FGFR2IIIc expressed on mesenchymal tissues responds to ligands secreted by epithelial cells, binds both FGF1 and FGF2 well, but does not bind the KGF family members (Ornitz et al., J Biol Chem 1996; 271:15292-7; Zhang et al., J Biol Chem 2006; 281:15694-700).

A wealth of evidence supports a critical role of aberrant FGFR2 signaling in cancer, including overexpression of FGFR2 and its ligands, mutations and amplifications of the receptor, and receptor isoform switching (Bai et al., Cancer Res; 70 (19): 7630-7639 Oct. 1, 2010). Single-nucleotide polymorphisms (SNP) of FGFR2 are associated with an increased risk of breast cancer development, presumably due to elevated FGFR2 expression (Easton et al., Nature 2007; 447:1087-93; Hunter et al., Nat Genet 2007; 39:870-4; Meyer et al., PLOS Biol 2008; 6: e108). Potentially activating missense mutations of FGFR2 have been reported in multiple cancer types, including endometrial, ovarian, breast, lung, and gastric cancers (Turner et al., Nat Rev Cancer 2010; 10:116-29; Davies et al., Cancer Res 2005; 65:7591-5; Ding et al., Nature 2008; 455:1069-75; Dutt et al., Proc Natl Acad Sci USA 2008; 105:8713-7; Greenman et al., Nature 2007; 446:153-8; Pollock et al., Oncogene 2007; 26:7158-62; Jang et al., Cancer Res 2001; 61:3541-3). Furthermore, the FGFR2 gene is amplified in a subset of gastric and breast cancers (Heiskanen et al., Anal Cell Pathol 2001; 22:229-34; Adnane et al., Oncogene 1991; 6:659-63; Turner et al., Oncogene 2010; 29:2013-23; Hara et al., Lab Invest 1998; 78:1143-53; Mor et al., Cancer Genet Cytogenet 1993; 65:111-4; Tsujimoto et al., Virchows Arch 1997; 431:383-9; Yoshida et al., Semin Cancer Biol 1993; 4:33-40). The coexpression of FGFR2IIIb and its ligand, FGF7, in pancreatic and gastric cancers, as well as in lung adenocarcinomas, is associated with poor prognosis (Yamayoshi et al., J Pathol 2004; 204:110-8; Cho et al., Am J Pathol 2007; 170:1964-74; Toyokawa et al., Oncol Rep 2009; 21:875-80), likely due to aberrant receptor activation through the formation of an autocrine activation loop. Paradoxically, FGFR2 has also been implicated as a tumor suppressor gene. For example, loss-of-function mutations have been detected in melanomas (Gartside et al., Mol Cancer Res 2009; 7:41-54). Decreased expression of FGFR2IIIb has been reported in several cancer types during tumor progression (Diez et al., Oncogene 1997; 14:323-30; Giri et al., Clin Cancer Res 1999; 5:1063-71; Ricol et al., Oncogene 1999; 18:7234-43; Zhang et al., Proc Natl Acad Sci USA 2001; 98:11336-40), possibly reflecting the physiologic role of FGFR2 in regulating tissue homeostasis (Grose et al., EMBO J 2007; 26:1268-78; Lin et al., Development 2007; 134:723-34). These contrasting and context dependent roles of FGFR2 signaling underscore the complexity of FGFR2 signaling.

Targeted therapy using anti-FGFR2b antibodies could be beneficial for patients with aberrantly activated/amplified FGFR2b signaling. There is a significant need for novel anti-FGFR2b antibodies.

### SUMMARY

The present disclosure provides novel anti-FGFR2b antibodies and antigen-binding fragment thereof, and polynucleotides, vectors, cells, compositions of the anti-FGFR2b, and uses thereof.

In an aspect, the present disclosure provides an antibody, comprising a heavy chain variable region, wherein the heavy chain variable region comprises at least one complementarity determining region (CDR), wherein the CDR that is farthest from the N terminus is selected from SEQ ID NOs: 61, 67, 73, 79, 85, 89, 95, 101, 104, 112, 116, 121, 125, 128, 131, 136, 138, 142, 144, 147, 151, 154, and 158.

In some embodiments, the heavy chain variable region comprises three CDRs, which are hCDR1, hCDR2, and hCDR3 from N terminus to C terminus, wherein the hCDR1 is selected from SEQ ID NOs: 59, 65, 71, 77, 83, 88, 93, 103, 110, 119, 135, 143, 146, 150, and 152; the hCDR2 is selected from SEQ ID NOs: 60, 66, 72, 78, 84, 94, 111, 118, 120, 124, 141, and 153; and the hCDR3 is selected from SEQ ID NOs: 61, 67, 73, 79, 85, 89, 95, 101, 104, 112, 116, 121, 125, 128, 131, 136, 138, 142, 144, 147, 151, 154, and 158.

In some embodiments, the hCDR1, hCDR2, and hCDR3 comprise:
a. SEQ ID NO: 59, SEQ ID NO: 60, SEQ ID NO: 61, respectively;
b. SEQ ID NO: 65, SEQ ID NO: 66, SEQ ID NO: 67, respectively;
c. SEQ ID NO: 71, SEQ ID NO: 72, SEQ ID NO: 73, respectively;
d. SEQ ID NO: 77, SEQ ID NO: 78, SEQ ID NO: 79, respectively;
e. SEQ ID NO: 83, SEQ ID NO: 84, SEQ ID NO: 85, respectively;
f. SEQ ID NO: 88, SEQ ID NO: 84, SEQ ID NO: 89, respectively;
g. SEQ ID NO: 93, SEQ ID NO: 94, SEQ ID NO: 95, respectively;
h. SEQ ID NO: 59, SEQ ID NO: 78, SEQ ID NO: 79, respectively;
i. SEQ ID NO: 93, SEQ ID NO: 94, SEQ ID NO: 101, respectively;
j. SEQ ID NO: 103, SEQ ID NO: 94, SEQ ID NO: 104, respectively;
k. SEQ ID NO: 59, SEQ ID NO: 78, SEQ ID NO: 79, respectively;
l. SEQ ID NO: 110, SEQ ID NO: 111, SEQ ID NO: 112, respectively;
m. SEQ ID NO: 88, SEQ ID NO: 84, SEQ ID NO: 116, respectively;
n. SEQ ID NO: 93, SEQ ID NO: 94, SEQ ID NO: 101, respectively;
o. SEQ ID NO: 93, SEQ ID NO: 118, SEQ ID NO: 101, respectively;
p. SEQ ID NO: 119, SEQ ID NO: 120, SEQ ID NO: 121, respectively;
q. SEQ ID NO: 119, SEQ ID NO: 124, SEQ ID NO: 125, respectively;
r. SEQ ID NO: 77, SEQ ID NO: 78, SEQ ID NO: 128, respectively;
s. SEQ ID NO: 83, SEQ ID NO: 94, SEQ ID NO: 131, respectively;
t. SEQ ID NO: 135, SEQ ID NO: 84, SEQ ID NO: 136, respectively;
u. SEQ ID NO: 88, SEQ ID NO: 84, SEQ ID NO: 138, respectively;
v. SEQ ID NO: 83, SEQ ID NO: 141, SEQ ID NO: 142, respectively;
w. SEQ ID NO: 143, SEQ ID NO: 84, SEQ ID NO: 144, respectively;
x. SEQ ID NO: 146, SEQ ID NO: 94, SEQ ID NO: 147, respectively;
y. SEQ ID NO: 119, SEQ ID NO: 120, SEQ ID NO: 121, respectively;
z. SEQ ID NO: 119, SEQ ID NO: 124, SEQ ID NO: 125, respectively;
aa. SEQ ID NO: 150, SEQ ID NO: 84, SEQ ID NO: 151, respectively;
bb. SEQ ID NO: 152, SEQ ID NO: 153, SEQ ID NO: 154, respectively; or
cc. SEQ ID NO: 88, SEQ ID NO: 94, SEQ ID NO: 158, respectively.

In some embodiments, the heavy chain variable region comprises four framework regions (FRs), which are hFR1, hFR2, hFR3, and hFR4 from N terminus to C terminus, wherein the hFR1 is selected from SEQ ID NOs: 160, 168, 175, 188, 194, 204, 210, 224, 233, 284, 259, 265, 268, 271, 274, 281, 295, and 305; the hFR2 is selected from SEQ ID NOs: 161, 169, 176, 183, 205, 221, 225, 237, 245, 251, 261, 266, 275, 282, and 296; the hFR3 is selected from SEQ ID NOs: 162, 170, 177, 184, 189, 195, 200, 206, 211, 216, 226, 231, 234, 239, 242, 246, 252, 254, 260, 262, 267, 272, 276, 297, and 306; and the hFR4 is selected from SEQ ID NOs: 163, 178, 190, 196, 212, 217, 227, 255, 269, and 298.

In some embodiments, the hFR1, hFR2, hFR3, and hFR4 comprise:
a. SEQ ID NO: 160, SEQ ID NO: 161, SEQ ID NO: 162, SEQ ID NO: 163, respectively;
b. SEQ ID NO: 168, SEQ ID NO: 169, SEQ ID NO: 170, SEQ ID NO: 163, respectively;
c. SEQ ID NO: 175, SEQ ID NO: 176, SEQ ID NO: 177, SEQ ID NO: 178, respectively;
d. SEQ ID NO: 160, SEQ ID NO: 183, SEQ ID NO: 184, SEQ ID NO: 178, respectively;
e. SEQ ID NO: 188, SEQ ID NO: 169, SEQ ID NO: 189, SEQ ID NO: 190, respectively;
f. SEQ ID NO: 194, SEQ ID NO: 169, SEQ ID NO: 195, SEQ ID NO: 196, respectively;
g. SEQ ID NO: 194, SEQ ID NO: 169, SEQ ID NO: 200, SEQ ID NO: 163, respectively;
h. SEQ ID NO: 204, SEQ ID NO: 205, SEQ ID NO: 206, SEQ ID NO: 178, respectively;
i. SEQ ID NO: 210, SEQ ID NO: 169, SEQ ID NO: 211, SEQ ID NO: 212, respectively;
j. SEQ ID NO: 194, SEQ ID NO: 169, SEQ ID NO: 216, SEQ ID NO: 217, respectively;
k. SEQ ID NO: 204, SEQ ID NO: 221, SEQ ID NO: 206, SEQ ID NO: 178, respectively;
l. SEQ ID NO: 224, SEQ ID NO: 225, SEQ ID NO: 226, SEQ ID NO: 227, respectively;
m. SEQ ID NO: 194, SEQ ID NO: 169, SEQ ID NO: 231, SEQ ID NO: 163, respectively;
n. SEQ ID NO: 233, SEQ ID NO: 169, SEQ ID NO: 234, SEQ ID NO: 178, respectively;
o. SEQ ID NO: 210, SEQ ID NO: 237, SEQ ID NO: 211, SEQ ID NO: 212, respectively;
p. SEQ ID NO: 194, SEQ ID NO: 169, SEQ ID NO: 239, SEQ ID NO: 190, respectively;
q. SEQ ID NO: 194, SEQ ID NO: 169, SEQ ID NO: 242, SEQ ID NO: 178, respectively;
r. SEQ ID NO: 284, SEQ ID NO: 183, SEQ ID NO: 184, SEQ ID NO: 163, respectively;
s. SEQ ID NO: 194, SEQ ID NO: 245, SEQ ID NO: 246, SEQ ID NO: 163, respectively;
t. SEQ ID NO: 188, SEQ ID NO: 251, SEQ ID NO: 252, SEQ ID NO: 163, respectively;
u. SEQ ID NO: 188, SEQ ID NO: 169, SEQ ID NO: 254, SEQ ID NO: 255, respectively;
v. SEQ ID NO: 259, SEQ ID NO: 169, SEQ ID NO: 260, SEQ ID NO: 190, respectively;
w. SEQ ID NO: 194, SEQ ID NO: 261, SEQ ID NO: 262, SEQ ID NO: 163, respectively;
x. SEQ ID NO: 265, SEQ ID NO: 266, SEQ ID NO: 267, SEQ ID NO: 163, respectively;
y. SEQ ID NO: 268, SEQ ID NO: 169, SEQ ID NO: 239, SEQ ID NO: 269, respectively;
z. SEQ ID NO: 194, SEQ ID NO: 169, SEQ ID NO: 200, SEQ ID NO: 190, respectively;
aa. SEQ ID NO: 271, SEQ ID NO: 169, SEQ ID NO: 272, SEQ ID NO: 190, respectively;
bb. SEQ ID NO: 274, SEQ ID NO: 275, SEQ ID NO: 276, SEQ ID NO: 178, respectively; or
cc. SEQ ID NO: 281, SEQ ID NO: 282, SEQ ID NO: 254, SEQ ID NO: 163, respectively,
dd. SEQ ID NO: 295, SEQ ID NO: 296, SEQ ID NO: 297, SEQ ID NO: 298, respectively;
ee. SEQ ID NO: 305, SEQ ID NO: 296, SEQ ID NO: 306, SEQ ID NO: 298, respectively;
ff. SEQ ID NO: 295, SEQ ID NO: 296, SEQ ID NO: 306, SEQ ID NO: 298, respectively;
gg. SEQ ID NO: 305, SEQ ID NO: 296, SEQ ID NO: 297, SEQ ID NO: 298, respectively;

In some embodiments, the CDRs and FRs in the heavy chain variable region are operably connected in the order of hFR1-hCDR1-hFR2-hCDR2-hFR3-hCDR3-hFR4 from N terminus to C terminus.

In some embodiments, the antibody further comprises a heavy chain constant region, wherein the heavy chain constant region comprises three domains, CH1, CH2, and CH3, from N-terminus to C terminus, and wherein the CH1, CH2, and CH3 are
SEQ ID NO. 307, SEQ ID NO. 309, and SEQ ID NO. 310, respectively; or
SEQ ID NO: 307, SEQ ID NO. 312, and SEQ ID NO. 313, respectively.

In some embodiments, the heavy chain variable region comprises an amino acid sequence with at least 80%, 85%, 90%, 95%, 96%, 97%, 98%, 99%, or 100% identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 285, 287, 290, and 291.

In some embodiments, the heavy chain variable region comprises an amino acid sequence of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 285, 287, 290, and 291.

In some embodiments, the antibody further comprises a light chain variable region, wherein the light chain variable region comprises at least one CDR, wherein the CDR that is farthest from the N terminus is selected from SEQ ID NOs: 64, 70, 76, 82, 87, 92, 98, 100, 102, 107, 109, 115, 117, 123, 127, 130, 134, 137, 140, 145, 157, and 159.

In some embodiments, the light chain variable region comprises three CDRs, which are 1CDR1, 1CDR2, and 1CDR3 from N terminus to C terminus, wherein the 1CDR1 is selected from SEQ ID NOs: 62, 68, 74, 80, 86, 90, 96, 99, 105, 108, 113, 122, 126, 129, 132, 148, 149, and 155; the 1CDR2 is selected from SEQ ID NOs: 63, 69, 75, 81, 91, 97, 106, 114, 133, 139, and 156; and the ICDR3 is selected from SEQ ID NOs: 64, 70, 76, 82, 87, 92, 98, 100, 102, 107, 109, 115, 117, 123, 127, 130, 134, 137, 140, 145, 157, and 159.

In some embodiments, the ICDR1, ICDR2, and ICDR3 comprise:
a. SEQ ID NO: 62, SEQ ID NO: 63, SEQ ID NO: 64, respectively;
b. SEQ ID NO: 68, SEQ ID NO: 69, SEQ ID NO: 70, respectively;
c. SEQ ID NO: 74, SEQ ID NO: 75, SEQ ID NO: 76, respectively;
d. SEQ ID NO: 80, SEQ ID NO: 81, SEQ ID NO: 82, respectively;
e. SEQ ID NO: 86, SEQ ID NO: 69, SEQ ID NO: 87, respectively;
f. SEQ ID NO: 90, SEQ ID NO: 91, SEQ ID NO: 92, respectively;
g. SEQ ID NO: 96, SEQ ID NO: 97, SEQ ID NO: 98, respectively;
h. SEQ ID NO: 99, SEQ ID NO: 69, SEQ ID NO: 100, respectively;
i. SEQ ID NO: 96, SEQ ID NO: 97, SEQ ID NO: 102, respectively;
j. SEQ ID NO: 105, SEQ ID NO: 106, SEQ ID NO: 107, respectively;
l. SEQ ID NO: 113, SEQ ID NO: 114, SEQ ID NO: 115, respectively;
m. SEQ ID NO: 105, SEQ ID NO: 106, SEQ ID NO: 117, respectively;
n. SEQ ID NO: 96, SEQ ID NO: 97, SEQ ID NO: 102, respectively;
o. SEQ ID NO: 96, SEQ ID NO: 97, SEQ ID NO: 102, respectively;
p. SEQ ID NO: 122, SEQ ID NO: 69, SEQ ID NO: 123, respectively;
q. SEQ ID NO: 126, SEQ ID NO: 69, SEQ ID NO: 127, respectively;
r. SEQ ID NO: 129, SEQ ID NO: 69, SEQ ID NO: 130, respectively;
s. SEQ ID NO: 132, SEQ ID NO: 133, SEQ ID NO: 134, respectively;
t. SEQ ID NO: 126, SEQ ID NO: 69, SEQ ID NO: 137, respectively; t.
u. SEQ ID NO: 132, SEQ ID NO: 139, SEQ ID NO: 140, respectively;
v. SEQ ID NO: 122, SEQ ID NO: 69, SEQ ID NO: 123, respectively;
w. SEQ ID NO: 90, SEQ ID NO: 91, SEQ ID NO: 145, respectively;
x. SEQ ID NO: 148, SEQ ID NO: 69, SEQ ID NO: 109, respectively;
y. SEQ ID NO: 149, SEQ ID NO: 63, SEQ ID NO: 109, respectively;
z. SEQ ID NO: 122, SEQ ID NO: 69, SEQ ID NO: 123, respectively;
aa. SEQ ID NO: 126, SEQ ID NO: 69, SEQ ID NO: 130, respectively;
bb. SEQ ID NO: 155, SEQ ID NO: 156, SEQ ID NO: 157, respectively; or
cc. SEQ ID NO: 155, SEQ ID NO: 156, SEQ ID NO: 159, respectively.

In some embodiments, the light chain variable region comprises four framework regions, which are IFR1, IFR2, IFR3, and IFR4 from N terminus to C terminus, wherein the IFR1 is selected from SEQ ID NOs: 164, 171, 179, 185, 191, 197, 201, 207, 213, 218, 222, 228, 232, 247, 256, 263, 270, 277, 283, 299, and 303; the IFR2 is selected from SEQ ID NOs: 165, 172, 180, 186, 192, 198, 202, 208, 214, 219, 223, 229, 235, 240, 243, 248, 257, 264, 278, 300, and 304; the IFR3 is selected from SEQ ID NOs: 166, 173, 181, 187, 193, 199, 203, 209, 220, 230, 236, 238, 241, 244, 249, 253, 258, 273, 279, and 301; and the 1FR4 is selected from SEQ ID NOs: 167, 174, 182, 215, 250, 280, and 302.

In some embodiments, the 1FR1, 1FR2, 1FR3, and IFR4 comprise:
a. SEQ ID NO: 164, SEQ ID NO: 165, SEQ ID NO: 166, SEQ ID NO: 167, respectively;
b. SEQ ID NO: 171, SEQ ID NO: 172, SEQ ID NO: 173, SEQ ID NO: 174, respectively;
c. SEQ ID NO: 179, SEQ ID NO: 180, SEQ ID NO: 181, SEQ ID NO: 182, respectively;
d. SEQ ID NO: 185, SEQ ID NO: 186, SEQ ID NO: 187, SEQ ID NO: 174, respectively;
e. SEQ ID NO: 191, SEQ ID NO: 192, SEQ ID NO: 193, SEQ ID NO: 174, respectively;
f. SEQ ID NO: 197, SEQ ID NO: 198, SEQ ID NO: 199, SEQ ID NO: 174, respectively;
g. SEQ ID NO: 201, SEQ ID NO: 202, SEQ ID NO: 203, SEQ ID NO: 167, respectively;
h. SEQ ID NO: 207, SEQ ID NO: 208, SEQ ID NO: 209, SEQ ID NO: 174, respectively;
i. SEQ ID NO: 213, SEQ ID NO: 214, SEQ ID NO: 203, SEQ ID NO: 215, respectively;
j. SEQ ID NO: 218, SEQ ID NO: 219, SEQ ID NO: 220, SEQ ID NO: 174, respectively;
k. SEQ ID NO: 222, SEQ ID NO: 223, SEQ ID NO: 209, SEQ ID NO: 174, respectively;
l. SEQ ID NO: 228, SEQ ID NO: 229, SEQ ID NO: 230, SEQ ID NO: 174, respectively;
m. SEQ ID NO: 232, SEQ ID NO: 219, SEQ ID NO: 220, SEQ ID NO: 167, respectively;
n. SEQ ID NO: 201, SEQ ID NO: 235, SEQ ID NO: 236, SEQ ID NO: 174, respectively;
o. SEQ ID NO: 201, SEQ ID NO: 214, SEQ ID NO: 238, SEQ ID NO: 215, respectively;
p. SEQ ID NO: 171, SEQ ID NO: 240, SEQ ID NO: 241, SEQ ID NO: 174, respectively;
q. SEQ ID NO: 171, SEQ ID NO: 243, SEQ ID NO: 244, SEQ ID NO: 174, respectively;
r. SEQ ID NO: 222, SEQ ID NO: 165, SEQ ID NO: 209, SEQ ID NO: 174, respectively;
s. SEQ ID NO: 247, SEQ ID NO: 248, SEQ ID NO: 249, SEQ ID NO: 250, respectively;
t. SEQ ID NO: 171, SEQ ID NO: 243, SEQ ID NO: 253, SEQ ID NO: 167, respectively;
u. SEQ ID NO: 256, SEQ ID NO: 257, SEQ ID NO: 258, SEQ ID NO: 174, respectively;
v. SEQ ID NO: 171, SEQ ID NO: 240, SEQ ID NO: 241, SEQ ID NO: 174, respectively;
w. SEQ ID NO: 263, SEQ ID NO: 264, SEQ ID NO: 199, SEQ ID NO: 174, respectively;
x. SEQ ID NO: 222, SEQ ID NO: 165, SEQ ID NO: 209, SEQ ID NO: 174, respectively;
y. SEQ ID NO: 270, SEQ ID NO: 165, SEQ ID NO: 166, SEQ ID NO: 174, respectively;
z. SEQ ID NO: 171, SEQ ID NO: 240, SEQ ID NO: 241, SEQ ID NO: 174, respectively;
aa. SEQ ID NO: 171, SEQ ID NO: 243, SEQ ID NO: 273, SEQ ID NO: 174, respectively;
bb. SEQ ID NO: 277, SEQ ID NO: 278, SEQ ID NO: 279, SEQ ID NO: 280, respectively; or
cc. SEQ ID NO: 283, SEQ ID NO: 278, SEQ ID NO: 279, SEQ ID NO: 174, respectively.
dd. SEQ ID NO: 299, SEQ ID NO: 300, SEQ ID NO: 301, SEQ ID NO: 302, respectively;
ee. SEQ ID NO: 303, SEQ ID NO: 304, SEQ ID NO: 301, SEQ ID NO: 302, respectively;
ff. SEQ ID NO: 299, SEQ ID NO: 304, SEQ ID NO: 301, SEQ ID NO: 302, respectively;

In some embodiments, the CDRs and FRs in the light chain variable region are operably connected in the order of IFR1-1CDR1-1FR2-1CDR2-1FR3-1CDR3-1FR4 from N terminus to C terminus.

In some embodiments, the antibody further comprises a light chain constant region (CL), wherein the CL is SEQ ID NO: 311.

In some embodiments, the light chain variable region comprises an amino acid sequence with at least 80%, 85%, 90%, 95%, 96%, 97%, 98%, 99%, or 100% identity to any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

In some embodiments, the light chain variable region comprises an amino acid sequence of any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

In another aspect, the present disclosure provides an antibody, comprising a light chain variable region, wherein the light chain variable region comprises at least one CDR, wherein the CDR that is farthest from the N terminus is selected from SEQ ID NOs: 64, 70, 76, 82, 87, 92, 98, 100, 102, 107, 109, 115, 117, 123, 127, 130, 134, 137, 140, 145, 157, and 159.

In some embodiments, the light chain variable region comprises three CDRs, which are 1CDR1, 1CDR2, and 1CDR3 from N terminus to C terminus, wherein the ICDR1 is selected from SEQ ID NOs: 62, 68, 74, 80, 86, 90, 96, 99, 105, 108, 113, 122, 126, 129, 132, 148, 149, and 155; the ICDR2 is selected from SEQ ID NOs: 63, 69, 75, 81, 91, 97, 106, 114, 133, 139, and 156; and the 1CDR3 is selected from SEQ ID NOs: 64, 70, 76, 82, 87, 92, 98, 100, 102, 107, 109, 115, 117, 123, 127, 130, 134, 137, 140, 145, 157, and 159.

In some embodiments, the ICDR1, 1CDR2, and ICDR3 comprise:
a. SEQ ID NO: 62, SEQ ID NO: 63, SEQ ID NO: 64, respectively;
b. SEQ ID NO: 68, SEQ ID NO: 69, SEQ ID NO: 70, respectively;
c. SEQ ID NO: 74, SEQ ID NO: 75, SEQ ID NO: 76, respectively;
d. SEQ ID NO: 80, SEQ ID NO: 81, SEQ ID NO: 82, respectively;
e. SEQ ID NO: 86, SEQ ID NO: 69, SEQ ID NO: 87, respectively;
f. SEQ ID NO: 90, SEQ ID NO: 91, SEQ ID NO: 92, respectively;
g. SEQ ID NO: 96, SEQ ID NO: 97, SEQ ID NO: 98, respectively;
h. SEQ ID NO: 99, SEQ ID NO: 69, SEQ ID NO: 100, respectively;
i. SEQ ID NO: 96, SEQ ID NO: 97, SEQ ID NO: 102, respectively;
j. SEQ ID NO: 105, SEQ ID NO: 106, SEQ ID NO: 107, respectively;
k. SEQ ID NO: 108, SEQ ID NO: 69, SEQ ID NO: 109, respectively;
l. SEQ ID NO: 113, SEQ ID NO: 114, SEQ ID NO: 115, respectively;
m. SEQ ID NO: 105, SEQ ID NO: 106, SEQ ID NO: 117, respectively;
n. SEQ ID NO: 96, SEQ ID NO: 97, SEQ ID NO: 102, respectively;
o. SEQ ID NO: 96, SEQ ID NO: 97, SEQ ID NO: 102, respectively;
p. SEQ ID NO: 122, SEQ ID NO: 69, SEQ ID NO: 123, respectively;
q. SEQ ID NO: 126, SEQ ID NO: 69, SEQ ID NO: 127, respectively;
r. SEQ ID NO: 129, SEQ ID NO: 69, SEQ ID NO: 130, respectively;
s. SEQ ID NO: 132, SEQ ID NO: 133, SEQ ID NO: 134, respectively;
t. SEQ ID NO: 126, SEQ ID NO: 69, SEQ ID NO: 137, respectively;
u. SEQ ID NO: 132, SEQ ID NO: 139, SEQ ID NO: 140, respectively;
v. SEQ ID NO: 122, SEQ ID NO: 69, SEQ ID NO: 123, respectively;
w. SEQ ID NO: 90, SEQ ID NO: 91, SEQ ID NO: 145, respectively;
x. SEQ ID NO: 148, SEQ ID NO: 69, SEQ ID NO: 109, respectively;
y. SEQ ID NO: 149, SEQ ID NO: 63, SEQ ID NO: 109, respectively;
z. SEQ ID NO: 122, SEQ ID NO: 69, SEQ ID NO: 123, respectively;
aa. SEQ ID NO: 126, SEQ ID NO: 69, SEQ ID NO: 130, respectively;
bb. SEQ ID NO: 155, SEQ ID NO: 156, SEQ ID NO: 157, respectively; or
cc. SEQ ID NO: 155, SEQ ID NO: 156, SEQ ID NO: 159, respectively.

In some embodiments, the light chain variable region comprises four framework regions, which are IFR1, IFR2, IFR3, and IFR4 from N terminus to C terminus, wherein the IFR1 is selected from SEQ ID NOs: 164, 171, 179, 185, 191, 197, 201, 207, 213, 218, 222, 228, 232, 247, 256, 263, 270, 277, 283, 299, and 303; the IFR2 is selected from SEQ ID NOs: 165, 172, 180, 186, 192, 198, 202, 208, 214, 219, 223, 229, 235, 240, 243, 248, 257, 264, 278, 300, and 304; the 1FR3 is selected from SEQ ID NOs: 166, 173, 181, 187, 193, 199, 203, 209, 220, 230, 236, 238, 241, 244, 249, 253, 258, 273, 279, and 301; and the IFR4 is selected from SEQ ID NOs: 167, 174, 182, 215, 250, 280, and 302.

In some embodiments, the 1FR1, 1FR2, 1FR3, and IFR4 comprise:
a. SEQ ID NO: 164, SEQ ID NO: 165, SEQ ID NO: 166, SEQ ID NO: 167, respectively;
b. SEQ ID NO: 171, SEQ ID NO: 172, SEQ ID NO: 173, SEQ ID NO: 174, respectively;
c. SEQ ID NO: 179, SEQ ID NO: 180, SEQ ID NO: 181, SEQ ID NO: 182,
d. SEQ ID NO: 185, SEQ ID NO: 186, SEQ ID NO: 187, SEQ ID NO: 174,
e. SEQ ID NO: 191, SEQ ID NO: 192, SEQ ID NO: 193, SEQ ID NO: 174, respectively;
f. SEQ ID NO: 197, SEQ ID NO: 198, SEQ ID NO: 199, SEQ ID NO: 174, respectively;
g. SEQ ID NO: 201, SEQ ID NO: 202, SEQ ID NO: 203, SEQ ID NO: 167, respectively;
h. SEQ ID NO: 207, SEQ ID NO: 208, SEQ ID NO: 209, SEQ ID NO: 174, respectively;
i. SEQ ID NO: 213, SEQ ID NO: 214, SEQ ID NO: 203, SEQ ID NO: 215, respectively;
j. SEQ ID NO: 218, SEQ ID NO: 219, SEQ ID NO: 220, SEQ ID NO: 174, respectively;
k. SEQ ID NO: 222, SEQ ID NO: 223, SEQ ID NO: 209, SEQ ID NO: 174, respectively;
l. SEQ ID NO: 228, SEQ ID NO: 229, SEQ ID NO: 230, SEQ ID NO: 174, respectively;
m. SEQ ID NO: 232, SEQ ID NO: 219, SEQ ID NO: 220, SEQ ID NO: 167, respectively;
n. SEQ ID NO: 201, SEQ ID NO: 235, SEQ ID NO: 236, SEQ ID NO: 174, respectively;
o. SEQ ID NO: 201, SEQ ID NO: 214, SEQ ID NO: 238, SEQ ID NO: 215, respectively;
p. SEQ ID NO: 171, SEQ ID NO: 240, SEQ ID NO: 241, SEQ ID NO: 174, respectively;
q. SEQ ID NO: 171, SEQ ID NO: 243, SEQ ID NO: 244, SEQ ID NO: 174, respectively;
r. SEQ ID NO: 222, SEQ ID NO: 165, SEQ ID NO: 209, SEQ ID NO: 174, respectively;
s. SEQ ID NO: 247, SEQ ID NO: 248, SEQ ID NO: 249, SEQ ID NO: 250, respectively;
t. SEQ ID NO: 171, SEQ ID NO: 243, SEQ ID NO: 253, SEQ ID NO: 167, respectively;
u. SEQ ID NO: 256, SEQ ID NO: 257, SEQ ID NO: 258, SEQ ID NO: 174, respectively;
v. SEQ ID NO: 171, SEQ ID NO: 240, SEQ ID NO: 241, SEQ ID NO: 174, respectively;
w. SEQ ID NO: 263, SEQ ID NO: 264, SEQ ID NO: 199, SEQ ID NO: 174, respectively;
x. SEQ ID NO: 222, SEQ ID NO: 165, SEQ ID NO: 209, SEQ ID NO: 174, respectively;
y. SEQ ID NO: 270, SEQ ID NO: 165, SEQ ID NO: 166, SEQ ID NO: 174, respectively;
z. SEQ ID NO: 171, SEQ ID NO: 240, SEQ ID NO: 241, SEQ ID NO: 174, respectively;
aa. SEQ ID NO: 171, SEQ ID NO: 243, SEQ ID NO: 273, SEQ ID NO: 174, respectively;
bb. SEQ ID NO: 277, SEQ ID NO: 278, SEQ ID NO: 279, SEQ ID NO: 280, respectively; or
cc. SEQ ID NO: 283, SEQ ID NO: 278, SEQ ID NO: 279, SEQ ID NO: 174, respectively,
dd. SEQ ID NO: 299, SEQ ID NO: 300, SEQ ID NO: 301, SEQ ID NO: 302, respectively;
ee. SEQ ID NO: 303, SEQ ID NO: 304, SEQ ID NO: 301, SEQ ID NO: 302, respectively;
ff. SEQ ID NO: 299, SEQ ID NO: 304, SEQ ID NO: 301, SEQ ID NO: 302, respectively;
gg.

In some embodiments, the CDRs and FRs in the light chain variable region are operably connected in the order of IFR1-ICDR1-IFR2-1CDR2-IFR3-ICDR3-IFR4 from N terminus to C terminus.

In some embodiments, the antibody further comprises a light chain constant region (CL), wherein the CL is SEQ ID NO: 311.

In some embodiments, the light chain variable region comprises an amino acid sequence with at least 80%, 85%, 90%, 95%, 96%, 97%, 98%, 99%, or 100% identity to any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

In some embodiments, the light chain variable region comprises an amino acid sequence of any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

In some embodiments, the heavy chain variable region and the light chain variable region comprise an amino acid sequence with at least 80%, 85%, 90%, 95%, 96%, 97%, 98%, 99%, or 100% identity to:
a. SEQ ID NO: 1, and SEQ ID NO: 2, respectively;
b. SEQ ID NO: 3, and SEQ ID NO: 4, respectively;
c. SEQ ID NO: 5, and SEQ ID NO: 6, respectively;
d. SEQ ID NO: 7, and SEQ ID NO: 8, respectively;
e. SEQ ID NO: 9, and SEQ ID NO: 10, respectively;
f. SEQ ID NO: 11, and SEQ ID NO: 12, respectively;
g. SEQ ID NO: 13, and SEQ ID NO: 14, respectively;
h. SEQ ID NO: 15, and SEQ ID NO: 16, respectively;
i. SEQ ID NO: 17, and SEQ ID NO: 18, respectively;
j. SEQ ID NO: 19, and SEQ ID NO: 20, respectively;
k. SEQ ID NO: 21, and SEQ ID NO: 22, respectively;
l. SEQ ID NO: 23, and SEQ ID NO: 24, respectively;
m. SEQ ID NO: 25, and SEQ ID NO: 26, respectively;
n. SEQ ID NO: 27, and SEQ ID NO: 28, respectively;
o. SEQ ID NO: 29, and SEQ ID NO: 30, respectively;
p. SEQ ID NO: 31, and SEQ ID NO: 32, respectively;
q. SEQ ID NO: 33, and SEQ ID NO: 34, respectively;
r. SEQ ID NO: 35, and SEQ ID NO: 36, respectively;
s. SEQ ID NO: 37, and SEQ ID NO: 38, respectively;
t. SEQ ID NO: 39, and SEQ ID NO: 40, respectively;
u. SEQ ID NO: 41, and SEQ ID NO: 42, respectively;
v. SEQ ID NO: 43, and SEQ ID NO: 32, respectively;
w. SEQ ID NO: 45, and SEQ ID NO: 46, respectively;
x. SEQ ID NO: 47, and SEQ ID NO: 48, respectively;
y. SEQ ID NO: 49, and SEQ ID NO: 50, respectively;
z. SEQ ID NO: 51, and SEQ ID NO: 32, respectively;
aa. SEQ ID NO: 53, and SEQ ID NO: 54, respectively;
bb. SEQ ID NO: 55, and SEQ ID NO: 56, respectively; or
cc. SEQ ID NO: 57, and SEQ ID NO: 58, respectively,
dd. SEQ ID NO: 285, and SEQ ID NO: 286, respectively;
ee. SEQ ID NO: 287, and SEQ ID NO: 288, respectively;
ff. SEQ ID NO: 287, and SEQ ID NO: 289, respectively;
gg. SEQ ID NO: 290, and SEQ ID NO: 288, respectively;
hh. SEQ ID NO: 290, and SEQ ID NO: 286, respectively;
ii. SEQ ID NO: 291, and SEQ ID NO: 286, respectively;
jj. SEQ ID NO: 292, and SEQ ID NO: 293, respectively;
kk. SEQ ID NO: 294, and SEQ ID NO: 293, respectively.

In some embodiments, the heavy chain variable region and the light chain variable region comprise:
a. SEQ ID NO: 1, and SEQ ID NO: 2, respectively;
b. SEQ ID NO: 3, and SEQ ID NO: 4, respectively;
c. SEQ ID NO: 5, and SEQ ID NO: 6, respectively;
d. SEQ ID NO: 7, and SEQ ID NO: 8, respectively;
e. SEQ ID NO: 9, and SEQ ID NO: 10, respectively;
f. SEQ ID NO: 11, and SEQ ID NO: 12, respectively;
g. SEQ ID NO: 13, and SEQ ID NO: 14, respectively;
h. SEQ ID NO: 15, and SEQ ID NO: 16, respectively;
i. SEQ ID NO: 17, and SEQ ID NO: 18, respectively;
j. SEQ ID NO: 19, and SEQ ID NO: 20, respectively;
k. SEQ ID NO: 21, and SEQ ID NO: 22, respectively;
l. SEQ ID NO: 23, and SEQ ID NO: 24, respectively;
m. SEQ ID NO: 25, and SEQ ID NO: 26, respectively;
n. SEQ ID NO: 27, and SEQ ID NO: 28, respectively;
o. SEQ ID NO: 29, and SEQ ID NO: 30, respectively;
p. SEQ ID NO: 31, and SEQ ID NO: 32, respectively;
q. SEQ ID NO: 33, and SEQ ID NO: 34, respectively;
r. SEQ ID NO: 35, and SEQ ID NO: 36, respectively;
s. SEQ ID NO: 37, and SEQ ID NO: 38, respectively;
t. SEQ ID NO: 39, and SEQ ID NO: 40, respectively; t.
u. SEQ ID NO: 41, and SEQ ID NO: 42, respectively; u.
v. SEQ ID NO: 43, and SEQ ID NO: 32, respectively;
w. SEQ ID NO: 45, and SEQ ID NO: 46, respectively;
x. SEQ ID NO: 47, and SEQ ID NO: 48, respectively;
y. SEQ ID NO: 49, and SEQ ID NO: 50, respectively;
z. SEQ ID NO: 51, and SEQ ID NO: 32, respectively;
aa. SEQ ID NO: 53, and SEQ ID NO: 54, respectively;
bb. SEQ ID NO: 55, and SEQ ID NO: 56, respectively; or
cc. SEQ ID NO: 57, and SEQ ID NO: 58, respectively.
dd. SEQ ID NO: 285, and SEQ ID NO: 286, respectively;
ee. SEQ ID NO: 287, and SEQ ID NO: 288, respectively;
ff. SEQ ID NO: 287, and SEQ ID NO: 289, respectively;
gg. SEQ ID NO: 290, and SEQ ID NO: 288, respectively;
hh. SEQ ID NO: 290, and SEQ ID NO: 286, respectively;
ii. SEQ ID NO: 291, and SEQ ID NO: 286, respectively;
jj. SEQ ID NO: 292, and SEQ ID NO: 293, respectively;
kk. SEQ ID NO: 294, and SEQ ID NO: 293, respectively.

In some embodiments, the antibody specifically binds fibroblast growth factor receptor 2b (FGFR2b).

In some embodiments, the FGFR2b is from an origin selected from human, mouse, and cynomolgus monkey.

In some embodiments, the antibody binds FGFR2b with a K_{D} of about 10 nM or lower.

In some embodiments, the antibody binds FGFR2b with a K_{D} of about 9 nM or lower.

In some embodiments, the antibody binds FGFR2b with a K_{D} of about 5 nM or lower.

In some embodiments, the antibody does not detectably bind to FGFR1b, FGFRIc, FGFR2c, FGFR3b, FGFR3c, or FGFR4.

In some embodiments, the antibody is a chimeric antibody.

In some embodiments, the antibody is a humanized or partially humanized antibody.

In some embodiments, the antibody is a monoclonal antibody.

In some embodiments, the antibody is an afucosylated antibody.

In some embodiments, the antibody is a Fc-engineered antibody, which enhanced binding to an activating Fc receptor, and enhances antibody-dependent cellular cytotoxicity (ADCC).

In some embodiments, the antibody is an ADCC-enhanced antibody.

In some embodiments, the antibody is a bispecific antibody.

In some embodiments, the antibody is operably linked to a cytotoxic agent.

In another aspect, the present disclosure provides a composition, comprising the antibody described herein.

In another aspect, the present disclosure provides a pharmaceutical composition, comprising the antibody described herein and a pharmaceutically acceptable carrier.

In another aspect, the present disclosure provides a polynucleotide encoding the antibody described herein.

In another aspect, the present disclosure provides a vector, comprising the polynucleotide described herein.

In another aspect, the present disclosure provides a cell capable of expressing the antibody described herein.

In another aspect, the present disclosure provides a cell comprising the polynucleotide described herein and/or the vector described herein.

In another aspect, the present disclosure provides a method of producing an antibody, comprising culturing the cell described herein and recovering the antibody from the cell.

In another aspect, the present disclosure provides a method for blocking FGFR2b from binding to at least one fibroblast growth factor (FGF), comprising administering to a subject an effective amount of the antibody described herein or the composition described herein.

In some embodiments, the FGF is selected from FGF1, FGF3, FGF7, FGF10, and FGF22.

In another aspect, the present disclosure provides a method for inhibiting cell proliferation, comprising administering to a subject an effective amount of the antibody described herein or the composition described herein.

In some embodiments, the cell proliferation is FGF-induced cell proliferation.

In some embodiments, the cell is a cancer cell.

In another aspect, the present disclosure provides a method for inhibiting the signal transduction pathway stimulated by binding of FGF to FGFR2b, comprising administering to a subject an effective amount of the antibody described herein or the composition described herein.

In another aspect, the present disclosure provides a method for inhibiting the growth of a tumor xenograft that overexpresses FGFR2b, comprising administering to a subject an effective amount of the antibody described herein or the composition described herein.

In another aspect, the present disclosure provides a method for detecting the presence of FGFR2b in a sample, comprising contacting the antibody described herein or the composition described herein with the sample, wherein a detection of at least one bound antibody indicates presence of FGFR2b.

### BRIEF DESCRIPTION OF THE FIGURES

FIG. 1 shows amino acid sequences of the entire mAb1 light chain (FIG. 1A) and heavy chain (FIG. 1B) with the CDRs underlined.

FIG. 2 shows BLI binding affinities of mAb1 to FGFR2b ECDs from 3 species with FPA144 as control antibody for reference comparison.

FIG. 3 shows results from flow cytometry of binding of the mAb1 and reference control FPA144 to FGFR2IIIb (FIG. 3A) and FGFR2IIIc (FIG. 3B) on FGFR2IIIb-HEK293 or FGFR2IIIc-HEK293 cells

FIG. 4 shows binding selectivity of the mAb1 to various family members of human FGFRs.

FIG. 5 shows inhibition level of FGF7-induced cell proliferation of SNU16 cells by mAb1 and reference control FPA144

FIG. 6 shows inhibition level of FGF10-induced cell proliferation of SNU16 cells by mAb1 and reference control FPA144.

FIG. 7 shows inhibition level of FGFR2 signaling pathway by mAb1 and reference control FPA144.

FIG. 8 shows in vivo antitumor efficacy of mAb1 at 5 mg/kg i.p. dosed twice a week in SNU16 gastric cancer xenograft model.

FIG. 9 shows amino acid sequences of the entire humAbA1 light chain (FIG. 9A) and heavy chain (FIG. 9B) with the CDRs underlined.

FIG. 10 shows BLI binding Ka, K_{off} (which is represented by K_{d} in the FIG. 10), and affinity KD of humAbA1 to FGFR2b extra-cellular domains (ECDs) from 3 species.

FIG. 11 shows results from flow cytometry of binding of the humAbA1 to FGFR2IIIb and FGFR2IIIc on FGFR2IIIb-HEK293 or FGFR2IIIc-HEK293 cells

FIG. 12 shows binding selectivity of the humAbA1 to various family members of human FGFRs.

FIG. 13 shows inhibition level of FGF7-induced cell proliferation of SNU16 cells by humAbA1 and reference control Bemarituzumab

FIG. 14 shows inhibition level of FGFR2 phosphorylation of SNU-16 cells.

FIG. 15 shows ADCC activity of antibodies and Bemarituzumab in CD16aV158 cell line (FIG. 15A), and in CD16aF158 cell line (FIG. 15B).

FIG. 16 shows in vivo antitumor efficacy of antibodies at 10 mg/kg intraperitoneal (i.p.) dosed twice a week in SNU-16 gastric cancer xenograft model.

FIG. 17 shows in vivo antitumor efficacy of antibodies at 10 mg/kg i.p. dosed twice a week in OCUM-2M gastric cancer xenograft model.

### DETAILED DESCRIPTION

### Definitions

All publications, patents, and patent applications referred to herein are incorporated by reference in their entirety to the same extent as if each individual publication, patent, or patent application was specifically and individually indicated to be incorporated by reference in its entirety.

In the present disclosure, unless otherwise specified, the scientific and technical terms used herein have the meanings generally understood by a person skilled in the art. Although any methods and materials similar or equivalent to those described herein find use in the practice of the present disclosure, the preferred methods and materials are described herein. Accordingly, the terms defined herein are more fully described by reference to the Specification as a whole.

As used herein, the singular terms “a,” “an,” and “the” include the plural reference unless the context clearly indicates otherwise.

As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

The term “about,” as used herein when referring to a measurable value such as a sequence length and the like, is meant to encompass variations of 5%, 1%, 0.5%, or even 0.1% of the specified amount.

Unless the context requires otherwise, the terms “comprise,” “comprises,” and “comprising,” or similar terms are intended to mean a non-exclusive inclusion, such that a recited list of elements or features does not include those stated or listed elements solely, but may include other elements or features that are not listed or stated.

Unless otherwise indicated, nucleic acids are written left to right in the 5′ to 3′ orientation; and amino acid sequences are written left to right in amino to carboxy orientation, respectively.

It is to be understood that this disclosure is not limited to the particular methodology, protocols, and reagents described, as these may vary, depending upon the context in which they are used by those skilled in the art.

As used herein, the terms “percent identity” and “% identity,” as applied to nucleic acid or polynucleotide sequences, refer to the percentage of residue matches between at least two nucleic acid or polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between nucleic acid or polynucleotide sequences may be determined using a suite of commonly used and freely available sequence comparison algorithms provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, MD, and on the Internet at http://www.ncbi.nlm.nih.gov/BLAST/.

Nucleic acid or polynucleotide sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein. Specifically, degenerate codon substitutions may be achieved by generating sequences in which the third position of one or more selected (or all) codons is substituted with mixed-base and/or deoxyinosine residues (Batzer et al. (1991) Nucleic Acid Res 19:5081; Ohtsuka et al. (1985) J Biol Chem 260:2605-2608; Rossolini et al. (1994) Mol Cell Probes 8:91-98).

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. The term nucleic acid is used interchangeably with polynucleotide, and (in appropriate contexts) gene, cDNA, and mRNA encoded by a gene.

As used herein, “percent (%) amino acid sequence identity” with respect to a peptide, polypeptide or protein sequence is defined as the percentage of amino acid residues in a candidate sequence that are identical with the amino acid residues in another peptide or polypeptide sequence, after aligning the sequences and introducing gaps, if necessary, to achieve the maximum percent sequence identity, and not considering any conservative substitutions as part of the sequence identity. Percent amino acid sequence identity in the current disclosure is measured using BLAST software. Those skilled in the art can determine appropriate parameters for measuring alignment, including any algorithms needed to achieve maximal alignment over the full length of the sequences being compared.

An amino acid substitution refers to the replacement of one amino acid in a polypeptide with another amino acid. Exemplary substitutions are shown in Table 1. Amino acid substitutions may be introduced into a protein of interest and the products screened for a desired activity, for example, retained/improved biological activity.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. The term, “corresponding to” with reference to nucleotide or amino acid positions of a sequence, such as set forth in the Sequence Listing, refers to nucleotides or amino acid positions identified upon alignment with a target sequence based on structural sequence alignment or using a standard alignment algorithm, such as the GAP algorithm. For example, corresponding residues of a similar sequence (e.g., a fragment or species variant) can be determined by alignment to a reference sequence by structural alignment methods. By aligning the sequences, one skilled in the art can identify corresponding residues, for example, using conserved and identical amino acid residues as guides.

As used herein, a composition refers to any mixture of two or more products, substances, or compounds, including cells.

As used herein, a “pharmaceutical composition” refers to an active pharmaceutical agent formulated in pharmaceutically acceptable or physiologically acceptable solutions for administration to a cell or an animal, either alone, or in combination with one or more other modalities of therapy. It will also be understood that, if desired, the compositions of the disclosure may be administered in combination with other agents, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy. Some non-limiting examples of the components that could be included in the composition are carriers, stabilizers, diluents, dispersing agents, suspending agents, thickening agents, and/or excipients. The pharmaceutical composition facilitates administration of the antibody or cell described herein to a subject. Multiple techniques of administration exist in the art including, but not limited to, intravenous, oral, aerosol, parenteral, ophthalmic, pulmonary, and topical administration.

As used herein, the term “pharmaceutically acceptable” refers to a material, such as a carrier or diluent, which does not abrogate the biological activity or properties of a therapeutic compound, and is relatively nontoxic, i.e., the material may be administered to a subject without causing undesirable biological effects or interacting in a deleterious manner with any of the components of the composition in which it is contained. Pharmaceutically acceptable components include those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, an “effective amount” refers to an amount of a pharmaceutical composition which is sufficient to significantly and positively modify the symptoms and/or conditions to be treated (e.g., provide a positive clinical response). The effective amount of a pharmaceutical composition will vary with the particular condition being treated, the severity of the condition, the duration of treatment, the nature of concurrent therapy, the particular composition being employed, the particular pharmaceutically-acceptable excipient(s) and/or carrier(s) utilized, and like factors with the knowledge and expertise of the attending physician.

As used herein, the terms “individual” and “subject” are used interchangeably herein to refer to an animal. For example, in some embodiments, the animal is a mammal. In some embodiments, the animals are humans, rodents, simians, felines, canines, equines, bovines, porcines, ovines, caprines, mammalian laboratory animals, mammalian farm animals, mammalian sport animals, or mammalian pets. The animal can be male or female and can be at any suitable age, including infant, juvenile, adolescent, adult, and geriatric. In some examples, an “individual” or “subject” refers to an animal in need of treatment for a disease or disorder. In some embodiments, the animal to receive the treatment can be a “patient,” designating the fact that the animal has been identified as having a disorder of relevance to the treatment, or being at adequate risk of contracting the disorder. In particular embodiments, the animal is a human, such as a human patient.

As used herein, “epitope” refers to the part of an antigen that is recognized and bound by an antibody. An antigen may have more than one epitopes that are recognized by an antibody. Epitopes often consist of a chemically active surface grouping of molecules such as amino acids or sugar side chains and have specific three-dimensional structural characteristics as well as specific charge characteristics.

As used herein, the term “FGFR2IIIb” and “FGFR2b” are used interchangeably to refer to the subtype IIIb splice form of FGFR2. Exemplary sequences of FGFR2b include Homo sapiens (human) FGFR2b protein (e.g., precursor sequence with signal peptide, Genbank accession number: NP 075259.4); Mus musculus (mouse) FGFR2b protein (e.g., full sequence, Genbank accession number: NP_963895.2).

As used herein, the term “FGFR2IIIc” and “FGFR2c” are used interchangeably to refer to the subtype IIIc splice form of FGFR2. Exemplary sequences of FGFR2c include human FGFR2c protein (e.g., precursor sequence, Genbank accession number: NP 000132.3).

As used herein, the term “anti-FGFR2b antibody” refers to an antibody that is capable of specifically binding to FGFR2b. In some embodiments, the anti-FGFR2b antibodies provided herein are specifically binding to both FGFR2b, but do not have detectable binding affinity to FGFR1b, FGFR1c, FGFR2c, FGFR3b, FGFR3c or FGFR4.

As used herein, the terms “specifically bind” or “bind” to a particular antigen refer to binding that is measurably different from a non-specific interaction. For example, in some embodiments, a binding molecule, such as an antibody, specifically binds to a target molecule, such as an antigen, when the binding molecule reacts or associates more frequently, more rapidly, with greater duration, and/or with greater affinity with the particular target molecule than it does with alternative molecules. A binding molecule, such as an antibody, “specifically binds” to a target molecule if it binds with greater affinity, avidity, more readily, and/or with greater duration than it binds to other molecules. It is understood that a binding molecule, such as an antibody, that specifically binds to a first target may or may not specifically bind to a second target. As such, “specific binding” does not necessarily require (although it can include) exclusive binding. In some embodiments, specific binding can be determined, for example, by comparing binding of a particular antibody to binding of an antibody that does not bind to a particular antigen. Specific binding for a particular antigen can be shown, for example, when an antibody has a K_{D} for an antigen of at most about 10⁻⁴ M, at most about 10⁻⁵ M, at most about 10⁻⁶ M, at least about 10⁻⁷ M, at most about 10⁻⁸ M, at most about 10⁻⁹ M, where Kp refers to a dissociation rate of the antibody/antigen interaction. In some embodiments, an antibody that specifically binds an antigen will have a Kp that is 20, 50, 100, 500, 1000, 5,000, 10,000 or more times smaller than the Kp of an antibody that does not bind to the same antigen. In some embodiments, the binding between an antibody and a particular antigen can be shown by an EC50 value, determined using suitable methods known in the art, including, for example, flow cytometry assay. Specific binding is a non-random binding reaction between two molecules, such as for example between an antibody and an antigen. Binding affinity of the antibody and antigen-binding fragment provided herein can be represented by K_{D} value, which represents the ratio of dissociation rate to association rate (K_{off}/Kon) when the binding between the antigen and antigen-binding molecule (e.g., the antibody and antigen-binding fragment) reaches equilibrium. The antigen-binding affinity (e.g., K_{D}) can be appropriately determined using suitable methods known in the art, including, for example, Biacore (which is based on surface plasmon resonance technology), and the Octet® BLI Label-Free Detection Systems.

As used herein, “affinity” refers to the strength of the sum total of noncovalent interactions between a single binding site of a molecule (e.g., a receptor) and its binding partner (e.g., a ligand). The affinity of a molecule for its partner can generally be represented by the equilibrium dissociation constant (K_{D}) (or its inverse equilibrium association constant, KA). Affinity can be measured by common methods known in the art, including those described herein (See e.g., Pope et al., J. Immunol. Methods. 2009; 341 (1-2): 86-96).

### Antibodies

Antibodies are widely used for biotherapeutics. The potential and use of antibodies as therapeutics for a wide range of diseases is due to the high specificity and affinity of their binding, facilitated through the variability in their complementarity determining regions (CDRs) (Liu J. K. H. Ann. Med. Surg., 3, 113-116 (2014)).

An “antibody” refers to a glycoprotein comprising at least two heavy (H) chains and two light (L) chains inter-connected by disulfide bonds, or an antigen binding fragment thereof. An “antibody” also refers to an IgA, IgD, IgE, IgG, or IgM antibody subtype, or an antigen binding fragment thereof. Each heavy chain is comprised of a heavy chain variable region (VH) and a heavy chain constant region (CH). The heavy chain constant region is typically comprised of three domains, CH1, CH2 and CH3. Each light chain is comprised of a light chain variable region (VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus (N-terminus) to carboxy-terminus (C-terminus) in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors. The six CDRs in an antibody variable domain fold up together in 3-D space to form the actual antibody binding site which locks onto the target antigen (Chothia et al., J Mol Biol, 1978;196, 901-17; Chothia et al., Nature, 1989; 342 (6252), 877-883; Kabat et al., Sequences of Proteins of Immunological Interest. 5th edn. National Institutes of Health Publication No. 91-3242. National Institutes of Health, Bethesda, MD (1991); Al-Lazikani et al., J. Mol. Bio., 1997; 273 (4), 927-948; Lefranc er al., Developmental & Comparative Immunology, 2003; 27 (1), 55-77).

A “CDR” of a variable domain are amino acid residues within the variable region that are identified in accordance with any CDR determination methods and/or definitions known in the art, including, but not limited to, Kabat definition, Chothia definition, the accumulation of both Kabat and Chothia definitions, AbM definition, contact definition, and conformational definition. Antibody CDRs may be identified as the hypervariable regions originally defined in Kabat et al., Sequences of Proteins of Immunological Interest. (1991) 5th edn. National Institutes of Health Publication No. 91-3242. National Institutes of Health, Bethesda, MD. The positions of CDRs may also be identified as the structural loop structures as described in Chothia et al., Nature 342.6252 (1989): 877-883. Other approaches to CDR identification include the “IMGT definition” (Lefranc, M.-P. et al., 1999, Nucleic Acids Res. 27:209-212); the “AbM definition,” which is a compromise between Kabat and Chothia and is derived using Oxford Molecular's AbM antibody modeling software; and the “contact definition” of CDRs based on observed antigen contacts, set forth in MacCallum et al., 1996, J. Mol. Biol. 262:732-745. In another approach, referred to herein as the “conformational definition” of CDRs, the positions of the CDRs may be identified as the residues that make enthalpic contributions to antigen binding (Makabe et al., 2008, J. Biol. Chem. 283:1156-1166). As used herein, a CDR refers to CDRs defined by any approach known in the art, including combinations of approaches.

The term “antigen-binding portion” or “antigen-binding fragment” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include, but are not limited to, (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a single VH domain or a single VL domain (see Ward et al., 1989, Nature 341:544-546); and (vi) an isolated complementarity determining region. Furthermore, although the two domains of the Fv fragment, VL and VH, are coded by separate genes, they can be joined, using recombinant methods, by a synthetic linker that enables them to be made as a single protein chain in which the VL and VH regions pair forms monovalent molecules, known as single chain Fv (scFv) (see e.g., Bird et al., 1988, Science 242:423-426; Huston et al., 1988, Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

For the purpose of this disclosure, the term “antibody” includes whole antibodies and any antigen binding fragment or single chains thereof. The term “antibody” includes, but are not limited to, monoclonal antibody, polyclonal antibody, chimeric antibody, recombinant antibody, single-chain variable fragment (ScFv), heavy-chain antibody, single domain antibody, humanized antibody, human antibody, and antibody from other origins such as mouse and rabbits.

As used herein, “isotype” refers to the class or subtype of antibody (e.g., IgA, IgD, IgE, IgG and IgM) which is determined by the heavy chain constant domains. In humans and most mammals, an antibody unit typically consists of four polypeptide chains: two identical heavy chains and two identical light chains connected by disulfide bonds. Light chains consist of one variable domain VL and one constant domain CL, while heavy chains contain one variable domain VH and three to four constant domains, e.g., CH1, CH2, CH3. Immunoglobulins can be assigned to five major classes, namely IgA, IgD, IgE, IgG, and IgM, depending on the heavy chain constant domain amino acid sequence. IgA and IgG are typically further sub-classified to IgA1, IgA2, IgG1, IgG2, IgG3, and IgG4. Antibody light chains of vertebrate species can be assigned to one of two distinct types, namely kappa (K and lambda (2), based on the amino acid sequences of their constant domains.

As used herein, the term “antigen” has the same meaning as “immunogen”.

As used herein, the term “Fc” refers to the portion of an antibody consisting of the second and third constant regions of a first heavy chain bound to the second and third constant regions of a second heavy chain via disulfide bonding. The Fc portion of the antibody is responsible for various effector functions such as antibody-dependent cell-mediated cytotoxicity (ADCC), and complement dependent cytotoxicity (CDC), but does not function in antigen binding.

In an aspect, the present disclosure provides an antibody, comprising a heavy chain variable region, wherein the heavy chain variable region comprises at least one complementarity determining region (CDR), wherein the CDR that is farthest from the N terminus is selected from SEQ ID NOs: 61, 67, 73, 79, 85, 89, 95, 101, 104, 112, 116, 121, 125, 128, 131, 136, 138, 142, 144, 147, 151, 154, and 158. In some embodiments, the CDR that is farthest from the N terminus is CDR3. The heavy chain CDR3 is conformationally located at the center of the antigen-binding site, and therefore is believed to make the most contact with antigen and provides the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa, S., Nature, 1983; 302 (5909), 575-581). The diversity in the heavy chain CDR3 is sufficient to produce most antibody specificities as well as desirable antigen binding affinity (Xu et al., Immunity, 2000; 13 (1), 37-45; Schier et al., Journal of molecular biology, 1996; 263 (4), 551-567).

In some embodiments, the heavy chain variable region comprises three CDRs, which are hCDR1, hCDR2, and hCDR3 from N terminus to C terminus, wherein the hCDR1 is selected from SEQ ID NOs: 59, 65, 71, 77, 83, 88, 93, 103, 110, 119, 135, 143, 146, 150, and 152; the hCDR2 is selected from SEQ ID NOs: 60, 66, 72, 78, 84, 94, 111, 118, 120, 124, 141, and 153; and the hCDR3 is selected from SEQ ID NOs: 61, 67, 73, 79, 85, 89, 95, 101, 104, 112, 116, 121, 125, 128, 131, 136, 138, 142, 144, 147, 151, 154, and 158.

In some embodiments, wherein the hCDR1, hCDR2, and hCDR3 comprise:
a. SEQ ID NO: 59, SEQ ID NO: 60, SEQ ID NO: 61, respectively;
b. SEQ ID NO: 65, SEQ ID NO: 66, SEQ ID NO: 67, respectively;
c. SEQ ID NO: 71, SEQ ID NO: 72, SEQ ID NO: 73, respectively;
d. SEQ ID NO: 77, SEQ ID NO: 78, SEQ ID NO: 79, respectively;
e. SEQ ID NO: 83, SEQ ID NO: 84, SEQ ID NO: 85, respectively;
f. SEQ ID NO: 88, SEQ ID NO: 84, SEQ ID NO: 89, respectively;
g. SEQ ID NO: 93, SEQ ID NO: 94, SEQ ID NO: 95, respectively;
h. SEQ ID NO: 59, SEQ ID NO: 78, SEQ ID NO: 79, respectively;
i. SEQ ID NO: 93, SEQ ID NO: 94, SEQ ID NO: 101, respectively;
j. SEQ ID NO: 103, SEQ ID NO: 94, SEQ ID NO: 104, respectively;
k. SEQ ID NO: 59, SEQ ID NO: 78, SEQ ID NO: 79, respectively;
l. SEQ ID NO: 110, SEQ ID NO: 111, SEQ ID NO: 112, respectively;
m. SEQ ID NO: 88, SEQ ID NO: 84, SEQ ID NO: 116, respectively;
n. SEQ ID NO: 93, SEQ ID NO: 94, SEQ ID NO: 101, respectively;
o. SEQ ID NO: 93, SEQ ID NO: 118, SEQ ID NO: 101, respectively;
p. SEQ ID NO: 119, SEQ ID NO: 120, SEQ ID NO: 121, respectively;
q. SEQ ID NO: 119, SEQ ID NO: 124, SEQ ID NO: 125, respectively;
r. SEQ ID NO: 77, SEQ ID NO: 78, SEQ ID NO: 128, respectively;
s. SEQ ID NO: 83, SEQ ID NO: 94, SEQ ID NO: 131, respectively;
t. SEQ ID NO: 135, SEQ ID NO: 84, SEQ ID NO: 136, respectively;
u. SEQ ID NO: 88, SEQ ID NO: 84, SEQ ID NO: 138, respectively;
v. SEQ ID NO: 83, SEQ ID NO: 141, SEQ ID NO: 142, respectively;
w. SEQ ID NO: 143, SEQ ID NO: 84, SEQ ID NO: 144, respectively;
x. SEQ ID NO: 146, SEQ ID NO: 94, SEQ ID NO: 147, respectively;
y. SEQ ID NO: 119, SEQ ID NO: 120, SEQ ID NO: 121, respectively;
z. SEQ ID NO: 119, SEQ ID NO: 124, SEQ ID NO: 125, respectively;
aa. SEQ ID NO: 150, SEQ ID NO: 84, SEQ ID NO: 151, respectively;
bb. SEQ ID NO: 152, SEQ ID NO: 153, SEQ ID NO: 154, respectively; or
cc. SEQ ID NO: 88, SEQ ID NO: 94, SEQ ID NO: 158, respectively.

In some embodiments, the heavy chain variable region comprises four framework regions (FRs), which are hFR1, hFR2, hFR3, and hFR4 from N terminus to C terminus, wherein the hFR1 is selected from SEQ ID NOs: 160, 168, 175, 188, 194, 204, 210, 224, 233, 284, 259, 265, 268, 271, 274, 281, 295, and 305; the hFR2 is selected from SEQ ID NOs: 161, 169, 176, 183, 205, 221, 225, 237, 245, 251, 261, 266, 275, 282, and 296; the hFR3 is selected from SEQ ID NOs: 162, 170, 177, 184, 189, 195, 200, 206, 211, 216, 226, 231, 234, 239, 242, 246, 252, 254, 260, 262, 267, 272, 276, 297, and 306; and the hFR4 is selected from SEQ ID NOs: 163, 178, 190, 196, 212, 217, 227, 255, 269, and 298.

In some embodiments, the hFR1, hFR2, hFR3, and hFR4 comprise:
a. SEQ ID NO: 160, SEQ ID NO: 161, SEQ ID NO: 162, SEQ ID NO: 163, respectively;
b. SEQ ID NO: 168, SEQ ID NO: 169, SEQ ID NO: 170, SEQ ID NO: 163, respectively;
c. SEQ ID NO: 175, SEQ ID NO: 176, SEQ ID NO: 177, SEQ ID NO: 178, respectively;
d. SEQ ID NO: 160, SEQ ID NO: 183, SEQ ID NO: 184, SEQ ID NO: 178, respectively;
e. SEQ ID NO: 188, SEQ ID NO: 169, SEQ ID NO: 189, SEQ ID NO: 190, respectively;
f. SEQ ID NO: 194, SEQ ID NO: 169, SEQ ID NO: 195, SEQ ID NO: 196, respectively;
g. SEQ ID NO: 194, SEQ ID NO: 169, SEQ ID NO: 200, SEQ ID NO: 163, respectively;
h. SEQ ID NO: 204, SEQ ID NO: 205, SEQ ID NO: 206, SEQ ID NO: 178, respectively;
i. SEQ ID NO: 210, SEQ ID NO: 169, SEQ ID NO: 211, SEQ ID NO: 212, respectively;
j. SEQ ID NO: 194, SEQ ID NO: 169, SEQ ID NO: 216, SEQ ID NO: 217, respectively;
k. SEQ ID NO: 204, SEQ ID NO: 221, SEQ ID NO: 206, SEQ ID NO: 178, respectively;
l. SEQ ID NO: 224, SEQ ID NO: 225, SEQ ID NO: 226, SEQ ID NO: 227, respectively;
m. SEQ ID NO: 194, SEQ ID NO: 169, SEQ ID NO: 231, SEQ ID NO: 163, respectively;
n. SEQ ID NO: 233, SEQ ID NO: 169, SEQ ID NO: 234, SEQ ID NO: 178, respectively;
o. SEQ ID NO: 210, SEQ ID NO: 237, SEQ ID NO: 211, SEQ ID NO: 212, respectively;
p. SEQ ID NO: 194, SEQ ID NO: 169, SEQ ID NO: 239, SEQ ID NO: 190, respectively;
q. SEQ ID NO: 194, SEQ ID NO: 169, SEQ ID NO: 242, SEQ ID NO: 178, respectively;
r. SEQ ID NO: 284, SEQ ID NO: 183, SEQ ID NO: 184, SEQ ID NO: 163, respectively;
s. SEQ ID NO: 194, SEQ ID NO: 245, SEQ ID NO: 246, SEQ ID NO: 163, respectively;
t. SEQ ID NO: 188, SEQ ID NO: 251, SEQ ID NO: 252, SEQ ID NO: 163, respectively;
u. SEQ ID NO: 188, SEQ ID NO: 169, SEQ ID NO: 254, SEQ ID NO: 255, respectively;
v. SEQ ID NO: 259, SEQ ID NO: 169, SEQ ID NO: 260, SEQ ID NO: 190, respectively;
w. SEQ ID NO: 194, SEQ ID NO: 261, SEQ ID NO: 262, SEQ ID NO: 163, respectively;
x. SEQ ID NO: 265, SEQ ID NO: 266, SEQ ID NO: 267, SEQ ID NO: 163, respectively;
y. SEQ ID NO: 268, SEQ ID NO: 169, SEQ ID NO: 239, SEQ ID NO: 269, respectively;
z. SEQ ID NO: 194, SEQ ID NO: 169, SEQ ID NO: 200, SEQ ID NO: 190, respectively;
aa. SEQ ID NO: 271, SEQ ID NO: 169, SEQ ID NO: 272, SEQ ID NO: 190, respectively;
bb. SEQ ID NO: 274, SEQ ID NO: 275, SEQ ID NO: 276, SEQ ID NO: 178, respectively; or
cc. SEQ ID NO: 281, SEQ ID NO: 282, SEQ ID NO: 254, SEQ ID NO: 163, respectively.
dd. SEQ ID NO: 295, SEQ ID NO: 296, SEQ ID NO: 297, SEQ ID NO: 298, respectively;
ee. SEQ ID NO: 305, SEQ ID NO: 296, SEQ ID NO: 306, SEQ ID NO: 298, respectively;
ff. SEQ ID NO: 295, SEQ ID NO: 296, SEQ ID NO: 306, SEQ ID NO: 298, respectively;
gg. SEQ ID NO: 305, SEQ ID NO: 296, SEQ ID NO: 297, SEQ ID NO: 298, respectively;

In some embodiments, the CDRs and FRs in the heavy chain variable region are operably connected in the order of hFR1-hCDR1-hFR2-hCDR2-hFR3-hCDR3-hFR4 from N terminus to C terminus.

In some embodiments, the antibody further comprises a heavy chain constant region, wherein the heavy chain constant region comprises three domains, CH1, CH2, and CH3, from N-terminus to C terminus, and wherein the CH1, CH2, and CH3 are
SEQ ID NO. 307, SEQ ID NO. 309, and SEQ ID NO. 310, respectively; or
SEQ ID NO: 307, SEQ ID NO. 312, and SEQ ID NO. 313, respectively.

In some embodiments, the heavy chain variable region comprises an amino acid sequence with at least 80%, 85%, 90%, 95%, 96%, 97%, 98%, 99%, or 100% identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 285, 287, 290, and 291.

In some embodiments, the heavy chain variable region comprises an amino acid sequence of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 285, 287, 290, and 291.

In some embodiments, the antibody described herein further comprises a light chain variable region, wherein the light chain variable region comprises at least one CDR, wherein the CDR that is farthest from the N terminus is selected from SEQ ID NOs: 64, 70, 76, 82, 87, 92, 98, 100, 102, 107, 109, 115, 117, 123, 127, 130, 134, 137, 140, 145, 157, and 159.

In some embodiments, the light chain variable region comprises three CDRs, which are 1CDR1, 1CDR2, and 1CDR3 from N terminus to C terminus, wherein the 1CDR1 is selected from SEQ ID NOs: 62, 68, 74, 80, 86, 90, 96, 99, 105, 108, 113, 122, 126, 129, 132, 148, 149, and 155; the 1CDR2 is selected from SEQ ID NOs: 63, 69, 75, 81, 91, 97, 106, 114, 133, 139, and 156; and the ICDR3 is selected from SEQ ID NOs: 64, 70, 76, 82, 87, 92, 98, 100, 102, 107, 109, 115, 117, 123, 127, 130, 134, 137, 140, 145, 157, and 159.

In some embodiments, the ICDR1, ICDR2, and ICDR3 comprise:
a. SEQ ID NO: 62, SEQ ID NO: 63, SEQ ID NO: 64, respectively;
b. SEQ ID NO: 68, SEQ ID NO: 69, SEQ ID NO: 70, respectively;
c. SEQ ID NO: 74, SEQ ID NO: 75, SEQ ID NO: 76, respectively;
d. SEQ ID NO: 80, SEQ ID NO: 81, SEQ ID NO: 82, respectively;
e. SEQ ID NO: 86, SEQ ID NO: 69, SEQ ID NO: 87, respectively;
f. SEQ ID NO: 90, SEQ ID NO: 91, SEQ ID NO: 92, respectively;
g. SEQ ID NO: 96, SEQ ID NO: 97, SEQ ID NO: 98, respectively;
h. SEQ ID NO: 99, SEQ ID NO: 69, SEQ ID NO: 100, respectively;
i. SEQ ID NO: 96, SEQ ID NO: 97, SEQ ID NO: 102, respectively;
j. SEQ ID NO: 105, SEQ ID NO: 106, SEQ ID NO: 107, respectively;
l. SEQ ID NO: 113, SEQ ID NO: 114, SEQ ID NO: 115, respectively;
m. SEQ ID NO: 105, SEQ ID NO: 106, SEQ ID NO: 117, respectively;
n. SEQ ID NO: 96, SEQ ID NO: 97, SEQ ID NO: 102, respectively;
o. SEQ ID NO: 96, SEQ ID NO: 97, SEQ ID NO: 102, respectively;
p. SEQ ID NO: 122, SEQ ID NO: 69, SEQ ID NO: 123, respectively;
q. SEQ ID NO: 126, SEQ ID NO: 69, SEQ ID NO: 127, respectively;
r. SEQ ID NO: 129, SEQ ID NO: 69, SEQ ID NO: 130, respectively;
s. SEQ ID NO: 132, SEQ ID NO: 133, SEQ ID NO: 134, respectively;
t. SEQ ID NO: 126, SEQ ID NO: 69, SEQ ID NO: 137, respectively; t.
u. SEQ ID NO: 132, SEQ ID NO: 139, SEQ ID NO: 140, respectively;
v. SEQ ID NO: 122, SEQ ID NO: 69, SEQ ID NO: 123, respectively;
w. SEQ ID NO: 90, SEQ ID NO: 91, SEQ ID NO: 145, respectively;
x. SEQ ID NO: 148, SEQ ID NO: 69, SEQ ID NO: 109, respectively;
y. SEQ ID NO: 149, SEQ ID NO: 63, SEQ ID NO: 109, respectively;
z. SEQ ID NO: 122, SEQ ID NO: 69, SEQ ID NO: 123, respectively;
aa. SEQ ID NO: 126, SEQ ID NO: 69, SEQ ID NO: 130, respectively;
bb. SEQ ID NO: 155, SEQ ID NO: 156, SEQ ID NO: 157, respectively; or
cc. SEQ ID NO: 155, SEQ ID NO: 156, SEQ ID NO: 159, respectively.

In some embodiments, the light chain variable region comprises four framework regions, which are IFR1, IFR2, IFR3, and IFR4 from N terminus to C terminus, wherein the IFR1 is selected from SEQ ID NOs: 164, 171, 179, 185, 191, 197, 201, 207, 213, 218, 222, 228, 232, 247, 256, 263, 270, 277, 283, 299, and 303; the 1FR2 is selected from SEQ ID NOs: 165, 172, 180, 186, 192, 198, 202, 208, 214, 219, 223, 229, 235, 240, 243, 248, 257, 264, 278, 300, and 304; the IFR3 is selected from SEQ ID NOs: 166, 173, 181, 187, 193, 199, 203, 209, 220, 230, 236, 238, 241, 244, 249, 253, 258, 273, 279, and 301; and the 1FR4 is selected from SEQ ID NOs: 167, 174, 182, 215, 250, 280, and 302.

In some embodiments, the IFR1, 1FR2, 1FR3, and IFR4 comprise:
a. SEQ ID NO: 164, SEQ ID NO: 165, SEQ ID NO: 166, SEQ ID NO: 167, respectively;
b. SEQ ID NO: 171, SEQ ID NO: 172, SEQ ID NO: 173, SEQ ID NO: 174, respectively;
c. SEQ ID NO: 179, SEQ ID NO: 180, SEQ ID NO: 181, SEQ ID NO: 182, respectively;
d. SEQ ID NO: 185, SEQ ID NO: 186, SEQ ID NO: 187, SEQ ID NO: 174, respectively;
e. SEQ ID NO: 191, SEQ ID NO: 192, SEQ ID NO: 193, SEQ ID NO: 174, respectively;
f. SEQ ID NO: 197, SEQ ID NO: 198, SEQ ID NO: 199, SEQ ID NO: 174, respectively;
g. SEQ ID NO: 201, SEQ ID NO: 202, SEQ ID NO: 203, SEQ ID NO: 167, respectively;
h. SEQ ID NO: 207, SEQ ID NO: 208, SEQ ID NO: 209, SEQ ID NO: 174, respectively;
i. SEQ ID NO: 213, SEQ ID NO: 214, SEQ ID NO: 203, SEQ ID NO: 215, respectively;
j. SEQ ID NO: 218, SEQ ID NO: 219, SEQ ID NO: 220, SEQ ID NO: 174, respectively;
k. SEQ ID NO: 222, SEQ ID NO: 223, SEQ ID NO: 209, SEQ ID NO: 174, respectively;
l. SEQ ID NO: 228, SEQ ID NO: 229, SEQ ID NO: 230, SEQ ID NO: 174, respectively;
m. SEQ ID NO: 232, SEQ ID NO: 219, SEQ ID NO: 220, SEQ ID NO: 167, respectively;
n. SEQ ID NO: 201, SEQ ID NO: 235, SEQ ID NO: 236, SEQ ID NO: 174, respectively;
o. SEQ ID NO: 201, SEQ ID NO: 214, SEQ ID NO: 238, SEQ ID NO: 215, respectively;
p. SEQ ID NO: 171, SEQ ID NO: 240, SEQ ID NO: 241, SEQ ID NO: 174, respectively;
q. SEQ ID NO: 171, SEQ ID NO: 243, SEQ ID NO: 244, SEQ ID NO: 174, respectively;
r. SEQ ID NO: 222, SEQ ID NO: 165, SEQ ID NO: 209, SEQ ID NO: 174, respectively;
s. SEQ ID NO: 247, SEQ ID NO: 248, SEQ ID NO: 249, SEQ ID NO: 250, respectively;
t. SEQ ID NO: 171, SEQ ID NO: 243, SEQ ID NO: 253, SEQ ID NO: 167, respectively;
u. SEQ ID NO: 256, SEQ ID NO: 257, SEQ ID NO: 258, SEQ ID NO: 174, respectively;
v. SEQ ID NO: 171, SEQ ID NO: 240, SEQ ID NO: 241, SEQ ID NO: 174, respectively;
w. SEQ ID NO: 263, SEQ ID NO: 264, SEQ ID NO: 199, SEQ ID NO: 174, respectively;
x. SEQ ID NO: 222, SEQ ID NO: 165, SEQ ID NO: 209, SEQ ID NO: 174, respectively;
y. SEQ ID NO: 270, SEQ ID NO: 165, SEQ ID NO: 166, SEQ ID NO: 174, respectively;
z. SEQ ID NO: 171, SEQ ID NO: 240, SEQ ID NO: 241, SEQ ID NO: 174, respectively;
aa. SEQ ID NO: 171, SEQ ID NO: 243, SEQ ID NO: 273, SEQ ID NO: 174, respectively;
bb. SEQ ID NO: 277, SEQ ID NO: 278, SEQ ID NO: 279, SEQ ID NO: 280, respectively; or
cc. SEQ ID NO: 283, SEQ ID NO: 278, SEQ ID NO: 279, SEQ ID NO: 174, respectively.
dd. SEQ ID NO: 299, SEQ ID NO: 300, SEQ ID NO: 301, SEQ ID NO: 302, respectively;
ee. SEQ ID NO: 303, SEQ ID NO: 304, SEQ ID NO: 301, SEQ ID NO: 302, respectively;
ff. SEQ ID NO: 299, SEQ ID NO: 304, SEQ ID NO: 301, SEQ ID NO: 302, respectively.

In some embodiments, the CDRs and FRs in the light chain variable region are operably connected in the order of IFR1-1CDR1-1FR2-1CDR2-1FR3-1CDR3-IFR4 from N terminus to C terminus.

In some embodiments, the antibody further comprises a light chain constant region (CL), wherein the CL is SEQ ID NO: 311.

In some embodiments, the light chain variable region comprises an amino acid sequence with at least 80%, 85%, 90%, 95%, 96%, 97%, 98%, 99%, or 100% identity to any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

In some embodiments, the light chain variable region comprises an amino acid sequence of any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

In some embodiments, the heavy chain variable region and the light chain variable region comprise an amino acid sequence with at least 80%, 85%, 90%, 95%, 96%, 97%, 98%, 99%, or 100% identity to:
a. SEQ ID NO: 1, and SEQ ID NO: 2, respectively;
b. SEQ ID NO: 3, and SEQ ID NO: 4, respectively;
c. SEQ ID NO: 5, and SEQ ID NO: 6, respectively;
d. SEQ ID NO: 7, and SEQ ID NO: 8, respectively;
e. SEQ ID NO: 9, and SEQ ID NO: 10, respectively;
f. SEQ ID NO: 11, and SEQ ID NO: 12, respectively;
g. SEQ ID NO: 13, and SEQ ID NO: 14, respectively;
h. SEQ ID NO: 15, and SEQ ID NO: 16, respectively;
i. SEQ ID NO: 17, and SEQ ID NO: 18, respectively;
j. SEQ ID NO: 19, and SEQ ID NO: 20, respectively;
k. SEQ ID NO: 21, and SEQ ID NO: 22, respectively;
l. SEQ ID NO: 23, and SEQ ID NO: 24, respectively;
m. SEQ ID NO: 25, and SEQ ID NO: 26, respectively;
n. SEQ ID NO: 27, and SEQ ID NO: 28, respectively;
o. SEQ ID NO: 29, and SEQ ID NO: 30, respectively;
p. SEQ ID NO: 31, and SEQ ID NO: 32, respectively;
q. SEQ ID NO: 33, and SEQ ID NO: 34, respectively;
r. SEQ ID NO: 35, and SEQ ID NO: 36, respectively;
s. SEQ ID NO: 37, and SEQ ID NO: 38, respectively;
t. SEQ ID NO: 39, and SEQ ID NO: 40, respectively;
u. SEQ ID NO: 41, and SEQ ID NO: 42, respectively;
v. SEQ ID NO: 43, and SEQ ID NO: 32, respectively;
w. SEQ ID NO: 45, and SEQ ID NO: 46, respectively;
x. SEQ ID NO: 47, and SEQ ID NO: 48, respectively;
y. SEQ ID NO: 49, and SEQ ID NO: 50, respectively;
z. SEQ ID NO: 51, and SEQ ID NO: 32, respectively;
aa. SEQ ID NO: 53, and SEQ ID NO: 54, respectively;
bb. SEQ ID NO: 55, and SEQ ID NO: 56, respectively; or
cc. SEQ ID NO: 57, and SEQ ID NO: 58, respectively.
dd. SEQ ID NO: 285, and SEQ ID NO: 286, respectively;
ee. SEQ ID NO: 287, and SEQ ID NO: 288, respectively;
ff. SEQ ID NO: 287, and SEQ ID NO: 289, respectively;
gg. SEQ ID NO: 290, and SEQ ID NO: 288, respectively;
hh. SEQ ID NO: 290, and SEQ ID NO: 286, respectively;
ii. SEQ ID NO: 291, and SEQ ID NO: 286, respectively;
jj. SEQ ID NO: 292, and SEQ ID NO: 293, respectively;
kk. SEQ ID NO: 294, and SEQ ID NO: 293, respectively.

In some embodiments, the heavy chain variable region and the light chain variable region comprise:
a. SEQ ID NO: 1, and SEQ ID NO: 2, respectively;
b. SEQ ID NO: 3, and SEQ ID NO: 4, respectively;
c. SEQ ID NO: 5, and SEQ ID NO: 6, respectively;
d. SEQ ID NO: 7, and SEQ ID NO: 8, respectively;
e. SEQ ID NO: 9, and SEQ ID NO: 10, respectively;
f. SEQ ID NO: 11, and SEQ ID NO: 12, respectively;
g. SEQ ID NO: 13, and SEQ ID NO: 14, respectively;
h. SEQ ID NO: 15, and SEQ ID NO: 16, respectively;
i. SEQ ID NO: 17, and SEQ ID NO: 18, respectively;
j. SEQ ID NO: 19, and SEQ ID NO: 20, respectively;
k. SEQ ID NO: 21, and SEQ ID NO: 22, respectively;
l. SEQ ID NO: 23, and SEQ ID NO: 24, respectively;
m. SEQ ID NO: 25, and SEQ ID NO: 26, respectively;
n. SEQ ID NO: 27, and SEQ ID NO: 28, respectively;
o. SEQ ID NO: 29, and SEQ ID NO: 30, respectively;
p. SEQ ID NO: 31, and SEQ ID NO: 32, respectively;
q. SEQ ID NO: 33, and SEQ ID NO: 34, respectively;
r. SEQ ID NO: 35, and SEQ ID NO: 36, respectively;
S. SEQ ID NO: 37, and SEQ ID NO: 38, respectively;
t. SEQ ID NO: 39, and SEQ ID NO: 40, respectively;
u. SEQ ID NO: 41, and SEQ ID NO: 42, respectively;
v. SEQ ID NO: 43, and SEQ ID NO: 32, respectively;
w. SEQ ID NO: 45, and SEQ ID NO: 46, respectively;
x. SEQ ID NO: 47, and SEQ ID NO: 48, respectively;
y. SEQ ID NO: 49, and SEQ ID NO: 50, respectively;
z. SEQ ID NO: 51, and SEQ ID NO: 32, respectively;
aa. SEQ ID NO: 53, and SEQ ID NO: 54, respectively;
bb. SEQ ID NO: 55, and SEQ ID NO: 56, respectively; or
cc. SEQ ID NO: 57, and SEQ ID NO: 58, respectively,
dd. SEQ ID NO: 285, and SEQ ID NO: 286, respectively;
ee. SEQ ID NO: 287, and SEQ ID NO: 288, respectively;
ff. SEQ ID NO: 287, and SEQ ID NO: 289, respectively;
gg. SEQ ID NO: 290, and SEQ ID NO: 288, respectively;
hh. SEQ ID NO: 290, and SEQ ID NO: 286, respectively;
ii. SEQ ID NO: 291, and SEQ ID NO: 286, respectively;
jj. SEQ ID NO: 292, and SEQ ID NO: 293, respectively;
kk. SEQ ID NO: 294, and SEQ ID NO: 293, respectively.

In another aspect, the present disclosure provides an antibody, comprising a light chain variable region, wherein the light chain variable region comprises at least one CDR, wherein the CDR that is farthest from the N terminus is selected from SEQ ID NOs: 64, 70, 76, 82, 87, 92, 98, 100, 102, 107, 109, 115, 117, 123, 127, 130, 134, 137, 140, 145, 157, and 159.

In some embodiments, the light chain variable region comprises three CDRs, which are ICDR1, ICDR2, and ICDR3 from N terminus to C terminus, wherein the ICDR1 is selected from SEQ ID NOs: 62, 68, 74, 80, 86, 90, 96, 99, 105, 108, 113, 122, 126, 129, 132, 148, 149, and 155; the ICDR2 is selected from SEQ ID NOs: 63, 69, 75, 81, 91, 97, 106, 114, 133, 139, and 156; and the ICDR3 is selected from SEQ ID NOs: 64, 70, 76, 82, 87, 92, 98, 100, 102, 107, 109, 115, 117, 123, 127, 130, 134, 137, 140, 145, 157, and 159.

In some embodiments, the 1CDR1, 1CDR2, and ICDR3 comprise:
a. SEQ ID NO: 62, SEQ ID NO: 63, SEQ ID NO: 64, respectively;
b. SEQ ID NO: 68, SEQ ID NO: 69, SEQ ID NO: 70, respectively;
c. SEQ ID NO: 74, SEQ ID NO: 75, SEQ ID NO: 76, respectively;
d. SEQ ID NO: 80, SEQ ID NO: 81, SEQ ID NO: 82, respectively;
e. SEQ ID NO: 86, SEQ ID NO: 69, SEQ ID NO: 87, respectively;
f. SEQ ID NO: 90, SEQ ID NO: 91, SEQ ID NO: 92, respectively;
g. SEQ ID NO: 96, SEQ ID NO: 97, SEQ ID NO: 98, respectively;
h. SEQ ID NO: 99, SEQ ID NO: 69, SEQ ID NO: 100, respectively;
i. SEQ ID NO: 96, SEQ ID NO: 97, SEQ ID NO: 102, respectively;
j. SEQ ID NO: 105, SEQ ID NO: 106, SEQ ID NO: 107, respectively;
k. SEQ ID NO: 108, SEQ ID NO: 69, SEQ ID NO: 109, respectively;
l. SEQ ID NO: 113, SEQ ID NO: 114, SEQ ID NO: 115, respectively;
m. SEQ ID NO: 105, SEQ ID NO: 106, SEQ ID NO: 117, respectively;
n. SEQ ID NO: 96, SEQ ID NO: 97, SEQ ID NO: 102, respectively;
o. SEQ ID NO: 96, SEQ ID NO: 97, SEQ ID NO: 102, respectively;
p. SEQ ID NO: 122, SEQ ID NO: 69, SEQ ID NO: 123, respectively;
q. SEQ ID NO: 126, SEQ ID NO: 69, SEQ ID NO: 127, respectively;
r. SEQ ID NO: 129, SEQ ID NO: 69, SEQ ID NO: 130, respectively;
s. SEQ ID NO: 132, SEQ ID NO: 133, SEQ ID NO: 134, respectively;
t. SEQ ID NO: 126, SEQ ID NO: 69, SEQ ID NO: 137, respectively;
u. SEQ ID NO: 132, SEQ ID NO: 139, SEQ ID NO: 140, respectively;
v. SEQ ID NO: 122, SEQ ID NO: 69, SEQ ID NO: 123, respectively;
w. SEQ ID NO: 90, SEQ ID NO: 91, SEQ ID NO: 145, respectively;
x. SEQ ID NO: 148, SEQ ID NO: 69, SEQ ID NO: 109, respectively;
y. SEQ ID NO: 149, SEQ ID NO: 63, SEQ ID NO: 109, respectively;
z. SEQ ID NO: 122, SEQ ID NO: 69, SEQ ID NO: 123, respectively;
aa. SEQ ID NO: 126, SEQ ID NO: 69, SEQ ID NO: 130, respectively;
bb. SEQ ID NO: 155, SEQ ID NO: 156, SEQ ID NO: 157, respectively; or
cc. SEQ ID NO: 155, SEQ ID NO: 156, SEQ ID NO: 159, respectively.

In some embodiments, the light chain variable region comprises four framework regions, which are IFR1, IFR2, IFR3, and IFR4 from N terminus to C terminus, wherein the IFR1 is selected from SEQ ID NOs: 164, 171, 179, 185, 191, 197, 201, 207, 213, 218, 222, 228, 232, 247, 256, 263, 270, 277, 283, 299, and 303; the IFR2 is selected from SEQ ID NOs: 165, 172, 180, 186, 192, 198, 202, 208, 214, 219, 223, 229, 235, 240, 243, 248, 257, 264, 278, 300, and 304; the 1FR3 is selected from SEQ ID NOs: 166, 173, 181, 187, 193, 199, 203, 209, 220, 230, 236, 238, 241, 244, 249, 253, 258, 273, 279, and 301; and the IFR4 is selected from SEQ ID NOs: 167, 174, 182, 215, 250, 280, and 302.

In some embodiments, the IFR1, 1FR2, IFR3, and IFR4 comprise:
a. SEQ ID NO: 164, SEQ ID NO: 165, SEQ ID NO: 166, SEQ ID NO: 167, respectively;
b. SEQ ID NO: 171, SEQ ID NO: 172, SEQ ID NO: 173, SEQ ID NO: 174, respectively;
c. SEQ ID NO: 179, SEQ ID NO: 180, SEQ ID NO: 181, SEQ ID NO: 182, respectively;
d. SEQ ID NO: 185, SEQ ID NO: 186, SEQ ID NO: 187, SEQ ID NO: 174, respectively;
e. SEQ ID NO: 191, SEQ ID NO: 192, SEQ ID NO: 193, SEQ ID NO: 174, f. SEQ ID NO: 197, SEQ ID NO: 198, SEQ ID NO: 199, SEQ ID NO: 174,
g. SEQ ID NO: 201, SEQ ID NO: 202, SEQ ID NO: 203, SEQ ID NO: 167, respectively;
h. SEQ ID NO: 207, SEQ ID NO: 208, SEQ ID NO: 209, SEQ ID NO: 174, respectively;
i. SEQ ID NO: 213, SEQ ID NO: 214, SEQ ID NO: 203, SEQ ID NO: 215, respectively;
j. SEQ ID NO: 218, SEQ ID NO: 219, SEQ ID NO: 220, SEQ ID NO: 174, respectively;
k. SEQ ID NO: 222, SEQ ID NO: 223, SEQ ID NO: 209, SEQ ID NO: 174, respectively;
l. SEQ ID NO: 228, SEQ ID NO: 229, SEQ ID NO: 230, SEQ ID NO: 174, respectively;
m. SEQ ID NO: 232, SEQ ID NO: 219, SEQ ID NO: 220, SEQ ID NO: 167, respectively;
n. SEQ ID NO: 201, SEQ ID NO: 235, SEQ ID NO: 236, SEQ ID NO: 174, respectively;
o. SEQ ID NO: 201, SEQ ID NO: 214, SEQ ID NO: 238, SEQ ID NO: 215, respectively;
p. SEQ ID NO: 171, SEQ ID NO: 240, SEQ ID NO: 241, SEQ ID NO: 174, respectively;
q. SEQ ID NO: 171, SEQ ID NO: 243, SEQ ID NO: 244, SEQ ID NO: 174, respectively;
r. SEQ ID NO: 222, SEQ ID NO: 165, SEQ ID NO: 209, SEQ ID NO: 174, respectively;
s. SEQ ID NO: 247, SEQ ID NO: 248, SEQ ID NO: 249, SEQ ID NO: 250, respectively;
t. SEQ ID NO: 171, SEQ ID NO: 243, SEQ ID NO: 253, SEQ ID NO: 167, respectively;
u. SEQ ID NO: 256, SEQ ID NO: 257, SEQ ID NO: 258, SEQ ID NO: 174, respectively;
v. SEQ ID NO: 171, SEQ ID NO: 240, SEQ ID NO: 241, SEQ ID NO: 174, respectively;
w. SEQ ID NO: 263, SEQ ID NO: 264, SEQ ID NO: 199, SEQ ID NO: 174, respectively;
x. SEQ ID NO: 222, SEQ ID NO: 165, SEQ ID NO: 209, SEQ ID NO: 174, respectively;
y. SEQ ID NO: 270, SEQ ID NO: 165, SEQ ID NO: 166, SEQ ID NO: 174, respectively;
z. SEQ ID NO: 171, SEQ ID NO: 240, SEQ ID NO: 241, SEQ ID NO: 174, respectively;
aa. SEQ ID NO: 171, SEQ ID NO: 243, SEQ ID NO: 273, SEQ ID NO: 174, respectively;
bb. SEQ ID NO: 277, SEQ ID NO: 278, SEQ ID NO: 279, SEQ ID NO: 280, respectively; or
cc. SEQ ID NO: 283, SEQ ID NO: 278, SEQ ID NO: 279, SEQ ID NO: 174, respectively.
dd. SEQ ID NO: 299, SEQ ID NO: 300, SEQ ID NO: 301, SEQ ID NO: 302, respectively;
ee. SEQ ID NO: 303, SEQ ID NO: 304, SEQ ID NO: 301, SEQ ID NO: 302, respectively;
ff. SEQ ID NO: 299, SEQ ID NO: 304, SEQ ID NO: 301, SEQ ID NO: 302, respectively;

In some embodiments, the CDRs and FRs in the light chain variable region are operably connected in the order of IFR1-ICDR1-IFR2-1CDR2-IFR3-ICDR3-IFR4 from N terminus to C terminus.

In some embodiments, the antibody further comprises a light chain constant region (CL), wherein the CL is SEQ ID NO: 311.

In some embodiments, the light chain variable region comprises an amino acid sequence with at least 80%, 85%, 90%, 95%, 96%, 97%, 98%, 99%, or 100% identity to any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

In some embodiments, the light chain variable region comprises an amino acid sequence of any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

In some embodiments, the antibody described herein is a chimeric antibody. In some embodiments, the chimeric antibody is derived from mouse antibody and/or humanized antibody. As used herein, “chimeric antibody” refers to an antibody having a portion of heavy and/or light chain derived from one species, and the rest of the heavy and/or light chain derived from a different species. In some embodiments, a chimeric antibody comprises a constant region derived from human and a variable region from a non-human animal such as mouse. In some embodiments, the non-human animal is a mammal, for example, a mouse, a rat, or a rabbit. Chimeric antibody can also include an antibody where the variable domain and constant domain are each derived from two different sources even if both are from the same species

In some embodiments, the antibody described herein is a humanized or partially humanized antibody. As used herein, “humanized antibody” refers to antibodies that are modified to reduce their immunogenicity in humans. Humanization can be carried out in any method known in the art. In some embodiments, the humanization is carried out by grafting non-human origin CDR sequences onto human framework sequences. In some embodiments, additional framework region modifications may be made within the human framework sequences. In some embodiments, the humanization is carried out by augmented binary substitution (Townsend et al., Proceedings of the National Academy of Sciences 112.50 (2015): 15354-15359). As used herein, “partially-humanized” or “partial humanization” means that a portion of the antibody is modified to reduce its immunogenicity in humans. For example, in some embodiments, one pair of heavy chain and light chain is modified to reduce immunogenicity in humans.

In some embodiments, the antibody described herein is a monoclonal antibody. As used herein, “monoclonal antibodies (mAb)” refers to a preparation of antibody molecules of single molecular composition. A monoclonal antibody composition displays a single binding specificity and affinity for a particular epitope. In some embodiments, a monoclonal antibody exists in a homogeneous or substantially homogeneous population.

In some embodiments, the antibody described herein is a polyclonal antibody. As used herein, “polyclonal antibodies” refers to a preparation of antibody molecules of different molecular composition, which recognize a variety of epitopes on an antigen.

In some embodiments, the antibody described herein is a multispecific antibody. In some embodiments, the antibody described herein is a bispecific antibody. As used herein, “multispecific antibody” refers to an antibody which is capable of specifically binding to more than one epitope. “Bispecific antibody” refers to an antibody which is capable of specifically binding to two unique epitopes.

In some embodiments, the antibody described herein is a recombinant antibody. As used herein, “recombinant antibody” includes all antibodies that are prepared, expressed, created or isolated by recombinant means, such as, but are not limited to antibodies isolated from a host cell transformed to express the antibody, antibodies isolated from a recombinant, combinatorial antibody library, and antibodies prepared, expressed, created or isolated by any other means that involve splicing of immunoglobulin gene sequences to other DNA sequences.

In some embodiments, the antibody described herein is a heavy-chain antibody. As used herein, “heavy-chain antibody” refers to an antibody consisting of two heavy chains, without light chains.

In some embodiments, the antibody described herein is a single domain antibody. As used herein, “single domain antibody (sdAb)” or “nanobody”, which are used interchangeably, refers to an antibody fragment consisting of a single monomeric variable antibody domain.

In some embodiments, the antibody described herein is operably linked to a cytotoxic agent. In some embodiments, the antibody is linked to a cytotoxic agent by a short peptide. In some embodiments, the antibody is directly fused with a cytotoxic agent by recombinant means. As used herein, “cytotoxic agent,” “cytotoxic substance,” or “cytotoxic drug” refers to a substance that is toxic to cells, and/or is capable of killing cells, including cancer cells. Some exemplary cytotoxic agents are Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vemurafenib, Vinblastine, Vincristine, and Vindesine.

In some embodiments, the antibody described herein is an ADCC-enhanced antibody. In some embodiments, the antibody is an afucosylated antibody. In some embodiments, the antibody comprises a Fc region that is engineered to enhance ADCC.

“Antibody-dependent cell-mediated cytotoxicity” and “ADCC” refer to a cell-mediated reaction in which effector cells that express Fc receptors (FcRs) recognize bound antibody or antigen-binding fragment on a target cell and subsequently cause lysis of the target cell. “ADCC activity” refers to the ability of the antibody or antigen-binding fragment which is bound on the target cell to elicit an ADCC reaction.

ADCC activity is measured with in vivo assay as described in Example 9. ADCC activity was determined by calculating EC50 of antibody response and maximum response efficacy. In some embodiments, ADCC activity of an antibody is enhanced by making the antibody fucose-free (afucoyslated). In some embodiments, ADCC activity of an antibody is enhanced by engineered its Fc region to enhance its affinity to the Fc receptor on the target cell. Example 7 further discloses the details of these two methods of enhancing ADCC activity of an antibody.

TABLE 2

| | | |
|---|---|---|
| mAb1-VH | SEQ ID NO: 1 | QAYLQQSGAELVRPGASVKMSCKASG |
| | | YTFTSYNMHWVKQTPRQGLEWIGSIY |
| | | PENGDTSYNQKFRDKATLTVDKSSSTA |
| | | YMQLSSLTSEDSAVYFCARPFDYWGQ |
| | | GTTLTVSS |
| mAb1-VL | SEQ ID NO: 2 | SIVMTQTPKFLPISAGDRVTMTCKASQ |
| | | SVGNNVAWYQQKPGQSPKLLIYYASN |
| | | RYTGVPDRFTGSGSGTDFTFTISSVQVE |
| | | DLAVYFCQQFYSSPYTFGSGTKLEIK |
| mAb2-VH | SEQ ID NO: 3 | EVQLQQSGTVLARPGASVKMSCRASG |
| | | YTFTDYWMHWVKQRPGQGLEWIGAI |
| | | YPGNTDTDYNQKFKGKAKLTAVTSAS |
| | | SAYMELSSLTNEDSAVYYCTGFNLDY |
| | | WGQGTTLTVSS |
| mAb2-VL | SEQ ID NO: 4 | DIVMTQSQKFMSTSVGDRVSVTCKAS |
| | | QNVGTSVAWYQKKPGQSPKVVIYSAS |
| | | TRYTGVPDRFTGSGLGTDFTLTISNVQS |
| | | EDLAEYFCQQYGSSPYTFGGGTKLEIK |
| mAb3-VH | SEQ ID NO: 5 | DVQLQESGPGLVKPSQSLSLTCSVTGD |
| | | SITSGYYWNWIRQFPGNKLEWMGYIS |
| | | YEGSNNYNPSLKNRISITRDTSKNQFFL |
| | | RLNSVTTEDTATYYCARDLDMDYWG |
| | | QGTSVTVSS |
| mAb3-VL | SEQ ID NO: 6 | DIQMTQTTSSLSASLGDRVTISCRASQD |
| | | INNYLNWYQQKPDGTVKLLIYYISRLH |
| | | SGVPLRFSGSGSGTDFSLTISNLEPEDIA |
| | | TYYCQQYSELPLTFGGGTKLELK |
| mAb4-VH | SEQ ID NO: 7 | QAYLQQSGAELVRPGASVKMSCKASG |
| | | YTFTNYNMHWVKQTPRQGLEWIGAIY |
| | | PGNGDTSYNQKFKGKATLTVDKSSST |
| | | AYMQLSSLTSEDSAVYFCARGAMDY |
| | | WGQGTSVTVSS |
| mAb4-VL | SEQ ID NO: 8 | EIQMTQSPSSMSASLGDRITITCQATQD |
| | | IVKNLNWYQQKPGKPPSFLIYYATELA |
| | | EGVPSRFSGSGSGSDYSLTISNLESEDF |
| | | ADYYCLQSYESPYTFGGGTKLEIK |
| mAb5-VH | SEQ ID NO: 9 | EVQLQQSGTVLARPGASVKMSCKTSG |
| | | YTFTNYWMHWVKQRPGQGLEWIGAI |
| | | YPENSDTSYNQKFKDKAKVTAVTSAS |
| | | TAYMEVSSLTNEDSAVYYCTWGLAY |
| | | WGQGTLVTVSA |
| mAb5-VL | SEQ ID NO: 10 | DIVMTQSQKFMSTLVGDRVSVTCKAS |
| | | QNVGTDVAWFQKKPGQSPKALIYSAS |
| | | TRYTGVPDHFTGSGSGTDFTLTISNVQS |
| | | EDLAEYFCHQYSSSPYTFGGGTKLEIK |
| mAb6-VH | SEQ ID NO: 11 | EVQLQQSGTVLARPGASVKMSCKASG |
| | | YTFTSYWMHWVKQRPGQGLEWIGAIY |
| | | PENSDTNYNQKFKGKAKLTAVTSAST |
| | | AYMDLSSLTNEDSAVYYCANYYGSSD |
| | | SWGQGTTLTVSA |
| mAb6-VL | SEQ ID NO: 12 | ENVLTQSPAIMSATLGEKVTMSCRASS |
| | | NVKYMYWSQQKSGASPKLWIYYTSNL |
| | | ASGVPARFSGSGSGTSYSLTISSVEAED |
| | | AATYYCQQFTSSPYTFGGGTKLEIK |
| mAb7-VH | SEQ ID NO: 13 | EVQLQQSGTVLARPGASVKMSCKASG |
| | | YSFTSYWMHWVKQRPGQGLEWIGAIY |
| | | PGNSDTSYNQKFKGKAKLTAVTSAST |
| | | AYMELSSLTNEDSAVYYCTTFYDYDVI |
| | | DYWGQGTTLTVSS |
| mAb7-VL | SEQ ID NO: 14 | QIVLTQSPAIMSASPGEKVTITCSASSSV |
| | | SYMHWLQQKPGTSPKLWIYSTSNLAS |
| | | GVPARFSGSGSGTSYSLTISRMEAEDA |
| | | ATYYCQERSSSPFTFGSGTKLEIK |
| mAb8-VH | SEQ ID NO: 15 | QAYLQQSGAELVRSGASVKMSCKASG |
| | | YTFTSYNMHWVKQTPGQGLEWIGYIY |
| | | PGNGDTNYNQKFKGKATLTADTSSST |
| | | AYMQISSLTSEDSAVYFCARGAMDYW |
| | | GQGTSVTVSS |
| mAb8-VL | SEQ ID NO: 16 | DIVMTQSHKFMSTSVGDRVNITCKASQ |
| | | DVSIAVAWYQHKPGQSPKLLIYSASYR |
| | | YTGVPDRFTGSGSGTDFTFTISSVQAED |
| | | LAVYYCQQHYSSPYSFGGGTKLEIK |
| mAb9-VH | SEQ ID NO: 17 | EVQLQQSGTLLARPGASVKMSCKASG |
| | | YSFTSYWMHWVKQRPGQGLEWIGAIY |
| | | PGNSDTTYNQKFKGKARLTAVTSAST |
| | | AYMELSSLTNEDSAVYYCTIYYDYDV |
| | | DYWGQGTSVSVSS |
| mAb9-VL | SEQ ID NO: 18 | QIVLTQSPAIMSASPGEKVTMTCSASSS |
| | | VSYMYWLQQKPGTSPKLWIYSTSNLA |
| | | SGVPARFSGSGSGTSYSLTISRMEAEDA |
| | | ATYYCQQRSSYPYTFGGGANLEIK |
| mAb10-VH | SEQ ID NO: 19 | EVQLQQSGTVLARPGASVKMSCKASD |
| | | YTFTSYWMHWVKQRPGQGLEWIGAIY |
| | | PGNSDTSYNQKFKDKAKLTAVTSTSTA |
| | | YMELSSLTNEDSAVYYCSHFDVWGAG |
| | | TTVTVSS |
| mAb10-VL | SEQ ID NO: 20 | QIVLTQSPALMSASPGEKVTMTCSASS |
| | | SVNYMYWYQQKPRSSPKPWIYLTSNL |
| | | ASGVPARFSGSGSGTSYSLTISSMEAED |
| | | AATYYCQQWSSNPYTFGGGTKLEIK |
| mAb11-VH | SEQ ID NO: 21 | QAYLQQSGAELVRSGASVKMSCKASG |
| | | YTFTSYNMHWVKRTPGQGLEWIGYIY |
| | | PGNGDTNYNQKFKGKATLTADTSSST |
| | | AYMQISSLTSEDSAVYFCARGAMDYW |
| | | GQGTSVTVSS |
| mAb11-VL | SEQ ID NO: 22 | DIVMTQSHKFMSTSVGDRVSITCKASQ |
| | | DVSSNVAWYQQSPGQSPKLLIYSASYR |
| | | YTGVPDRFTGSGSGTDFTFTISSVQAED |
| | | LAVYYCQQHYSSPYTFGGGTKLEIK |
| mAb12-VH | SEQ ID NO: 23 | QVQLQQPGAELVKPGASVKMSCKASG |
| | | YSFNSYWMHWVKQRPGQGLEWIGVT |
| | | DPSGSYRSYNQKFKGKATLTVDTSSST |
| | | AYMQLSSLTSEDSAVYYCTRGDYYGT |
| | | SPYYFDYWGQGTTLTVPS |
| mAb12-VL | SEQ ID NO: 24 | DIVLTQSPASLAVSLGQRATISCRASES |
| | | VDDYGISFMNWFQQKPGQPPKLLIYA |
| | | ASNQGSGVPARFSGSGSGTDFSLNIHP |
| | | MEEDDTAMYFCQQSKEVPWTFGGGT |
| | | KLEIK |
| mAb13-VH | SEQ ID NO: 25 | EVQLQQSGTVLARPGASVKMSCKASG |
| | | YTFTSYWMHWVKQRPGQGLEWIGAIY |
| | | PENSDTDYNQKFKGKAKLTAVTSTST |
| | | AYMELSSLTNEDSAVYYCSYVDYWG |
| | | QGTTLTVSS |
| mAb13-VL | SEQ ID NO: 26 | QIVLTQSPAVMSASPGEKVTMTCSASS |
| | | SVNYMYWYQQKPRSSPKPWIYLTSNL |
| | | ASGVPARFSGSGSGTSYSLTISSMEAED |
| | | AATYYCQQWSSNPFTFGSGTKLEIK |
| mAb14-VH | SEQ ID NO: 27 | EVHLQQSGTVLARPGASVKMSCKASG |
| | | YSFTSYWMHWVKQRPGQGLEWIGAIY |
| | | PGNSDTSYNQKFKAKAKLTAVTSAST |
| | | AYMELSSLTNEDSAVYYCTIYYDYDV |
| | | DYWGQGTSVTVSS |
| mAb14-VL | SEQ ID NO: 28 | QIVLTQSPAIMSASPGEKVTITCSASSSV |
| | | SYMYWVQQKPGTSPKLWIYSTSKLAS |
| | | GVPARFSGSGSGTSYSLTISRMEAEDA |
| | | ATYYCQQRSSYPYTFGGGTKLEIK |
| mAb15-VH | SEQ ID NO: 29 | EVQLQQSGTLLARPGASVKMSCKASG |
| | | YSFTSYWMHWVKQRPGQNLEWIGAIY |
| | | PGNRDTTYNQKFKGKARLTAVTSAST |
| | | AYMELSSLTNEDSAVYYCTIYYDYDV |
| | | DYWGQGTSVSVSS |
| mAb15-VL | SEQ ID NO: 30 | QIVLTQSPAIMSASPGEKVTITCSASSSV |
| | | SYMYWLQQKPGTSPKLWIYSTSNLAS |
| | | GVPDRFSGSGSGTSYSLTISRMEAEDA |
| | | ATYYCQQRSSYPYTFGGGANLEIK |
| mAb16-VH | SEQ ID NO: 31 | EVQLQQSGTVLARPGASVKMSCKASG |
| | | STFTNYWMHWVKQRPGQGLEWIGAIY |
| | | PENGDISYNQKFKGKAKLTAVTSASTA |
| | | YMDLSSLTNEDSAVYYCTREIAYWGQ |
| | | GTLVTVSA |
| mAb16-VL | SEQ ID NO: 32 | DIVMTQSQKFMSTSVGDRVSVTCKAS |
| | | QNVGTAVAWYQKKPGQSPKALIYSAS |
| | | TRYTGVPDRFTGSGSGTDFTLTISNVQS |
| | | EDLAEYFCQQYSSSPYTFGGGTKLEIK |
| mAb17-VH | SEQ ID NO: 33 | EVQLQQSGTVLARPGASVKMSCKASG |
| | | STFTNYWMHWVKQRPGQGLEWIGAIY |
| | | PENNDVDYNQKFKGKAKLTAVTSAST |
| | | AYMELSSLTNEDSAVYYCTREITYWG |
| | | QGTSVTVSS |
| mAb17-VL | SEQ ID NO: 34 | DIVMTQSQKFMSTSVGDRVSVTCKAS |
| | | QNVGTNVAWYQQKPGQSPKALIYSAS |
| | | YRYSGVPDRFTGSGSGTDFTLTISNVQS |
| | | EDLADYFCQQHYSNPYTFGGGTKLEIK |
| mAb18-VH | SEQ ID NO: 35 | QVHVKQSGAELVRPGASVKMSCKASG |
| | | YTFTNYNMHWVKQTPRQGLEWIGAIY |
| | | PGNGDTSYNQKFKGKATLTVDKSSST |
| | | AYMQLSSLTSEDSAVYFCTRGPFDYW |
| | | GQGTTLTVSS |
| mAb18-VL | SEQ ID NO: 36 | DIVMTQSHKFMSTSVGDRVSITCKASQ |
| | | DVSTDVAWYQQKPGQSPKLLIYSASY |
| | | RYTGVPDRFTGSGSGTDFTFTISSVQAE |
| | | DLAVYYCQQHYSTPYTFGGGTKLEIK |
| mAb19-VH | SEQ ID NO: 37 | EVQLQQSGTVLARPGASVKMSCKASG |
| | | YTFTNYWMHWIIQRPGQGLEWIGSIYP |
| | | GNSDTSYNQKFMDKAKLTAVTSASTA |
| | | YLELSSLTNEDSAVYYCTKPLDSWGQ |
| | | GTTLTVSS |
| mAb19-VL | SEQ ID NO: 38 | DIQMTQSPASLSASVGETVTITCRASEN |
| | | IYSNLAWYQQKEGKSPQLLVYSATKL |
| | | ADGVPSRFSGSGSGTQFSLKIKSLQPED |
| | | FGSYYCQHFYGAPYTFGGGTKVEIK |
| mAb20-VH | SEQ ID NO: 39 | EVQLQQSGTVLARPGASVKMSCKTSG |
| | | YTFTNYYIHWIKQRPGQGLEYIGAIYPE |
| | | NSDTTYNQKFKGKAKLTAVTSASTAY |
| | | MDLNSLTDEDSAVYYCSKFDFWGQGT |
| | | TLTVSS |
| mAb20-VL | SEQ ID NO: 40 | DIVMTQSQKFMSTSVGDRVSVTCKAS |
| | | QNVGTNVAWYQQKPGQSPKALIYSAS |
| | | YRYSGVPARFTGSGSGTDFTLTISNVQS |
| | | EDLAEYYCQQHYSTPFTFGSGTKLEIK |
| mAb21-VH | SEQ ID NO: 41 | EVQLQQSGTVLARPGASVKMSCKTSG |
| | | YTFTSYWMHWVKQRPGQGLEWIGAIY |
| | | PENSDTNYNQNFKGKAKLTAVTSAST |
| | | AYMELSSLTNEDSAVYYCTTRLHYWG |
| | | TGTTVTISS |
| mAb21-VL | SEQ ID NO: 42 | DIVMTQSPASLSASVGETVTMTCRASE |
| | | NIYSNLAWYQQKQGKSPQLLVYAATN |
| | | LADGVPSRFSGSGSGTQFSLKINSLQPE |
| | | DFGSYYCQHFYGTPYTFGGGTKLEIK |
| mAb22-VH | SEQ ID NO: 43 | EVQLQQSGAELARPGASVKMSCKTSG |
| | | YTFTNYWMHWVKQRPGQGLEWIGAI |
| | | YPDNGDTDYNQKFMDKAKLTSVTSAS |
| | | TAYMELSSLTDEDSAVYFCTYAIAYW |
| | | GQGTLVTVSA |
| mAb22-VL | SEQ ID NO: 32 | DIVMTQSQKFMSTSVGDRVSVTCKAS |
| | | QNVGTAVAWYQKKPGQSPKALIYSAS |
| | | TRYTGVPDRFTGSGSGTDFTLTISNVQS |
| | | EDLAEYFCQQYSSSPYTFGGGTKLEIK |
| mAb23-VH | SEQ ID NO: 45 | EVQLQQSGTVLARPGASVKMSCKASG |
| | | YTFTIYWMHWVKQRPGQGLEWIGSIY |
| | | PENSDTNYNQKFKGKAKLTAVTSAST |
| | | AYLDLSSLTNEDSAVYYCTRYYGDYD |
| | | DWGQGTTLTVSS |
| mAb23-VL | SEQ ID NO: 46 | DIVLTQSPAIMSATLGEKVTMSCRASS |
| | | NVKYMYWYQQKSGASPKLWIYYTSN |
| | | LASGVPARFSGSGSGTSYSLTISSVEAE |
| | | DAATYYCQQFTSSPSFGGGTKLEIK |
| mAb24-VH | SEQ ID NO: 47 | EVQLQQSGAVLARPGTSVKMSCKASG |
| | | YTFTDYYIHWIKQRPGQGLEWLGAIYP |
| | | GNSDTDYNQKVRAKAKLTAVTSASTA |
| | | YMELSSLTNEDSAVYYCTRGGLDWGQ |
| | | GTTLTVSS |
| mAb24-VL | SEQ ID NO: 48 | DIVMTQSHKFMSTSVGDRVSITCKASQ |
| | | DVSTAVAWYQQKPGQSPKLLIYSASY |
| | | RYTGVPDRFTGSGSGTDFTFTISSVQAE |
| | | DLAVYYCQQHYSSPYTFGGGTKLEIK |
| mAb25-VH | SEQ ID NO: 49 | EVQLQQSGPELVKPGASVKLSCKASGS |
| | | TFTNYWMHWVKQRPGQGLEWIGAIYP |
| | | ENGDISYNQKFKGKAKLTAVTSASTA |
| | | YMDLSSLTNEDSAVYYCTREIAYWGQ |
| | | GTLVTVSS |
| mAb25-VL | SEQ ID NO: 50 | DIVMTQSPKFLPVTAEDRVTITCKASQS |
| | | VSNEVAWYQQKPGQSPKLLIYYASNR |
| | | YTGVPDRFTGSGSGTDFTFTISSVQVED |
| | | LAVYFCQQHYSSPYTFGGGTKLEIK |
| mAb26-VH | SEQ ID NO: 51 | EVQLQQSGTVLARPGASVKMSCKASG |
| | | STFTNYWMHWVKQRPGQGLEWIGAIY |
| | | PENNDVSYNQKFKGKAKLTAVTSAST |
| | | AYMELSSLTNEDSAVYYCTREITYWG |
| | | QGTLVTVSA |
| mAb26-VL | SEQ ID NO: 32 | DIVMTQSQKFMSTSVGDRVSVTCKAS |
| | | QNVGTAVAWYQKKPGQSPKALIYSAS |
| | | TRYTGVPDRFTGSGSGTDFTLTISNVQS |
| | | EDLAEYFCQQYSSSPYTFGGGTKLEIK |
| mAb27-VH | SEQ ID NO: 53 | EVQLQQSGPELVKPGASVKISCKASGY |
| | | TFTTYWMHWVKQRPGQGLEWIGAIYP |
| | | ENSDTSYNQKFKDKAKLTAVTSANTA |
| | | YMELSSLTNEDSAVYYCTRAFDWGQG |
| | | TLVTVSA |
| mAb27-VL | SEQ ID NO: 54 | DIVMTQSQKFMSTSVGDRVSVTCKAS |
| | | QNVGTNVAWYQQKPGQSPKALIYSAS |
| | | YRYSGVPDRFTGSGSGTDFTLTISSVQA |
| | | EDLAVYYCQQHYSTPYTFGGGTKLEIK |
| mAb28-VH | SEQ ID NO: 55 | QVQLQQSGSELVKPGASVKLSCKASG |
| | | YTFTSYDINWVKQRPGQGLEWIGWIYP |
| | | REIIPKYNEKFKGKATLTVDTSSSTAY |
| | | MELHSLTSEDSAVYFCAMWGSHLYAL |
| | | DYWGQGTSVTVSS |
| mAb28-VL | SEQ ID NO: 56 | DIELTQSPALMATSPGEKVTITCSVSSSI |
| | | SSSNLHWYQQKSGTSPKLWIYGTSNLA |
| | | SGVPVRFSGSGSGTSYSLTISSMEAEDA |
| | | ATYYCQQWSSYPHITFGAGTKLELK |
| mAb29-VH | SEQ ID NO: 57 | QVQLQQSGTVLARPGASVKMSCKASG |
| | | YTFTSYWMHWLKQRPGQGLEWIGAIY |
| | | PGNSDTNYNQNFKGKAKLTAVTSAST |
| | | AYMELSSLTNEDSAVYYCTRGPDGYF |
| | | EDYWGQGTTLTVSS |
| mAb29-VL | SEQ ID NO: 58 | DIVLTQSPASLAVSLGQKVTITCSVSSSI |
| | | SSSNLHWYQQKSGTSPKLWIYGTSNLA |
| | | SGVPVRFSGSGSGTSYSLTISSMEAEDA |
| | | ATYYCQQWSSYPYTFGGGTKLEIK |
| mAb1-HCDR1 | SEQ ID NO: 59 | GYTFTSYN |
| mAb1-HCDR2 | SEQ ID NO: 60 | IYPENGDT |
| mAb1-HCDR3 | SEQ ID NO: 61 | ARPFDY |
| mAb1-LCDR1 | SEQ ID NO: 62 | QSVGNN |
| mAb1-LCDR2 | SEQ ID NO: 63 | YAS |
| mAb1-LCDR3 | SEQ ID NO: 64 | QQFYSSPYT |
| mAb2-HCDR1 | SEQ ID NO: 65 | GYTFTDYW |
| mAb2-HCDR2 | SEQ ID NO: 66 | IYPGNTDT |
| mAb2-HCDR3 | SEQ ID NO: 67 | TGFNLDY |
| mAb2-LCDR1 | SEQ ID NO: 68 | QNVGTS |
| mAb2-LCDR2 | SEQ ID NO: 69 | SAS |
| mAb2-LCDR3 | SEQ ID NO: 70 | QQYGSSPYT |
| mAb3-HCDR1 | SEQ ID NO: 71 | GDSITSGYY |
| mAb3-HCDR2 | SEQ ID NO: 72 | ISYEGSN |
| mAb3-HCDR3 | SEQ ID NO: 73 | ARDLDMDY |
| mAb3-LCDR1 | SEQ ID NO: 74 | QDINNY |
| mAb3-LCDR2 | SEQ ID NO: 75 | YIS |
| mAb3-LCDR3 | SEQ ID NO: 76 | QQYSELPLT |
| mAb4-HCDR1 | SEQ ID NO: 77 | GYTFTNYN |
| mAb4-HCDR2 | SEQ ID NO: 78 | IYPGNGDT |
| mAb4-HCDR3 | SEQ ID NO: 79 | ARGAMDY |
| mAb4-LCDR1 | SEQ ID NO: 80 | QDIVKN |
| mAb4-LCDR2 | SEQ ID NO: 81 | YAT |
| mAb4-LCDR3 | SEQ ID NO: 82 | LQSYESPYT |
| mAb5-HCDR1 | SEQ ID NO: 83 | GYTFTNYW |
| mAb5-HCDR2 | SEQ ID NO: 84 | IYPENSDT |
| mAb5-HCDR3 | SEQ ID NO: 85 | TWGLAY |
| mAb5-LCDR1 | SEQ ID NO: 86 | QNVGTD |
| mAb5-LCDR2 | SEQ ID NO: 69 | SAS |
| mAb5-LCDR3 | SEQ ID NO: 87 | HQYSSSPYT |
| mAb6-HCDR1 | SEQ ID NO: 88 | GYTFTSYW |
| mAb6-HCDR2 | SEQ ID NO: 84 | IYPENSDT |
| mAb6-HCDR3 | SEQ ID NO: 89 | ANYYGSSDS |
| mAb6-LCDR1 | SEQ ID NO: 90 | SNVKY |
| mAb6-LCDR2 | SEQ ID NO: 91 | YTS |
| mAb6-LCDR3 | SEQ ID NO: 92 | QQFTSSPYT |
| mAb7-HCDR1 | SEQ ID NO: 93 | GYSFTSYW |
| mAb7-HCDR2 | SEQ ID NO: 94 | IYPGNSDT |
| mAb7-HCDR3 | SEQ ID NO: 95 | TTFYDYDVIDY |
| mAb7-LCDR1 | SEQ ID NO: 96 | SSVSY |
| mAb7-LCDR2 | SEQ ID NO: 97 | STS |
| mAb7-LCDR3 | SEQ ID NO: 98 | QERSSSPFT |
| mAb8-HCDR1 | SEQ ID NO: 59 | GYTFTSYN |
| mAb8-HCDR2 | SEQ ID NO: 78 | IYPGNGDT |
| mAb8-HCDR3 | SEQ ID NO: 79 | ARGAMDY |
| mAb8-LCDR1 | SEQ ID NO: 99 | QDVSIA |
| mAb8-LCDR2 | SEQ ID NO: 69 | SAS |
| mAb8-LCDR3 | SEQ ID NO: 100 | QQHYSSPYS |
| mAb9-HCDR1 | SEQ ID NO: 93 | GYSFTSYW |
| mAb9-HCDR2 | SEQ ID NO: 94 | IYPGNSDT |
| mAb9-HCDR3 | SEQ ID NO: 101 | TIYYDYDVDY |
| mAb9-LCDR1 | SEQ ID NO: 96 | SSVSY |
| mAb9-LCDR2 | SEQ ID NO: 97 | STS |
| mAb9-LCDR3 | SEQ ID NO: 102 | QQRSSYPYT |
| mAb10-HCDR1 | SEQ ID NO: 103 | DYTFTSYW |
| mAb10-HCDR2 | SEQ ID NO: 94 | IYPGNSDT |
| mAb10-HCDR3 | SEQ ID NO: 104 | SHFDV |
| mAb10-LCDR1 | SEQ ID NO: 105 | SSVNY |
| mAb10-LCDR2 | SEQ ID NO: 106 | LTS |
| mAb10-LCDR3 | SEQ ID NO: 107 | QQWSSNPYT |
| mAb11-HCDR1 | SEQ ID NO: 59 | GYTFTSYN |
| mAb11-HCDR2 | SEQ ID NO: 78 | IYPGNGDT |
| mAb11-HCDR3 | SEQ ID NO: 79 | ARGAMDY |
| mAb11-LCDR1 | SEQ ID NO: 108 | QDVSSN |
| mAb11-LCDR2 | SEQ ID NO: 69 | SAS |
| mAb11-LCDR3 | SEQ ID NO: 109 | QQHYSSPYT |
| mAb12-HCDR1 | SEQ ID NO: 110 | GYSFNSYW |
| mAb12-HCDR2 | SEQ ID NO: 111 | TDPSGSYR |
| mAb12-HCDR3 | SEQ ID NO: 112 | TRGDYYGTSPYYFDY |
| mAb12-LCDR1 | SEQ ID NO: 113 | ESVDDYGISF |
| mAb12-LCDR2 | SEQ ID NO: 114 | AAS |
| mAb12-LCDR3 | SEQ ID NO: 115 | QQSKEVPWT |
| mAb13-HCDR1 | SEQ ID NO: 88 | GYTFTSYW |
| mAb13-HCDR2 | SEQ ID NO: 84 | IYPENSDT |
| mAb13-HCDR3 | SEQ ID NO: 116 | SYVDY |
| mAb13-LCDR1 | SEQ ID NO: 105 | SSVNY |
| mAb13-LCDR2 | SEQ ID NO: 106 | LTS |
| mAb13-LCDR3 | SEQ ID NO: 117 | QQWSSNPFT |
| mAb14-HCDR1 | SEQ ID NO: 93 | GYSFTSYW |
| mAb14-HCDR2 | SEQ ID NO: 94 | IYPGNSDT |
| mAb14-HCDR3 | SEQ ID NO: 101 | TIYYDYDVDY |
| mAb14-LCDR1 | SEQ ID NO: 96 | SSVSY |
| mAb14-LCDR2 | SEQ ID NO: 97 | STS |
| mAb14-LCDR3 | SEQ ID NO: 102 | QQRSSYPYT |
| mAb15-HCDR1 | SEQ ID NO: 93 | GYSFTSYW |
| mAb15-HCDR2 | SEQ ID NO: 118 | IYPGNRDT |
| mAb15-HCDR3 | SEQ ID NO: 101 | TIYYDYDVDY |
| mAb15-LCDR1 | SEQ ID NO: 96 | SSVSY |
| mAb15-LCDR2 | SEQ ID NO: 97 | STS |
| mAb15-LCDR3 | SEQ ID NO: 102 | QQRSSYPYT |
| mAb16-HCDR1 | SEQ ID NO: 119 | GSTFTNYW |
| mAb16-HCDR2 | SEQ ID NO: 120 | IYPENGDI |
| mAb16-HCDR3 | SEQ ID NO: 121 | TREIAY |
| mAb16-LCDR1 | SEQ ID NO: 122 | QNVGTA |
| mAb16-LCDR2 | SEQ ID NO: 69 | SAS |
| mAb16-LCDR3 | SEQ ID NO: 123 | QQYSSSPYT |
| mAb17-HCDR1 | SEQ ID NO: 119 | GSTFTNYW |
| mAb17-HCDR2 | SEQ ID NO: 124 | IYPENNDV |
| mAb17-HCDR3 | SEQ ID NO: 125 | TREITY |
| mAb17-LCDR1 | SEQ ID NO: 126 | QNVGTN |
| mAb17-LCDR2 | SEQ ID NO: 69 | SAS |
| mAb17-LCDR3 | SEQ ID NO: 127 | QQHYSNPYT |
| mAb18-HCDR1 | SEQ ID NO: 77 | GYTFTNYN |
| mAb18-HCDR2 | SEQ ID NO: 78 | IYPGNGDT |
| mAb18-HCDR3 | SEQ ID NO: 128 | TRGPFDY |
| mAb18-LCDR1 | SEQ ID NO: 129 | QDVSTD |
| mAb18-LCDR2 | SEQ ID NO: 69 | SAS |
| mAb18-LCDR3 | SEQ ID NO: 130 | QQHYSTPYT |
| mAb19-HCDR1 | SEQ ID NO: 83 | GYTFTNYW |
| mAb19-HCDR2 | SEQ ID NO: 94 | IYPGNSDT |
| mAb19-HCDR3 | SEQ ID NO: 131 | TKPLDS |
| mAb19-LCDR1 | SEQ ID NO: 132 | ENIYSN |
| mAb19-LCDR2 | SEQ ID NO: 133 | SAT |
| mAb19-LCDR3 | SEQ ID NO: 134 | QHFYGAPYT |
| mAb20-HCDR1 | SEQ ID NO: 135 | GYTFTNYY |
| mAb20-HCDR2 | SEQ ID NO: 84 | IYPENSDT |
| mAb20-HCDR3 | SEQ ID NO: 136 | SKFDF |
| mAb20-LCDR1 | SEQ ID NO: 126 | QNVGTN |
| mAb20-LCDR2 | SEQ ID NO: 69 | SAS |
| mAb20-LCDR3 | SEQ ID NO: 137 | QQHYSTPFT |
| mAb21-HCDR1 | SEQ ID NO: 88 | GYTFTSYW |
| mAb21-HCDR2 | SEQ ID NO: 84 | IYPENSDT |
| mAb21-HCDR3 | SEQ ID NO: 138 | TTRLHY |
| mAb21-LCDR1 | SEQ ID NO: 132 | ENIYSN |
| mAb21-LCDR2 | SEQ ID NO: 139 | AAT |
| mAb21-LCDR3 | SEQ ID NO: 140 | QHFYGTPYT |
| mAb22-HCDR1 | SEQ ID NO: 83 | GYTFTNYW |
| mAb22-HCDR2 | SEQ ID NO: 141 | IYPDNGDT |
| mAb22-HCDR3 | SEQ ID NO: 142 | TYALAY |
| mAb22-LCDR1 | SEQ ID NO: 122 | QNVGTA |
| mAb22-LCDR2 | SEQ ID NO: 69 | SAS |
| mAb22-LCDR3 | SEQ ID NO: 123 | QQYSSSPYT |
| mAb23-HCDR1 | SEQ ID NO: 143 | GYTFTIYW |
| mAb23-HCDR2 | SEQ ID NO: 84 | IYPENSDT |
| mAb23-HCDR3 | SEQ ID NO: 144 | TRYYGDYDD |
| mAb23-LCDR1 | SEQ ID NO: 90 | SNVKY |
| mAb23-LCDR2 | SEQ ID NO: 91 | YTS |
| mAb23-LCDR3 | SEQ ID NO: 145 | QQFTSSPS |
| mAb24-HCDR1 | SEQ ID NO: 146 | GYTFTDYY |
| mAb24-HCDR2 | SEQ ID NO: 94 | IYPGNSDT |
| mAb24-HCDR3 | SEQ ID NO: 147 | TRGGLD |
| mAb24-LCDR1 | SEQ ID NO: 148 | QDVSTA |
| mAb24-LCDR2 | SEQ ID NO: 69 | SAS |
| mAb24-LCDR3 | SEQ ID NO: 109 | QQHYSSPYT |
| mAb25-HCDR1 | SEQ ID NO: 119 | GSTFTNYW |
| mAb25-HCDR2 | SEQ ID NO: 120 | IYPENGDI |
| mAb25-HCDR3 | SEQ ID NO: 121 | TREIAY |
| mAb25-LCDR1 | SEQ ID NO: 149 | QSVSNE |
| mAb25-LCDR2 | SEQ ID NO: 63 | YAS |
| mAb25-LCDR3 | SEQ ID NO: 109 | QQHYSSPYT |
| mAb26-HCDR1 | SEQ ID NO: 119 | GSTFTNYW |
| mAb26-HCDR2 | SEQ ID NO: 124 | IYPENNDV |
| mAb26-HCDR3 | SEQ ID NO: 125 | TREITY |
| mAb26-LCDR1 | SEQ ID NO: 122 | QNVGTA |
| mAb26-LCDR2 | SEQ ID NO: 69 | SAS |
| mAb26-LCDR3 | SEQ ID NO: 123 | QQYSSSPYT |
| mAb27-HCDR1 | SEQ ID NO: 150 | GYTFTTYW |
| mAb27-HCDR2 | SEQ ID NO: 84 | IYPENSDT |
| mAb27-HCDR3 | SEQ ID NO: 151 | TRAFD |
| mAb27-LCDR1 | SEQ ID NO: 126 | QNVGTN |
| mAb27-LCDR2 | SEQ ID NO: 69 | SAS |
| mAb27-LCDR3 | SEQ ID NO: 130 | QQHYSTPYT |
| mAb28-HCDR1 | SEQ ID NO: 152 | GYTFTSYD |
| mAb28-HCDR2 | SEQ ID NO: 153 | IYPREIIP |
| mAb28-HCDR3 | SEQ ID NO: 154 | AMWGSHLYALDY |
| mAb28-LCDR1 | SEQ ID NO: 155 | SSISSSN |
| mAb28-LCDR2 | SEQ ID NO: 156 | GTS |
| mAb28-LCDR3 | SEQ ID NO: 157 | QQWSSYPHIT |
| mAb29-HCDR1 | SEQ ID NO: 88 | GYTFTSYW |
| mAb29-HCDR2 | SEQ ID NO: 94 | IYPGNSDT |
| mAb29-HCDR3 | SEQ ID NO: 158 | TRGPDGYFEDY |
| mAb29-LCDR1 | SEQ ID NO: 155 | SSISSSN |
| mAb29-LCDR2 | SEQ ID NO: 156 | GTS |
| mAb29-LCDR3 | SEQ ID NO: 159 | QQWSSYPYT |
| mAb1-HFR1 | SEQ ID NO: 160 | QAYLQQSGAELVRPGASVKMSCKAS |
| mAb1-HFR2 | SEQ ID NO: 161 | MHWVKQTPRQGLEWIGS |
| mAb1-HFR3 | SEQ ID NO: 162 | SYNQKFRDKATLTVDKSSSTAYMQLS |
| | | SLTSEDSAVYFC |
| mAb1-HFR4 | SEQ ID NO: 163 | WGQGTTLTVSS |
| mAb1-LFR1 | SEQ ID NO: 164 | SIVMTQTPKFLPISAGDRVTMTCKAS |
| mAb1-LFR2 | SEQ ID NO: 165 | VAWYQQKPGQSPKLLIY |
| mAb1-LFR3 | SEQ ID NO: 166 | NRYTGVPDRFTGSGSGTDFTFTISSVQV |
| | | EDLAVYFC |
| mAb1-LFR4 | SEQ ID NO: 167 | FGSGTKLEIK |
| mAb2-HFR1 | SEQ ID NO: 168 | EVQLQQSGTVLARPGASVKMSCRAS |
| mAb2-HFR2 | SEQ ID NO: 169 | MHWVKQRPGQGLEWIGA |
| mAb2-HFR3 | SEQ ID NO: 170 | DYNQKFKGKAKLTAVTSASSAYMELS |
| | | SLTNEDSAVYYC |
| mAb2-HFR4 | SEQ ID NO: 163 | WGQGTTLTVSS |
| mAb2-LFR1 | SEQ ID NO: 171 | DIVMTQSQKFMSTSVGDRVSVTCKAS |
| mAb2-LFR2 | SEQ ID NO: 172 | VAWYQKKPGQSPKVVIY |
| mAb2-LFR3 | SEQ ID NO: 173 | TRYTGVPDRFTGSGLGTDFTLTISNVQS |
| | | EDLAEYFC |
| mAb2-LFR4 | SEQ ID NO: 174 | FGGGTKLEIK |
| mAb3-HFR1 | SEQ ID NO: 175 | DVQLQESGPGLVKPSQSLSLTCSVT |
| mAb3-HFR2 | SEQ ID NO: 176 | WNWIRQFPGNKLEWMGY |
| mAb3-HFR3 | SEQ ID NO: 177 | NYNPSLKNRISITRDTSKNQFFLRLNSV |
| | | TTEDTATYYC |
| mAb3-HFR4 | SEQ ID NO: 178 | WGQGTSVTVSS |
| mAb3-LFR1 | SEQ ID NO: 179 | DIQMTQTTSSLSASLGDRVTISCRAS |
| mAb3-LFR2 | SEQ ID NO: 180 | LNWYQQKPDGTVKLLIY |
| mAb3-LFR3 | SEQ ID NO: 181 | RLHSGVPLRFSGSGSGTDFSLTISNLEP |
| | | EDIATYYC |
| mAb3-LFR4 | SEQ ID NO: 182 | FGGGTKLELK |
| mAb4-HFR1 | SEQ ID NO: 160 | QAYLQQSGAELVRPGASVKMSCKAS |
| mAb4-HFR2 | SEQ ID NO: 183 | MHWVKQTPRQGLEWIGA |
| mAb4-HFR3 | SEQ ID NO: 184 | SYNQKFKGKATLTVDKSSSTAYMQLS |
| | | SLTSEDSAVYFC |
| mAb4-HFR4 | SEQ ID NO: 178 | WGQGTSVTVSS |
| mAb4-LFR1 | SEQ ID NO: 185 | EIQMTQSPSSMSASLGDRITITCQAT |
| mAb4-LFR2 | SEQ ID NO: 186 | LNWYQQKPGKPPSFLIY |
| mAb4-LFR3 | SEQ ID NO: 187 | ELAEGVPSRFSGSGSGSDYSLTISNLES |
| | | EDFADYYC |
| mAb4-LFR4 | SEQ ID NO: 174 | FGGGTKLEIK |
| mAb5-HFR1 | SEQ ID NO: 188 | EVQLQQSGTVLARPGASVKMSCKTS |
| mAb5-HFR2 | SEQ ID NO: 169 | MHWVKQRPGQGLEWIGA |
| mAb5-HFR3 | SEQ ID NO: 189 | SYNQKFKDKAKVTAVTSASTAYMEVS |
| | | SLTNEDSAVYYC |
| mAb5-HFR4 | SEQ ID NO: 190 | WGQGTLVTVSA |
| mAb5-LFR1 | SEQ ID NO: 191 | DIVMTQSQKFMSTLVGDRVSVTCKAS |
| mAb5-LFR2 | SEQ ID NO: 192 | VAWFQKKPGQSPKALIY |
| mAb5-LFR3 | SEQ ID NO: 193 | TRYTGVPDHFTGSGSGTDFTLTISNVQS |
| | | EDLAEYFC |
| mAb5-LFR4 | SEQ ID NO: 174 | FGGGTKLEIK |
| mAb6-HFR1 | SEQ ID NO: 194 | EVQLQQSGTVLARPGASVKMSCKAS |
| mAb6-HFR2 | SEQ ID NO: 169 | MHWVKQRPGQGLEWIGA |
| mAb6-HFR3 | SEQ ID NO: 195 | NYNQKFKGKAKLTAVTSASTAYMDLS |
| | | SLTNEDSAVYYC |
| mAb6-HFR4 | SEQ ID NO: 196 | WGQGTTLTVSA |
| mAb6-LFR1 | SEQ ID NO: 197 | ENVLTQSPAIMSATLGEKVTMSCRAS |
| mAb6-LFR2 | SEQ ID NO: 198 | MYWSQQKSGASPKLWIY |
| mAb6-LFR3 | SEQ ID NO: 199 | NLASGVPARFSGSGSGTSYSLTISSVEA |
| | | EDAATYYC |
| mAb6-LFR4 | SEQ ID NO: 174 | FGGGTKLEIK |
| mAb7-HFR1 | SEQ ID NO: 194 | EVQLQQSGTVLARPGASVKMSCKAS |
| mAb7-HFR2 | SEQ ID NO: 169 | MHWVKQRPGQGLEWIGA |
| mAb7-HFR3 | SEQ ID NO: 200 | SYNQKFKGKAKLTAVTSASTAYMELS |
| | | SLTNEDSAVYYC |
| mAb7-HFR4 | SEQ ID NO: 163 | WGQGTTLTVSS |
| mAb7-LFR1 | SEQ ID NO: 201 | QIVLTQSPAIMSASPGEKVTITCSAS |
| mAb7-LFR2 | SEQ ID NO: 202 | MHWLQQKPGTSPKLWIY |
| mAb7-LFR3 | SEQ ID NO: 203 | NLASGVPARFSGSGSGTSYSLTISRMEA |
| | | EDAATYYC |
| mAb7-LFR4 | SEQ ID NO: 167 | FGSGTKLEIK |
| mAb8-HFR1 | SEQ ID NO: 204 | QAYLQQSGAELVRSGASVKMSCKAS |
| mAb8-HFR2 | SEQ ID NO: 205 | MHWVKQTPGQGLEWIGY |
| mAb8-HFR3 | SEQ ID NO: 206 | NYNQKFKGKATLTADTSSSTAYMQISS |
| | | LTSEDSAVYFC |
| mAb8-HFR4 | SEQ ID NO: 178 | WGQGTSVTVSS |
| mAb8-LFR1 | SEQ ID NO: 207 | DIVMTQSHKFMSTSVGDRVNITCKAS |
| mAb8-LFR2 | SEQ ID NO: 208 | VAWYQHKPGQSPKLLIY |
| mAb8-LFR3 | SEQ ID NO: 209 | YRYTGVPDRFTGSGSGTDFTFTISSVQA |
| | | EDLAVYYC |
| mAb8-LFR4 | SEQ ID NO: 174 | FGGGTKLEIK |
| mAb9-HFR1 | SEQ ID NO: 210 | EVQLQQSGTLLARPGASVKMSCKAS |
| mAb9-HFR2 | SEQ ID NO: 169 | MHWVKQRPGQGLEWIGA |
| mAb9-HFR3 | SEQ ID NO: 211 | TYNQKFKGKARLTAVTSASTAYMELS |
| | | SLTNEDSAVYYC |
| mAb9-HFR4 | SEQ ID NO: 212 | WGQGTSVSVSS |
| mAb9-LFR1 | SEQ ID NO: 213 | QIVLTQSPAIMSASPGEKVTMTCSAS |
| mAb9-LFR2 | SEQ ID NO: 214 | MYWLQQKPGTSPKLWIY |
| mAb9-LFR3 | SEQ ID NO: 203 | NLASGVPARFSGSGSGTSYSLTISRMEA |
| | | EDAATYYC |
| mAb9-LFR4 | SEQ ID NO: 215 | FGGGANLEIK |
| mAb10-HFR1 | SEQ ID NO: 194 | EVQLQQSGTVLARPGASVKMSCKAS |
| mAb10-HFR2 | SEQ ID NO: 169 | MHWVKQRPGQGLEWIGA |
| mAb10-HFR3 | SEQ ID NO: 216 | SYNQKFKDKAKLTAVTSTSTAYMELS |
| | | SLTNEDSAVYYC |
| mAb10-HFR4 | SEQ ID NO: 217 | WGAGTTVTVSS |
| mAb10-LFR1 | SEQ ID NO: 218 | QIVLTQSPALMSASPGEKVTMTCSAS |
| mAb10-LFR2 | SEQ ID NO: 219 | MYWYQQKPRSSPKPWIY |
| mAb10-LFR3 | SEQ ID NO: 220 | NLASGVPARFSGSGSGTSYSLTISSMEA |
| | | EDAATYYC |
| mAb10-LFR4 | SEQ ID NO: 174 | FGGGTKLEIK |
| mAb11-HFR1 | SEQ ID NO: 204 | QAYLQQSGAELVRSGASVKMSCKAS |
| mAb11-HFR2 | SEQ ID NO: 221 | MHWVKRTPGQGLEWIGY |
| mAb11-HFR3 | SEQ ID NO: 206 | NYNQKFKGKATLTADTSSSTAYMQISS |
| | | LTSEDSAVYFC |
| mAb11-HFR4 | SEQ ID NO: 178 | WGQGTSVTVSS |
| mAb11-LFR1 | SEQ ID NO: 222 | DIVMTQSHKFMSTSVGDRVSITCKAS |
| mAb11-LFR2 | SEQ ID NO: 223 | VAWYQQSPGQSPKLLIY |
| mAb11-LFR3 | SEQ ID NO: 209 | YRYTGVPDRFTGSGSGTDFTFTISSVQA |
| | | EDLAVYYC |
| mAb11-LFR4 | SEQ ID NO: 174 | FGGGTKLEIK |
| mAb12-HFR1 | SEQ ID NO: 224 | QVQLQQPGAELVKPGASVKMSCKAS |
| mAb12-HFR2 | SEQ ID NO: 225 | MHWVKQRPGQGLEWIGV |
| mAb12-HFR3 | SEQ ID NO: 226 | SYNQKFKGKATLTVDTSSSTAYMQLSS |
| | | LTSEDSAVYYC |
| mAb12-HFR4 | SEQ ID NO: 227 | WGQGTTLTVPS |
| mAb12-LFR1 | SEQ ID NO: 228 | DIVLTQSPASLAVSLGQRATISCRAS |
| mAb12-LFR2 | SEQ ID NO: 229 | MNWFQQKPGQPPKLLIY |
| mAb12-LFR3 | SEQ ID NO: 230 | NQGSGVPARFSGSGSGTDFSLNIHPME |
| | | EDDTAMYFC |
| mAb12-LFR4 | SEQ ID NO: 174 | FGGGTKLEIK |
| mAb13-HFR1 | SEQ ID NO: 194 | EVQLQQSGTVLARPGASVKMSCKAS |
| mAb13-HFR2 | SEQ ID NO: 169 | MHWVKQRPGQGLEWIGA |
| mAb13-HFR3 | SEQ ID NO: 231 | DYNQKFKGKAKLTAVTSTSTAYMELS |
| | | SLTNEDSAVYYC |
| mAb13-HFR4 | SEQ ID NO: 163 | WGQGTTLTVSS |
| mAb13-LFR1 | SEQ ID NO: 232 | QIVLTQSPAVMSASPGEKVTMTCSAS |
| mAb13-LFR2 | SEQ ID NO: 219 | MYWYQQKPRSSPKPWIY |
| mAb13-LFR3 | SEQ ID NO: 220 | NLASGVPARFSGSGSGTSYSLTISSMEA |
| | | EDAATYYC |
| mAb13-LFR4 | SEQ ID NO: 167 | FGSGTKLEIK |
| mAb14-HFR1 | SEQ ID NO: 233 | EVHLQQSGTVLARPGASVKMSCKAS |
| mAb14-HFR2 | SEQ ID NO: 169 | MHWVKQRPGQGLEWIGA |
| mAb14-HFR3 | SEQ ID NO: 234 | SYNQKFKAKAKLTAVTSASTAYMELS |
| | | SLTNEDSAVYYC |
| mAb14-HFR4 | SEQ ID NO: 178 | WGQGTSVTVSS |
| mAb14-LFR1 | SEQ ID NO: 201 | QIVLTQSPAIMSASPGEKVTITCSAS |
| mAb14-LFR2 | SEQ ID NO: 235 | MYWVQQKPGTSPKLWIY |
| mAb14-LFR3 | SEQ ID NO: 236 | KLASGVPARFSGSGSGTSYSLTISRMEA |
| | | EDAATYYC |
| mAb14-LFR4 | SEQ ID NO: 174 | FGGGTKLEIK |
| mAb15-HFR1 | SEQ ID NO: 210 | EVQLQQSGTLLARPGASVKMSCKAS |
| mAb15-HFR2 | SEQ ID NO: 237 | MHWVKQRPGQNLEWIGA |
| mAb15-HFR3 | SEQ ID NO: 211 | TYNQKFKGKARLTAVTSASTAYMELS |
| | | SLTNEDSAVYYC |
| mAb15-HFR4 | SEQ ID NO: 212 | WGQGTSVSVSS |
| mAb15-LFR1 | SEQ ID NO: 201 | QIVLTQSPAIMSASPGEKVTITCSAS |
| mAb15-LFR2 | SEQ ID NO: 214 | MYWLQQKPGTSPKLWIY |
| mAb15-LFR3 | SEQ ID NO: 238 | NLASGVPDRFSGSGSGTSYSLTISRMEA |
| | | EDAATYYC |
| mAb15-LFR4 | SEQ ID NO: 215 | FGGGANLEIK |
| mAb16-HFR1 | SEQ ID NO: 194 | EVQLQQSGTVLARPGASVKMSCKAS |
| mAb16-HFR2 | SEQ ID NO: 169 | MHWVKQRPGQGLEWIGA |
| mAb16-HFR3 | SEQ ID NO: 239 | SYNQKFKGKAKLTAVTSASTAYMDLS |
| | | SLTNEDSAVYYC |
| mAb16-HFR4 | SEQ ID NO: 190 | WGQGTLVTVSA |
| mAb16-LFR1 | SEQ ID NO: 171 | DIVMTQSQKFMSTSVGDRVSVTCKAS |
| mAb16-LFR2 | SEQ ID NO: 240 | VAWYQKKPGQSPKALIY |
| mAb16-LFR3 | SEQ ID NO: 241 | TRYTGVPDRFTGSGSGTDFTLTISNVQS |
| | | EDLAEYFC |
| mAb16-LFR4 | SEQ ID NO: 174 | FGGGTKLEIK |
| mAb17-HFR1 | SEQ ID NO: 194 | EVQLQQSGTVLARPGASVKMSCKAS |
| mAb17-HFR2 | SEQ ID NO: 169 | MHWVKQRPGQGLEWIGA |
| mAb17-HFR3 | SEQ ID NO: 242 | DYNQKFKGKAKLTAVTSASTAYMELS |
| | | SLTNEDSAVYYC |
| mAb17-HFR4 | SEQ ID NO: 178 | WGQGTSVTVSS |
| mAb17-LFR1 | SEQ ID NO: 171 | DIVMTQSQKFMSTSVGDRVSVTCKAS |
| mAb17-LFR2 | SEQ ID NO: 243 | VAWYQQKPGQSPKALIY |
| mAb17-LFR3 | SEQ ID NO: 244 | YRYSGVPDRFTGSGSGTDFTLTISNVQS |
| | | EDLADYFC |
| mAb17-LFR4 | SEQ ID NO: 174 | FGGGTKLEIK |
| mAb18-HFR1 | SEQ ID NO: 284 | QVHVKQSGAELVRPGASVKMSCKAS |
| mAb18-HFR2 | SEQ ID NO: 183 | MHWVKQTPRQGLEWIGA |
| mAb18-HFR3 | SEQ ID NO: 184 | SYNQKFKGKATLTVDKSSSTAYMQLS |
| | | SLTSEDSAVYFC |
| mAb18-HFR4 | SEQ ID NO: 163 | WGQGTTLTVSS |
| mAb18-LFR1 | SEQ ID NO: 222 | DIVMTQSHKFMSTSVGDRVSITCKAS |
| mAb18-LFR2 | SEQ ID NO: 165 | VAWYQQKPGQSPKLLIY |
| mAb18-LFR3 | SEQ ID NO: 209 | YRYTGVPDRFTGSGSGTDFTFTISSVQA |
| | | EDLAVYYC |
| mAb18-LFR4 | SEQ ID NO: 174 | FGGGTKLEIK |
| mAb19-HFR1 | SEQ ID NO: 194 | EVQLQQSGTVLARPGASVKMSCKAS |
| mAb19-HFR2 | SEQ ID NO: 245 | MHWIIQRPGQGLEWIGS |
| mAb19-HFR3 | SEQ ID NO: 246 | SYNQKFMDKAKLTAVTSASTAYLELS |
| | | SLTNEDSAVYYC |
| mAb19-HFR4 | SEQ ID NO: 163 | WGQGTTLTVSS |
| mAb19-LFR1 | SEQ ID NO: 247 | DIQMTQSPASLSASVGETVTITCRAS |
| mAb19-LFR2 | SEQ ID NO: 248 | LAWYQQKEGKSPQLLVY |
| mAb19-LFR3 | SEQ ID NO: 249 | KLADGVPSRFSGSGSGTQFSLKIKSLQP |
| | | EDFGSYYC |
| mAb19-LFR4 | SEQ ID NO: 250 | FGGGTKVEIK |
| mAb20-HFR1 | SEQ ID NO: 188 | EVQLQQSGTVLARPGASVKMSCKTS |
| mAb20-HFR2 | SEQ ID NO: 251 | IHWIKQRPGQGLEYIGA |
| mAb20-HFR3 | SEQ ID NO: 252 | TYNQKFKGKAKLTAVTSASTAYMDLN |
| | | SLTDEDSAVYYC |
| mAb20-HFR4 | SEQ ID NO: 163 | WGQGTTLTVSS |
| mAb20-LFR1 | SEQ ID NO: 171 | DIVMTQSQKFMSTSVGDRVSVTCKAS |
| mAb20-LFR2 | SEQ ID NO: 243 | VAWYQQKPGQSPKALIY |
| mAb20-LFR3 | SEQ ID NO: 253 | YRYSGVPARFTGSGSGTDFTLTISNVQS |
| | | EDLAEYYC |
| mAb20-LFR4 | SEQ ID NO: 167 | FGSGTKLEIK |
| mAb21-HFR1 | SEQ ID NO: 188 | EVQLQQSGTVLARPGASVKMSCKTS |
| mAb21-HFR2 | SEQ ID NO: 169 | MHWVKQRPGQGLEWIGA |
| mAb21-HFR3 | SEQ ID NO: 254 | NYNQNFKGKAKLTAVTSASTAYMELS |
| | | SLTNEDSAVYYC |
| mAb21-HFR4 | SEQ ID NO: 255 | WGTGTTVTISS |
| mAb21-LFR1 | SEQ ID NO: 256 | DIVMTQSPASLSASVGETVTMTCRAS |
| mAb21-LFR2 | SEQ ID NO: 257 | LAWYQQKQGKSPQLLVY |
| mAb21-LFR3 | SEQ ID NO: 258 | NLADGVPSRFSGSGSGTQFSLKINSLQP |
| | | EDFGSYYC |
| mAb21-LFR4 | SEQ ID NO: 174 | FGGGTKLEIK |
| mAb22-HFR1 | SEQ ID NO: 259 | EVQLQQSGAELARPGASVKMSCKTS |
| mAb22-HFR2 | SEQ ID NO: 169 | MHWVKQRPGQGLEWIGA |
| mAb22-HFR3 | SEQ ID NO: 260 | DYNQKFMDKAKLTSVTSASTAYMELS |
| | | SLTDEDSAVYFC |
| mAb22-HFR4 | SEQ ID NO: 190 | WGQGTLVTVSA |
| mAb22-LFR1 | SEQ ID NO: 171 | DIVMTQSQKFMSTSVGDRVSVTCKAS |
| mAb22-LFR2 | SEQ ID NO: 240 | VAWYQKKPGQSPKALIY |
| mAb22-LFR3 | SEQ ID NO: 241 | TRYTGVPDRFTGSGSGTDFTLTISNVQS |
| | | EDLAEYFC |
| mAb22-LFR4 | SEQ ID NO: 174 | FGGGTKLEIK |
| mAb23-HFR1 | SEQ ID NO: 194 | EVQLQQSGTVLARPGASVKMSCKAS |
| mAb23-HFR2 | SEQ ID NO: 261 | MHWVKQRPGQGLEWIGS |
| mAb23-HFR3 | SEQ ID NO: 262 | NYNQKFKGKAKLTAVTSASTAYLDLS |
| | | SLTNEDSAVYYC |
| mAb23-HFR4 | SEQ ID NO: 163 | WGQGTTLTVSS |
| mAb23-LFR1 | SEQ ID NO: 263 | DIVLTQSPAIMSATLGEKVTMSCRAS |
| mAb23-LFR2 | SEQ ID NO: 264 | MYWYQQKSGASPKLWIY |
| mAb23-LFR3 | SEQ ID NO: 199 | NLASGVPARFSGSGSGTSYSLTISSVEA |
| | | EDAATYYC |
| mAb23-LFR4 | SEQ ID NO: 174 | FGGGTKLEIK |
| mAb24-HFR1 | SEQ ID NO: 265 | EVQLQQSGAVLARPGTSVKMSCKAS |
| mAb24-HFR2 | SEQ ID NO: 266 | IHWIKQRPGQGLEWLGA |
| mAb24-HFR3 | SEQ ID NO: 267 | DYNQKVRAKAKLTAVTSASTAYMELS |
| | | SLTNEDSAVYYC |
| mAb24-HFR4 | SEQ ID NO: 163 | WGQGTTLTVSS |
| mAb24-LFR1 | SEQ ID NO: 222 | DIVMTQSHKFMSTSVGDRVSITCKAS |
| mAb24-LFR2 | SEQ ID NO: 165 | VAWYQQKPGQSPKLLIY |
| mAb24-LFR3 | SEQ ID NO: 209 | YRYTGVPDRFTGSGSGTDFTFTISSVQA |
| | | EDLAVYYC |
| mAb24-LFR4 | SEQ ID NO: 174 | FGGGTKLEIK |
| mAb25-HFR1 | SEQ ID NO: 268 | EVQLQQSGPELVKPGASVKLSCKAS |
| mAb25-HFR2 | SEQ ID NO: 169 | MHWVKQRPGQGLEWIGA |
| mAb25-HFR3 | SEQ ID NO: 239 | SYNQKFKGKAKLTAVTSASTAYMDLS |
| | | SLTNEDSAVYYC |
| mAb25-HFR4 | SEQ ID NO: 269 | WGQGTLVTVSS |
| mAb25-LFR1 | SEQ ID NO: 270 | DIVMTQSPKFLPVTAEDRVTITCKAS |
| mAb25-LFR2 | SEQ ID NO: 165 | VAWYQQKPGQSPKLLIY |
| mAb25-LFR3 | SEQ ID NO: 166 | NRYTGVPDRFTGSGSGTDFTFTISSVQV |
| | | EDLAVYFC |
| mAb25-LFR4 | SEQ ID NO: 174 | FGGGTKLEIK |
| mAb26-HFR1 | SEQ ID NO: 194 | EVQLQQSGTVLARPGASVKMSCKAS |
| mAb26-HFR2 | SEQ ID NO: 169 | MHWVKQRPGQGLEWIGA |
| mAb26-HFR3 | SEQ ID NO: 200 | SYNQKFKGKAKLTAVTSASTAYMELS |
| | | SLTNEDSAVYYC |
| mAb26-HFR4 | SEQ ID NO: 190 | WGQGTLVTVSA |
| mAb26-LFR1 | SEQ ID NO: 171 | DIVMTQSQKFMSTSVGDRVSVTCKAS |
| mAb26-LFR2 | SEQ ID NO: 240 | VAWYQKKPGQSPKALIY |
| mAb26-LFR3 | SEQ ID NO: 241 | TRYTGVPDRFTGSGSGTDFTLTISNVQS |
| | | EDLAEYFC |
| mAb26-LFR4 | SEQ ID NO: 174 | FGGGTKLEIK |
| mAb27-HFR1 | SEQ ID NO: 271 | EVQLQQSGPELVKPGASVKISCKAS |
| mAb27-HFR2 | SEQ ID NO: 169 | MHWVKQRPGQGLEWIGA |
| mAb27-HFR3 | SEQ ID NO: 272 | SYNQKFKDKAKLTAVTSANTAYMELS |
| | | SLTNEDSAVYYC |
| mAb27-HFR4 | SEQ ID NO: 190 | WGQGTLVTVSA |
| mAb27-LFR1 | SEQ ID NO: 171 | DIVMTQSQKFMSTSVGDRVSVTCKAS |
| mAb27-LFR2 | SEQ ID NO: 243 | VAWYQQKPGQSPKALIY |
| mAb27-LFR3 | SEQ ID NO: 273 | YRYSGVPDRFTGSGSGTDFTLTISSVQA |
| | | EDLAVYYC |
| mAb27-LFR4 | SEQ ID NO: 174 | FGGGTKLEIK |
| mAb28-HFR1 | SEQ ID NO: 274 | QVQLQQSGSELVKPGASVKLSCKAS |
| mAb28-HFR2 | SEQ ID NO: 275 | INWVKQRPGQGLEWIGW |
| mAb28-HFR3 | SEQ ID NO: 276 | KYNEKFKGKATLTVDTSSSTAYMELH |
| | | SLTSEDSAVYFC |
| mAb28-HFR4 | SEQ ID NO: 178 | WGQGTSVTVSS |
| mAb28-LFR1 | SEQ ID NO: 277 | DIELTQSPALMATSPGEKVTITCSVS |
| mAb28-LFR2 | SEQ ID NO: 278 | LHWYQQKSGTSPKLWIY |
| mAb28-LFR3 | SEQ ID NO: 279 | NLASGVPVRFSGSGSGTSYSLTISSMEA |
| | | EDAATYYC |
| mAb28-LFR4 | SEQ ID NO: 280 | FGAGTKLELK |
| mAb29-HFR1 | SEQ ID NO: 281 | QVQLQQSGTVLARPGASVKMSCKAS |
| mAb29-HFR2 | SEQ ID NO: 282 | MHWLKQRPGQGLEWIGA |
| mAb29-HFR3 | SEQ ID NO: 254 | NYNQNFKGKAKLTAVTSASTAYMELS |
| | | SLTNEDSAVYYC |
| mAb29-HFR4 | SEQ ID NO: 163 | WGQGTTLTVSS |
| mAb29-LFR1 | SEQ ID NO: 283 | DIVLTQSPASLAVSLGQKVTITCSVS |
| mAb29-LFR2 | SEQ ID NO: 278 | LHWYQQKSGTSPKLWIY |
| mAb29-LFR3 | SEQ ID NO: 279 | NLASGVPVRFSGSGSGTSYSLTISSMEA |
| | | EDAATYYC |
| mAb29-LFR4 | SEQ ID NO: 174 | FGGGTKLEIK |
| humAb1-VH | SEQ ID NO: 285 | QVQLVQSGAEVKKPGASVKVSCKASG |
| | | YTFTSYNMHWVRQAPGQGLEWMGSI |
| | | YPENGDTSYNQKFQDRVTLTRDTSTST |
| | | VYMELSSLRSEDTAVYYCARPFDYWG |
| | | QGTLVTVSS |
| humAb1-VL | SEQ ID NO: 286 | DIQMTQSPSSLSASVGDRVTITCQASQS |
| | | VGNNLAWYQQKPGKAPKLLIYYASNL |
| | | YTGVPSRFSGSGSGTDFTFTISSLQPEDI |
| | | ATYYCQQFYSSPYTFGQGTKLEIK |
| humAb2-VH | SEQ ID NO: 287 | QAQLVQSGAEVKKPGASVKVSCKASG |
| | | YTFTSYNMHWVRQAPGQGLEWMGSI |
| | | YPENGDTSYNQKFQDRVTLTRDTSTST |
| | | VYMELSSLRSEDTAVYYCARPFDYWG |
| | | QGTLVTVSS |
| humAb2-VL | SEQ ID NO: 288 | DIVMTQSPSSLSASVGDRVTITCQASQS |
| | | VGNNVAWYQQKPGKAPKLLIYYASNL |
| | | YTGVPSRFSGSGSGTDFTFTISSLQPEDI |
| | | ATYYCQQFYSSPYTFGQGTKLEIK |
| humAb3-VH | SEQ ID NO: 287 | QAQLVQSGAEVKKPGASVKVSCKASG |
| | | YTFTSYNMHWVRQAPGQGLEWMGSI |
| | | YPENGDTSYNQKFQDRVTLTRDTSTST |
| | | VYMELSSLRSEDTAVYYCARPFDYWG |
| | | QGTLVTVSS |
| humAb3-VL | SEQ ID NO: 289 | DIQMTQSPSSLSASVGDRVTITCQASQS |
| | | VGNNVAWYQQKPGKAPKLLIYYASNL |
| | | YTGVPSRFSGSGSGTDFTFTISSLQPEDI |
| | | ATYYCQQFYSSPYTFGQGTKLEIK |
| humAb4-VH | SEQ ID NO: 290 | QVQLVQSGAEVKKPGASVKVSCKASG |
| | | YTFTSYNMHWVRQAPGQGLEWMGSI |
| | | YPENGDTSYNQKFQGRVTLTRDTSTST |
| | | VYMELSSLRSEDTAVYYCARPFDYWG |
| | | QGTLVTVSS |
| humAb4-VL | SEQ ID NO: 288 | DIVMTQSPSSLSASVGDRVTITCQASQS |
| | | VGNNVAWYQQKPGKAPKLLIYYASNL |
| | | YTGVPSRFSGSGSGTDFTFTISSLQPEDI |
| | | ATYYCQQFYSSPYTFGQGTKLEIK |
| humAb5-VH | SEQ ID NO: 290 | QVQLVQSGAEVKKPGASVKVSCKASG |
| | | YTFTSYNMHWVRQAPGQGLEWMGSI |
| | | YPENGDTSYNQKFQGRVTLTRDTSTST |
| | | VYMELSSLRSEDTAVYYCARPFDYWG |
| | | QGTLVTVSS |
| humAb5-VL | SEQ ID NO: 286 | DIQMTQSPSSLSASVGDRVTITCQASQS |
| | | VGNNLAWYQQKPGKAPKLLIYYASNL |
| | | YTGVPSRFSGSGSGTDFTFTISSLQPEDI |
| | | ATYYCQQFYSSPYTFGQGTKLEIK |
| humAb6-VH | SEQ ID NO: 291 | QAQLVQSGAEVKKPGASVKVSCKASG |
| | | YTFTSYNMHWVRQAPGQGLEWMGSI |
| | | YPENGDTSYNQKFQGRVTLTRDTSTST |
| | | VYMELSSLRSEDTAVYYCARPFDYWG |
| | | QGTLVTVSS |
| humAb6-VL | SEQ ID NO: 286 | DIQMTQSPSSLSASVGDRVTITCQASQS |
| | | VGNNLAWYQQKPGKAPKLLIYYASNL |
| | | YTGVPSRFSGSGSGTDFTFTISSLQPEDI |
| | | ATYYCQQFYSSPYTFGQGTKLEIK |
| humAbA1-H | SEQ ID NO: 292 | QVQLVQSGAEVKKPGASVKVSCKASG |
| | | YTFTSYNMHWVRQAPGQGLEWMGSI |
| | | YPENGDTSYNQKFQDRVTLTRDTSTST |
| | | VYMELSSLRSEDTAVYYCARPFDYWG |
| | | QGTLVTVSSASTKGPSVFPLAPSSKSTS |
| | | GGTAALGCLVKDYFPEPVTVSWNSGA |
| | | LTSGVHTFPAVLQSSGLYSLSSVVTVPS |
| | | SSLGTQTYICNVNHKPSNTKVDKRVEP |
| | | KSCDKTHTCPPCPAPELLGGPSVFLFPP |
| | | KPKDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQVYT |
| | | LPPSREEMTKNQVSLTCLVKGFYPSDI |
| | | AVEWESNGQPENNYKTTPPVLDSDGS |
| | | FFLYSKLTVDKSRWQQGNVFSCSVMH |
| | | EALHNHYTQKSLSLSPG |
| humAbA1-L | SEQ ID NO: 293 | DIQMTQSPSSLSASVGDRVTITCQASQS |
| | | VGNNLAWYQQKPGKAPKLLIYYASNL |
| | | YTGVPSRFSGSGSGTDFTFTISSLQPEDI |
| | | ATYYCQQFYSSPYTFGQGTKLEIKRTV |
| | | AAPSVFIFPPSDEQLKSGTASVVCLLNN |
| | | FYPREAKVQWKVDNALQSGNSQESVT |
| | | EQDSKDSTYSLSSTLTLSKADYEKHKV |
| | | YACEVTHQGLSSPVTKSFNRGEC |
| humAbA2-H | SEQ ID NO: 294 | QVQLVQSGAEVKKPGASVKVSCKASG |
| | | YTFTSYNMHWVRQAPGQGLEWMGSI |
| | | YPENGDTSYNQKFQDRVTLTRDTSTST |
| | | VYMELSSLRSEDTAVYYCARPFDYWG |
| | | QGTLVTVSSASTKGPSVFPLAPSSKSTS |
| | | GGTAALGCLVKDYFPEPVTVSWNSGA |
| | | LTSGVHTFPAVLQSSGLYSLSSVVTVPS |
| | | SSLGTQTYICNVNHKPSNTKVDKRVEP |
| | | KSCDKTHTCPPCPAPELLGGPSVFLLPP |
| | | KPKDTLMISRTPEVTCVVVDVSHEDPE |
| | | VKFNWYVDGVEVHNAKTKPPEEQYN |
| | | STLRVVSILTVLHQDWLNGKEYKCKV |
| | | SNKALPAPIEKTISKAKGQPREPQVYTL |
| | | PPSREEMTKNQVSLTCLVKGFYPSDIA |
| | | VEWESNGQPENNYKTTPLVLDSDGSFF |
| | | LYSKLTVDKSRWQQGNVFSCSVMHEA |
| | | LHNHYTQKSLSLSPG |
| humAbA2-L | SEQ ID NO: 293 | DIQMTQSPSSLSASVGDRVTITCQASQS |
| | | VGNNLAWYQQKPGKAPKLLIYYASNL |
| | | YTGVPSRFSGSGSGTDFTFTISSLQPEDI |
| | | ATYYCQQFYSSPYTFGQGTKLEIKRTV |
| | | AAPSVFIFPPSDEQLKSGTASVVCLLNN |
| | | FYPREAKVQWKVDNALQSGNSQESVT |
| | | EQDSKDSTYSLSSTLTLSKADYEKHKV |
| | | YACEVTHQGLSSPVTKSFNRGEC |
| humAb1-HCDR1 | SEQ ID NO: 59 | GYTFTSYN |
| humAb1-HCDR2 | SEQ ID NO: 60 | IYPENGDT |
| humAb1-HCDR3 | SEQ ID NO: 61 | ARPFDY |
| humAb1-LCDR1 | SEQ ID NO: 62 | QSVGNN |
| humAb1-LCDR2 | SEQ ID NO: 63 | YAS |
| humAb1-LCDR3 | SEQ ID NO: 64 | QQFYSSPYT |
| humAb2-HCDR1 | SEQ ID NO: 59 | GYTFTSYN |
| humAb2-HCDR2 | SEQ ID NO: 60 | IYPENGDT |
| humAb2-HCDR3 | SEQ ID NO: 61 | ARPFDY |
| humAb2-LCDR1 | SEQ ID NO: 62 | QSVGNN |
| humAb2-LCDR2 | SEQ ID NO: 63 | YAS |
| humAb2-LCDR3 | SEQ ID NO: 64 | QQFYSSPYT |
| humAb3-HCDR1 | SEQ ID NO: 59 | GYTFTSYN |
| humAb3-HCDR2 | SEQ ID NO: 60 | IYPENGDT |
| humAb3-HCDR3 | SEQ ID NO: 61 | ARPFDY |
| humAb3-LCDR1 | SEQ ID NO: 62 | QSVGNN |
| humAb3-LCDR2 | SEQ ID NO: 63 | YAS |
| humAb3-LCDR3 | SEQ ID NO: 64 | QQFYSSPYT |
| humAb4-HCDR1 | SEQ ID NO: 59 | GYTFTSYN |
| humAb4-HCDR2 | SEQ ID NO: 60 | IYPENGDT |
| humAb4-HCDR3 | SEQ ID NO: 61 | ARPFDY |
| humAb4-LCDR1 | SEQ ID NO: 62 | QSVGNN |
| humAb4-LCDR2 | SEQ ID NO: 63 | YAS |
| humAb4-LCDR3 | SEQ ID NO: 64 | QQFYSSPYT |
| humAb5-HCDR1 | SEQ ID NO: 59 | GYTFTSYN |
| humAb5-HCDR2 | SEQ ID NO: 60 | IYPENGDT |
| humAb5-HCDR3 | SEQ ID NO: 61 | ARPFDY |
| humAb5-LCDR1 | SEQ ID NO: 62 | QSVGNN |
| humAb5-LCDR2 | SEQ ID NO: 63 | YAS |
| humAb5-LCDR3 | SEQ ID NO: 64 | QQFYSSPYT |
| humAb6-HCDR1 | SEQ ID NO: 59 | GYTFTSYN |
| humAb6-HCDR2 | SEQ ID NO: 60 | IYPENGDT |
| humAb6-HCDR3 | SEQ ID NO: 61 | ARPFDY |
| humAb6-LCDR1 | SEQ ID NO: 62 | QSVGNN |
| humAb6-LCDR2 | SEQ ID NO: 63 | YAS |
| humAb6-LCDR3 | SEQ ID NO: 64 | QQFYSSPYT |
| humAbA1-HCDR1 | SEQ ID NO: 59 | GYTFTSYN |
| humAbA1-HCDR2 | SEQ ID NO: 60 | IYPENGDT |
| humAbA1-HCDR3 | SEQ ID NO: 61 | ARPFDY |
| humAbA1-LCDR1 | SEQ ID NO: 62 | QSVGNN |
| humAbA1-LCDR2 | SEQ ID NO: 63 | YAS |
| humAbA1-LCDR3 | SEQ ID NO: 64 | QQFYSSPYT |
| humAbA2-HCDR1 | SEQ ID NO: 59 | GYTFTSYN |
| humAbA2-HCDR2 | SEQ ID NO: 60 | IYPENGDT |
| humAbA2-HCDR3 | SEQ ID NO: 61 | ARPFDY |
| humAbA2-LCDR1 | SEQ ID NO: 62 | QSVGNN |
| humAbA2-LCDR2 | SEQ ID NO: 63 | YAS |
| humAbA2-LCDR3 | SEQ ID NO: 64 | QQFYSSPYT |
| humAb1-HFR1 | SEQ ID NO: 305 | QVQLVQSGAEVKKPGASVKVSCKAS |
| humAb1-HFR2 | SEQ ID NO: 296 | MHWVRQAPGQGLEWMGS |
| humAb1-HFR3 | SEQ ID NO: 297 | SYNQKFQDRVTLTRDTSTSTVYMELSS |
| | | LRSEDTAVYYC |
| humAb1-HFR4 | SEQ ID NO: 298 | WGQGTLVTVSSASTKGPSVFP |
| humAb1-LFR1 | SEQ ID NO: 299 | DIQMTQSPSSLSASVGDRVTITCQAS |
| humAb1-LFR2 | SEQ ID NO: 300 | LAWYQQKPGKAPKLLIY |
| humAb1-LFR3 | SEQ ID NO: 301 | NLYTGVPSRFSGSGSGTDFTFTISSLQP |
| | | EDIATYYC |
| humAb1-LFR4 | SEQ ID NO: 302 | FGQGTKLEIKRTVAAPSVFIFP |
| humAb2-HFR1 | SEQ ID NO: 295 | QAQLVQSGAEVKKPGASVKVSCKAS |
| humAb2-HFR2 | SEQ ID NO: 296 | MHWVRQAPGQGLEWMGS |
| humAb2-HFR3 | SEQ ID NO: 297 | SYNQKFQDRVTLTRDTSTSTVYMELSS |
| | | LRSEDTAVYYC |
| humAb2-HFR4 | SEQ ID NO: 298 | WGQGTLVTVSSASTKGPSVFP |
| humAb2-LFR1 | SEQ ID NO: 303 | DIVMTQSPSSLSASVGDRVTITCQAS |
| humAb2-LFR2 | SEQ ID NO: 304 | VAWYQQKPGKAPKLLIY |
| humAb2-LFR3 | SEQ ID NO: 301 | NLYTGVPSRFSGSGSGTDFTFTISSLQP |
| | | EDIATYYC |
| humAb2-LFR4 | SEQ ID NO: 302 | FGQGTKLEIKRTVAAPSVFIFP |
| humAb3-HFR1 | SEQ ID NO: 295 | QAQLVQSGAEVKKPGASVKVSCKAS |
| humAb3-HFR2 | SEQ ID NO: 296 | MHWVRQAPGQGLEWMGS |
| humAb3-HFR3 | SEQ ID NO: 297 | SYNQKFQDRVTLTRDTSTSTVYMELSS |
| | | LRSEDTAVYYC |
| humAb3-HFR4 | SEQ ID NO: 298 | WGQGTLVTVSSASTKGPSVFP |
| humAb3-LFR1 | SEQ ID NO: 299 | DIQMTQSPSSLSASVGDRVTITCQAS |
| humAb3-LFR2 | SEQ ID NO: 304 | VAWYQQKPGKAPKLLIY |
| humAb3-LFR3 | SEQ ID NO: 301 | NLYTGVPSRFSGSGSGTDFTFTISSLQP |
| | | EDIATYYC |
| humAb3-LFR4 | SEQ ID NO: 302 | FGQGTKLEIKRTVAAPSVFIFP |
| humAb4-HFR1 | SEQ ID NO: 305 | QVQLVQSGAEVKKPGASVKVSCKAS |
| humAb4-HFR2 | SEQ ID NO: 296 | MHWVRQAPGQGLEWMGS |
| humAb4-HFR3 | SEQ ID NO: 306 | SYNQKFQGRVTLTRDTSTSTVYMELSS |
| | | LRSEDTAVYYC |
| humAb4-HFR4 | SEQ ID NO: 298 | WGQGTLVTVSSASTKGPSVFP |
| humAb4-LFR1 | SEQ ID NO: 303 | DIVMTQSPSSLSASVGDRVTITCQAS |
| humAb4-LFR2 | SEQ ID NO: 304 | VAWYQQKPGKAPKLLIY |
| humAb4-LFR3 | SEQ ID NO: 301 | NLYTGVPSRFSGSGSGTDFTFTISSLQP |
| | | EDIATYYC |
| humAb4-LFR4 | SEQ ID NO: 302 | FGQGTKLEIKRTVAAPSVFIFP |
| humAb5-HFR1 | SEQ ID NO: 305 | QVQLVQSGAEVKKPGASVKVSCKAS |
| humAb5-HFR2 | SEQ ID NO: 296 | MHWVRQAPGQGLEWMGS |
| humAb5-HFR3 | SEQ ID NO: 306 | SYNQKFQGRVTLTRDTSTSTVYMELSS |
| | | LRSEDTAVYYC |
| humAb5-HFR4 | SEQ ID NO: 298 | WGQGTLVTVSSASTKGPSVFP |
| humAb5-LFR1 | SEQ ID NO: 299 | DIQMTQSPSSLSASVGDRVTITCQAS |
| humAb5-LFR2 | SEQ ID NO: 300 | LAWYQQKPGKAPKLLIY |
| humAb5-LFR3 | SEQ ID NO: 301 | NLYTGVPSRFSGSGSGTDFTFTISSLQP |
| | | EDIATYYC |
| humAb5-LFR4 | SEQ ID NO: 302 | FGQGTKLEIKRTVAAPSVFIFP |
| humAb6-HFR1 | SEQ ID NO: 295 | QAQLVQSGAEVKKPGASVKVSCKAS |
| humAb6-HFR2 | SEQ ID NO: 296 | MHWVRQAPGQGLEWMGS |
| humAb6-HFR3 | SEQ ID NO: 306 | SYNQKFQGRVTLTRDTSTSTVYMELSS |
| | | LRSEDTAVYYC |
| humAb6-HFR4 | SEQ ID NO: 298 | WGQGTLVTVSSASTKGPSVFP |
| humAb6-LFR1 | SEQ ID NO: 299 | DIQMTQSPSSLSASVGDRVTITCQAS |
| humAb6-LFR2 | SEQ ID NO: 300 | LAWYQQKPGKAPKLLIY |
| humAb6-LFR3 | SEQ ID NO: 301 | NLYTGVPSRFSGSGSGTDFTFTISSLQP |
| | | EDIATYYC |
| humAb6-LFR4 | SEQ ID NO: 302 | FGQGTKLEIKRTVAAPSVFIFP |
| humAbA1-HFR1 | SEQ ID NO: 305 | QVQLVQSGAEVKKPGASVKVSCKAS |
| humAbA1-HFR2 | SEQ ID NO: 296 | MHWVRQAPGQGLEWMGS |
| humAbA1-HFR3 | SEQ ID NO: 297 | SYNQKFQDRVTLTRDTSTSTVYMELSS |
| | | LRSEDTAVYYC |
| humAbA1-HFR4 | SEQ ID NO: 298 | WGQGTLVTVSSASTKGPSVFP |
| humAbA1-LFR1 | SEQ ID NO: 299 | DIQMTQSPSSLSASVGDRVTITCQAS |
| humAbA1-LFR2 | SEQ ID NO: 300 | LAWYQQKPGKAPKLLIY |
| humAbA1-LFR3 | SEQ ID NO: 301 | NLYTGVPSRFSGSGSGTDFTFTISSLQP |
| | | EDIATYYC |
| humAbA1-LFR4 | SEQ ID NO: 302 | FGQGTKLEIKRTVAAPSVFIFP |
| humAbA2-HFR1 | SEQ ID NO: 305 | QVQLVQSGAEVKKPGASVKVSCKAS |
| humAbA2-HFR2 | SEQ ID NO: 296 | MHWVRQAPGQGLEWMGS |
| humAbA2-HFR3 | SEQ ID NO: 297 | SYNQKFQDRVTLTRDTSTSTVYMELSS |
| | | LRSEDTAVYYC |
| humAbA2-HFR4 | SEQ ID NO: 298 | WGQGTLVTVSSASTKGPSVFP |
| humAbA2-LFR1 | SEQ ID NO: 299 | DIQMTQSPSSLSASVGDRVTITCQAS |
| humAbA2-LFR2 | SEQ ID NO: 300 | LAWYQQKPGKAPKLLIY |
| humAbA2-LFR3 | SEQ ID NO: 301 | NLYTGVPSRFSGSGSGTDFTFTISSLQP |
| | | EDIATYYC |
| humAbA2-LFR4 | SEQ ID NO: 302 | FGQGTKLEIKRTVAAPSVFIFP |
| HumAbA1 CH1 | SEQ ID NO: 307 | ASTKGPSVFPLAPSSKSTSGGTAALGCL |
| | | VKDYFPEPVTVSWNSGALTSGVHTFPA |
| | | VLQSSGLYSLSSVVTVPSSSLGTQTYIC |
| | | NVNHKPSNTKVDKRV |
| HumAbA1 Hinge | SEQ ID NO: 308 | EPKSCDKTHTCPPCP |
| HumAbA1 CH2 | SEQ ID NO: 309 | APELLGGPSVFLFPPKPKDTLMISRTPE |
| | | VTCVVVDVSHEDPEVKFNWYVDGVE |
| | | VHNAKTKPREEQYNSTYRVVSVLTVL |
| | | HQDWLNGKEYKCKVSNKALPAPIEKTI |
| | | SKAK |
| HumAbA1 CH3 | SEQ ID NO: 310 | GQPREPQVYTLPPSREEMTKNQVSLTC |
| | | LVKGFYPSDIAVEWESNGQPENNYKTT |
| | | PPVLDSDGSFFLYSKLTVDKSRWQQG |
| | | NVFSCSVMHEALHNHYTQKSLSLSPG |
| HumAbA1 CL | SEQ ID NO: 311 | RTVAAPSVFIFPPSDEQLKSGTASVVCL |
| | | LNNFYPREAKVQWKVDNALQSGNSQE |
| | | SVTEQDSKDSTYSLSSTLTLSKADYEK |
| | | HKVYACEVTHQGLSSPVTKSFNRGEC |
| HumAbA2 CH1 | SEQ ID NO: 307 | ASTKGPSVFPLAPSSKSTSGGTAALGCL |
| | | VKDYFPEPVTVSWNSGALTSGVHTFPA |
| | | VLQSSGLYSLSSVVTVPSSSLGTQTYIC |
| | | NVNHKPSNTKVDKRV |
| HumAbA2 Hinge | SEQ ID NO: 308 | EPKSCDKTHTCPPCP |
| HumAbA2 CH2 | SEQ ID NO: 312 | APELLGGPSVFLLPPKPKDTLMISRTPE |
| | | VTCVVVDVSHEDPEVKFNWYVDGVE |
| | | VHNAKTKPPEEQYNSTLRVVSILTVLH |
| | | QDWLNGKEYKCKVSNKALPAPIEKTIS |
| | | KAK |
| HumAbA2 CH3 | SEQ ID NO: 313 | GQPREPQVYTLPPSREEMTKNQVSLTC |
| | | LVKGFYPSDIAVEWESNGQPENNYKTT |
| | | PLVLDSDGSFFLYSKLTVDKSRWQQG |
| | | NVFSCSVMHEALHNHYTQKSLSLSPG |
| HumAbA2 CL | SEQ ID NO: 311 | RTVAAPSVFIFPPSDEQLKSGTASVVCL |
| | | LNNFYPREAKVQWKVDNALQSGNSQE |
| | | SVTEQDSKDSTYSLSSTLTLSKADYEK |
| | | HKVYACEVTHQGLSSPVTKSFNRGEC |

### Specific Binding

The anti-FGFR2b antibodies provided herein are capable of specifically binding to FGFR2b.

In some embodiments, the antibody described herein specifically binds fibroblast growth factor receptor 2b (FGFR2b).

In some embodiments, the antibody described herein binds FGFR2b with a K_{D} of about 10 nM or lower. In some embodiments, the antibody described herein binds FGFR2b with a K_{D} of about 9 nM or lower. In some embodiments, the antibody described herein binds FGFR2b with a K_{D} of about 5 nM or lower. In some embodiments, the antibody described herein binds FGFR2b with a K_{D} of about 4 nM, 3 nM, 2 nM, 1 nM, 0.1 nM or lower.

In some embodiments, the FGFR2b is a human FGFR2b. In some embodiments, the FGFR2b is a mouse FGFR2b or a cynomolgus monkey FGFR2b.

In certain embodiments, the antibodies provided herein have a specific binding affinity to human FGFR2b which is sufficient to provide for diagnostic and/or therapeutic use. For example, the antibody described herein can be used to detect existence of FGFR2 in a sample, by contacting the antibody described herein or a composition thereof with the sample, wherein a detection of at least one bound antibody indicates presence of FGFR2b. By quantifying the bound antibody, this method can also be used to detect overexpression of FGFR2 gene, which is closely related to aberrant FGFR2 signaling and cancer.

In some embodiments, the antibody described herein can be used to block FGFR2b, in particular human FGFR2b, from binding with its ligand. Thus, the antibody described herein can provide biological activity including, for example, inhibition of the proliferation of FGFR2b overexpressing cells.

In some embodiments, the antibody described herein does not bind to FGFR1b, FGFR1c, FGFR2c, FGFR3b, FGFR3c, or FGFR4. In some embodiments, the antibody described herein does not detectably bind to FGFR1b, FGFR1c, FGFR2c, FGFR3b, FGFR3c, or FGFR4. As used herein, the phrase “detectable binding” or “detectably bind” means that the K_{D} value measured by Bio-layer interferometry (BLI) using Octet® BLI Label-Free Detection Systems is no lower than 0.1, or the OD450 value measured by ELISA at 50 nM is no lower than 1.0. The protocols for BLI and ELISA are described in Example 3 below.

The antibodies described herein demonstrate sub-nanomolar equilibrium dissociation constants (K_{D}) for mouse, cynomolgus monkey, and human FGFR2b extra-cellular domain (ECD) with comparable associate and dissociate constants (Kon and K_{off}). The antibodies described herein have good cross-species affinity. Thus, mouse and cynomolgus monkey are deemed appropriate species in which to perform toxicology studies with the antibodies described herein.

### Compositions

In another aspect, the present disclosure provides a composition comprising the antibody described herein.

In another aspect, the present disclosure provides a pharmaceutical composition, comprising the antibody described herein and a pharmaceutically acceptable carrier.

The composition or the pharmaceutical composition described herein may comprise a pharmaceutically acceptable carrier, diluent, or excipient. As used herein “pharmaceutically acceptable carrier, diluent, or excipient” includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose, and sucrose; starches, such as corn starch and potato starch; cellulose, and its derivatives, such as sodium carboxymethyl cellulose, ethyl cellulose, and cellulose acetate; tragacanth; malt; gelatin; talc; cocoa butter; waxes; animal and vegetable fats; paraffins; silicones; bentonites; silicic acid; zinc oxide; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate, and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

The liquid pharmaceutical compositions, whether they be solutions, suspensions or other like form, may include one or more of the following: sterile diluents such as water for injection, saline solution, preferably physiological saline; Ringers solution; isotonic sodium chloride; fixed oils such as synthetic mono or diglycerides which may serve as the solvent or suspending medium; polyethylene glycols; glycerin; propylene glycol or other solvents; antibacterial agents, such as benzyl alcohol or methyl paraben; antioxidants such as ascorbic acid or sodium bisulfite; chelating agents, such as ethylenediaminetetraacetic acid; buffers such as acetates, citrates, or phosphates; and agents for the adjustment of tonicity, such as sodium chloride or dextrose. The parenteral preparation can be enclosed in ampoules, disposable syringes, or multiple dose vials made of glass or plastic. An injectable pharmaceutical composition is preferably sterile.

The composition may be suitably developed for intravenous, intratumoral, oral, rectal, vaginal, parenteral, topical, pulmonary, intranasal, buccal, ophthalmic, or another route of administration.

### Polynucleotides

In another aspect, the present disclosure provides a polynucleotide encoding the antibody described herein.

The polynucleotide sequences encoding the antibody described herein can be operably linked to one or more regulatory elements, such as a promoter and enhancer, that allow expression of the nucleotide sequence in the intended host cell. The polynucleotide may be a DNA or an RNA, such as a cDNA or a mRNA. The polynucleotide described herein is obtained by methods readily available in the arts.

### Vectors

In another aspect, the present disclosure provides a vector, comprising the polynucleotide described herein. Such vectors may be plasmid vectors, viral vectors, vectors for baculovirus expression, transposon-based vectors, or any other vector suitable for introduction of the polynucleotide of the disclosure into a given organism or genetic background by any means. For example, polynucleotides encoding the antibody described herein may be inserted into expression vectors. The DNA segments encoding the antibody may be operably linked to control sequences in the expression vector(s) that ensure the expression of immunoglobulin polypeptides. Such control sequences include signal sequences, promoters (e.g., naturally associated or heterologous promoters), enhancer elements, and transcription termination sequences, and are chosen to be compatible with the host cell chosen to express the antibody. Once the vector has been incorporated into the appropriate host, the host is maintained under conditions suitable for high level expression of the proteins encoded by the incorporated polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance, or neomycin resistance to permit detection of those cells transformed with the desired DNA sequences. Suitable vectors, promoter, and enhancer elements are known in the art; many are commercially available for generating subject recombinant constructs.

The term “host cell” refers to a cell into which a vector has been introduced. It is understood that the term host cell is intended to refer not only to the particular subject cell but to the progeny of such a cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells, or archaeal cells. Escherichia coli, bacilli, such as Bacillus subtilis, and other Enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian, or other animal origins.

### Cells

In another aspect, the present disclosure provides a cell capable of expressing the antibody described herein.

In another aspect, the present disclosure provides a cell comprising the polynucleotide described herein and/or the vector described herein.

The cell described herein include, but not limited to, eukaryotic cells, prokaryotic cells, plant cells, or archaeal cells. Exemplary eukaryotic cells include, but are not limited to, mammalian, insect, avian, or other animal origins.

Exemplary prokaryotic cells include, but are not limited to, Escherichia coli, bacilli, such as Bacillus subtilis, and other Enterobacteriaceae, such as Salmonella, Serratia, and various Pseudomonas species. Other microbes, such as yeast, are also useful for expression. Saccharomyces (e.g., S. cerevisiae) and Pichia are examples of suitable yeast host cells.

The cell as described herein includes various cells derived from individual animals, subcultured cells, primary cultured cells, cell lines, recombinant cells, and microbial cells.

In some embodiments, the cell is a hybridoma cell. In some embodiments, the hybridoma cell is obtained by fusing myeloma cells with cells producing the antibody described herein. In some embodiments, the cell producing the antibody described herein is a splenocyte.

### Methods

In another aspect, the present disclosure provides a method of producing an antibody, comprising culturing the cell described herein and recovering the antibody from the cell.

The antibody described herein can be obtained by any method known in the art. For example, monoclonal antibodies can be obtained through the following steps: (1) preparing antigen; (2) immunization; (3) preparing spleen cells; (4) preparing myeloma cells; (5) fusing the spleen cells with myeloma cells; (6) screening for hybridomas producing antibodies; and (7) obtaining single cell clones

Here, an exemplary method for generating a monoclonal antibody described herein is provided. First, animals, such as rats or mice, are immunized with the recombinant human FGFR2b-FC protein or its soluble form according to the method of Kohler and Milstein (Köhler et al., Nature, 1975; 256 (5517), 495-497; Kennet, R. H., T. J. Mckeam, and K. B. Bechtol. “Methods for production and characterization of monoclonal antibodies.” Monoclonal Antibodies (Appendix), R H Kennet, T J Mckeam, and K B Bechtol, eds., Plenum, New York, 1980). The recombinant human FGFR2b antigen are available from commercial distributors. Then cells producing the antibody described herein are isolated from the spleens of the immunized animals. These cells are then fused with myeloma cells to thereby establish hybridomas. Monoclonal antibodies as described herein can be obtained from cultures of these hybridomas.

The strain of mice or rats is not limited. In the case of mice, for example, BALB/c, C57BL, CD1, SJL can be used. These mice are available from laboratory animal breeders or distributors. The BALB/c mouse strain is preferred. The mouse is preferably 5 to 12 weeks old, more preferably 6 to 8 weeks old, at the time of immunization.

Exemplary methods for determining antibody titers include, but are not limited to, immunoassay such as ELISA.

The antibody-producing cells derived from spleen cells or lymphocytes separated from immunized animals, can be prepared according to a method known in the art. The myeloma cells used in cell fusion are not particularly limited and can be selected appropriately for use for cell lines know in the art. The antibodies-producing cells can be fused with the myeloma cells under conditions that prevent cell viability from being exceeding reduced, according to any method known in the art.

The screening of hybridomas includes, but are not limited to, protein-level binding and blocking, cellular binding and blocking, signaling pathway blocking assay, and cell proliferation inhibit assay. The hybridoma can be cloned using the limiting dilution method.

The antibodies described herein can also be produced by recombinant expression. Desired antibody can be expressed in any organism suitable to produce the required amounts and forms of the antibody. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modification that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Expression vectors can be introduced into host cells via, for example, transformation, transfection, transduction, infection, electroporation, and sonoporation. A skilled artisan is able to select methods and conditions suitable for introducing an expression vector into host cells.

Following the introduction of a vector comprising a selectable marker, cells can be allowed to grow for 1-2 days in an enriched media before they are switched to selective media. The purpose of the selectable marker is to confer resistance to selection, and its presence allows growth and recovery of cells that successfully express the introduced sequences. Resistant cells of stably transformed cells can be proliferated using tissue culture techniques appropriate to the cell types. In some embodiments, the antibody described herein is expressed in a mammalian expression system. Expression constructs can be transferred to mammalian cells by viral infection, such as by adenovirus constructs, or by direct DNA transfer, such as liposomes, calcium phosphate, DEAE-dextran, and by physical means such as electroporation and microinjection. In some embodiments, the antibody described herein is delivered using viral transduction, for example, with a vector.

Once the vector has been incorporated into the appropriate host cell, the host cell is maintained under conditions suitable for expression of the antibody encoded by the incorporated polynucleotides. A skilled artisan is able to select conditions suitable for expression of the antibody described herein.

The antibodies described herein can be purified according to any known methods in the art, such as protein A chromatography, anion exchange chromatography, and cation exchange chromatography. It's understood that a person skilled in the art is capable of selecting suitable methods and conditions for purifying the antibodies described herein.

The present disclosure provides methods to inhibit growth of FGFR2b-positive cancer cells and the progression of neoplastic disease by providing novel anti-FGFR2b antibodies. The anti-FGFR2b antibodies of the present disclosure that binds FGFR2IIIb but not detectably to FGFR2IIIc, could partially or completely inhibit one or more biological activities of FGFR2b. The First important activity of FGFR2b that can be inhibited by the anti-FGFR2b antibodies described herein is the ability of FGFR2b to bind to one or more or all of its FGF ligands, for example, but are not limited to, FGF1, FGF3, FGF7, FGF10 and FGF22. The second activity of FGFR2b that can be inhibited by the anti-FGFR2b antibodies described herein is stimulation of proliferation of cells e.g., cancer cells that overexpressed FGFR2b. Other functions of FGFR2b which can be inhibited by the anti-FGFR2b antibodies described herein include, but are not limited to, the signal transduction pathway stimulated by binding of FGFs ligand to FGFR2b, e.g., phosphorylation of FGFR2b. In some embodiments, the anti-FGFR2b antibodies described herein inhibit these activities that are induced by one or more of the FGFs listed above.

The anti-FGFR2b antibodies described herein can also inhibit the growth of a tumor Xenograft that overexpresses FGFR2b, e.g., a SNU-16 or OCUM-2M xenograft.

In another aspect, the present disclosure provides a method for blocking FGFR2b from binding to at least one fibroblast growth factor (FGF), comprising administering to a subject an effective amount of the antibody described herein, or the composition described herein. In some embodiments, the FGF is selected from FGF1, FGF3, FGF7, FGF10, and FGF22. Fibroblast growth factors (FGF) are a family of cell signalling proteins produced by macrophages; they are involved in a wide variety of processes, most notably as crucial elements for normal development in animal cells. Any irregularities in their function lead to a range of developmental defects. These growth factors typically act as systemic or locally circulating molecules of extracellular origin that activate cell surface receptors.

In another aspect, the present disclosure provides a method for inhibiting cell proliferation, comprising administering to a subject an effective amount of the antibody described herein, or the composition described herein. In some embodiments, the cell proliferation is FGF-induced cell proliferation. In some embodiments, the cell is a cancer cell.

In another aspect, the present disclosure provides a method for inhibiting the signal transduction pathway stimulated by binding of FGF to FGFR2b, comprising administering to a subject an effective amount of the antibody described herein, or the composition described herein.

In another aspect, the present disclosure provides a method for inhibiting the growth of a tumor xenograft that overexpresses FGFR2b, comprising administering to a subject an effective amount of the antibody described herein, or the composition described herein.

In another aspect, the present disclosure provides a method for detecting the presence of FGFR2b in a sample, comprising contacting the antibody described herein, or the composition described herein with the sample, wherein a detection of at least one bound antibody indicates presence of FGFR2b.

### EXAMPLES

The present disclosure may be further described by the following non-limiting examples, in which standard techniques known to the skilled artisan and techniques analogous to those described in these examples may be used where appropriate. It is understood that the skilled artisan will envision additional embodiments consistent with the disclosure provided herein.

The examples discussed below are intended to be purely exemplary of the invention and should not be considered to limit the invention in any way. The examples are not intended to represent that the experiments below are all or the only experiments performed.

### Example 1 Cells and Reagents

Human gastric cancer cell line KATO III and tool cell line HEK 293 were purchased from Chinese Academy of Sciences. Human gastric cancer cell line SNU16 was purchased from BeNa Culture Collection (BNCC). The above-described human cell lines were cultured according to the suppliers' recommendations.

To establish cell-based assays for antibody screening during antibody generation period, HEK293 cells were engineered to express FGFR2b or FGFR2c. The HEK293 cells were transfected with plasmids encoding FGFR2b or FGFR2c isoforms of human FGFR2. Following selection with puromycin, single clone with high expression of FGFR2b or FGFR2c was isolated.

Human FGFR2 alpha (IIIb)-Fc (Cat. 16485-H02H) and Human FGFR1alpha (IIIb)-His (Cat. 16482-H08H) were purchased from Sino Biological China. Human FGFR1/CD331-His (Cat. FG1-H5223) and Human FGFR4/CD334-His (Cat. FG4-H5228) were purchased from ACRO Biosystems China. Human FGFR2 alpha (IIIb)-His (Cat. FGR-HM1BD), human FGFR2 alpha (IIIc)-His (Cat. FGR-HM2CD), Human FGFR3 alpha (IIIb)-His (Cat. FGF-HM43B) and human FGFR3 alpha (IIIc)-His (Cat. FGF-HM43C) were purchased from Kactus Biosystems China. Mouse FGFR2 (canonical)-Fc (Cat. 51128-M02H) was purchased from Sino Biological China. Cynomolgus FGFR2 (beta IIIb)-His (Cat. FGF-CM1BB) was purchased from Kactus Biosystems China. KGF/FGF-7 Protein, Human, Recombinant (His Tag) (Cat. 10210-H07E) was purchased from Sino Biological China. Human FGF10 Protein (Cat. FGF-HE010) was purchased from Kactus Biosystems China.

A clinical stage anti-human FGFR2b specific antibody FPA144 was expressed according to the related patent application WO 2015/017600 A1.

### Example 2. Generation of Anti-FGFRs Monoclonal Ab

BALB/c mice, CD1 mice or SJL mice were immunized with human FGFR2b (alpha)-Fc in SAS/CpG S.C. at an initial dose of 50 μg/mouse, and 25 μg/mouse 3 weeks later, and 25 ug/mouse 3 weeks later. The serum titer against human FGFR2b-Fc was determined by ELISA. Four days after the final injection, spleen cells were extracted and fused with mouse myeloma cells. Seven days after the fusion, hybridoma culture supernatants were screened first for FGFR2b (beta)-His binding by the ELISA. Hybridomas pass primary screening were subjected to secondary screening panel, including binding to KATO III cells by FACS. Hybridomas pass second screening were selected and subcloned. The monoclonal mouse antibody obtained is mAb1.

The heavy and light chain variable (VH, VL) regions sequences of the mAb1 was determined using standard RACE technology. Total RNA was extracted from the selected hybridoma cell line. Then full-length first strand cDNA containing 5′ ends were generated using SMART RACE cDNA Amplification Kit (Clontech) according to manufacturer's instructions, and amplified by PCR. PCR products were isolated and purified, and then TA cloned and sequenced.

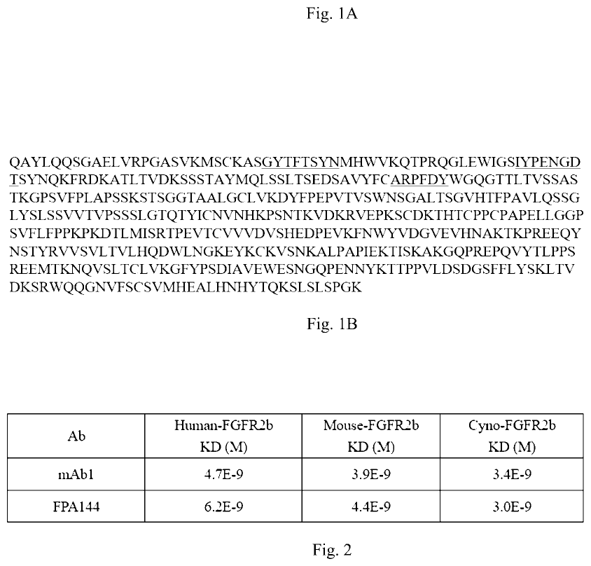

Then chimeric antibody mAb1 was generated by grafting the VH and VL of mouse Ab 1 into a human Fc. Amino acid sequences of the entire mAb1 light chain and heavy chain are shown in FIG. 1.

### Example 3. Binding Characteristics of the Antibodies

The binding of antibodies to human FGFR2b antigen was determined by Biolayer Interferometry (BLI) (OCTET). Briefly, Protein A Biosensor was firstly prewet by a 10 min injection of PBST (0.05% Tween 20). Antibodies were diluted in PBST-BSA (0.05% Tween, 0.1% BSA) and injected at serial concentration (0, 1.56, 3.12, 6.25, 12.5, 25, 50 nM) and surface regeneration of the Protein A Biosensor were included in each running cycle. The association constant, dissociation constant was calculated with OCTET RED384 evaluation software (version 1.0). As shown in FIG. 2, mAb1 exhibited strong binding affinity (nanomolar) to human, mouse and cynomolgus monkey FGFR2b, and is better than or similar to competitor antibody FPA144.

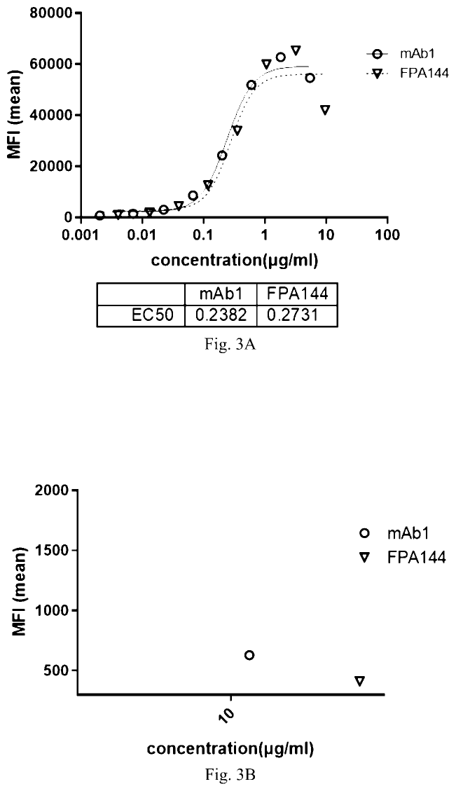

To confirm that the selected antibody can bind to the endogenous forms of FGFR2b on the cell membrane, flow cytometry was performed using human HEK293 cells which transfected with genes for FGFR2IIIc and FGFR2IIIb. FGFR2IIIb-HEK293 or FGFR2IIIc-HEK293 monoclone were grown in DMEM medium with 10% FBS and then seed in 100,000/well and wash with PBS+0.5% FBS. Then added various concentration mAbs for 30 min, followed by incubation with fluorescent coupled anti human IgG Fc secondary antibody for 30 min. The fluorescence signal was detected by Cytoflex. This binding activity data of mAb1 was processed using Graphpad Prism and the data was shown in FIG. 3. As seen in Figure. 3B, mAb1 bind to the FGFR2IIIb-transfected cells, as expected from their epitopes, and cannot bind to FGFR2IIIc transfected cells.

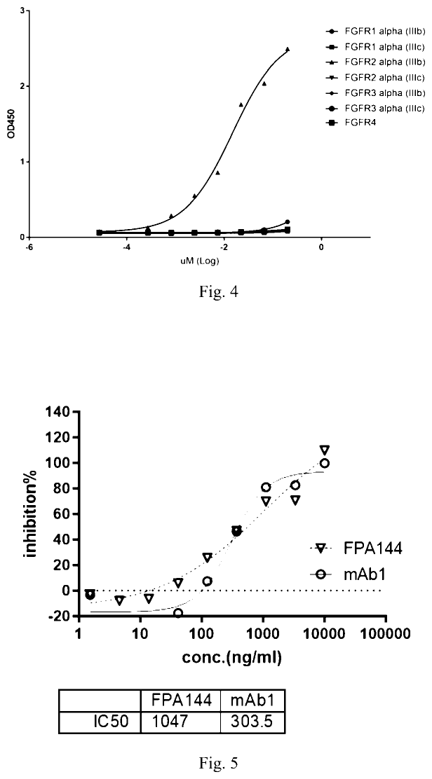

The binding specificity of mAb1 with various FGFR family member, FGFR1b, FGFR1c, FGFR2b, FGFR2c, FGFR3b, FGFR3c and FGFR4 was characterized with ELISA assay. In brief, 96-well ELISA plate were coated with about 50 ng/well 0.5 mg/ml human FGFR1 alpha (IIIb)-His, human FGFR1/CD331 protein-His, human FGFR2 alpha (IIIb) Protein, human FGFR2 alpha (IIIc) Protein, human FGFR3 alpha (IIIb)-His, human FGFR3 alpha (IIIc)-His, and human FGFR4/CD334-His in PH 9.6 carbonate buffer solution overnight. Then the plate was blocked with 2% BSA in PBST (PBS with 0.05% Tween20) and incubation with antibody samples for 60 min at room temperature, and then washed once in PBST and followed by incubation with mAb1 (0.2 μm first hole, 3 times down 7 steps, the last step down 10 times) at room temperature. Then washed once in PBST and followed by incubation with Peroxidase-conjugated AffiniPure F (AB′) 2 Fragment Goat Anti-Human IgG (Jackson Immuno Research, #109-036-098) conjugate for 30 min HRP activity was detected with TMB Substrate (Solarbio, #PR1200) and the reaction was stopped with stop solution (Solarbio, #C1058). The plate was read at 450 nm. The data is shown in FIG. 4. According to the result of ELISA analysis, mAb1 specifically binds to FGFR2b, and it does not bind to any other FGFR family members.

### Example 4. In vitro inhibition activity

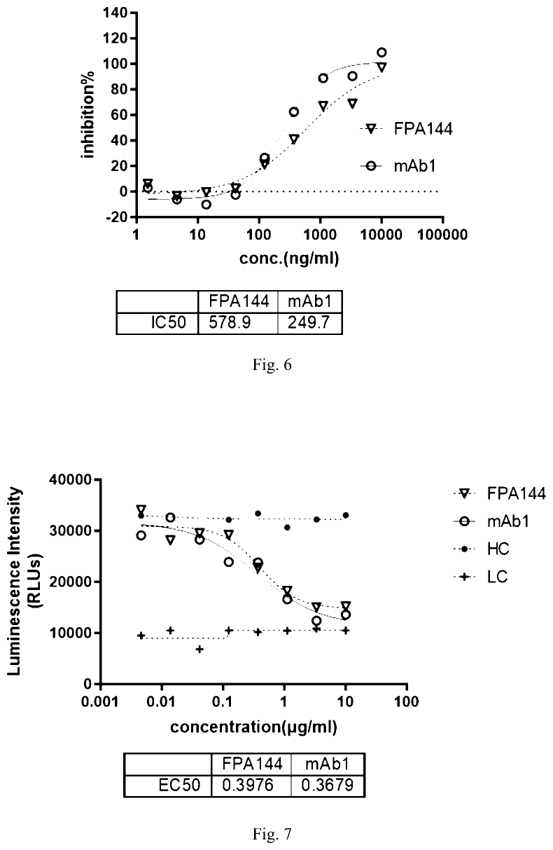

The inhibition activity of antibody on ligand-induced cell proliferation was done in SNU 16 cell clones. Cells were seeded in 96-well plates at 5,000 cell/well in RPMI1640 medium containing 0.5% fetal bovine serum. Starve the cell for 16 hours/overnight. SNU-16 cells were treated with varying concentrations of antibodies diluted in RPMI media with 0.5% FBS for 30 minutes. SNU-16 cells were then treated with 100 ng/mL FGF7 or FGF10 with 1 μg/mL heparin (final concentrations) diluted in RPMI media with 0% FBS, and incubated at 37° C. with 5% CO₂ for 4 days. Add 50 μL CTG, read luminescence as T144H. This inhibition activity data of mAb1 was processed using Graphpad Prism and the data was shown in FIG. 5 and FIG. 6. In the presence of increasing concentrations of mAb1, FGF7-induced (FIG. 5) and FGF10-induced (FIG. 6) cell proliferation was inhibited in a concentration-dependent manner in SNU-16 cells, the potency of mAB1 was better than FPA144.

The inhibition of FGFR2 signaling pathway by the antibody was investigated. Anti FGFR2b antibody against human FGFR2IIIb by SRE reporter assay. HEK293 cell lines stably expressed the luciferase reporter driven by serum response element (SRE) and human fibroblast growth factor receptor (FGFR2) IIIb. FGFR2IIIb-SRE-HEK293 monoclone were grown in DMEM medium with 10% FBS and then seed in 40,000/well and starve in DMEM+0.5% overnight. Then added various concentration mAb1 for 30 min, followed by incubation with FGF7 protein for 4 h. The luciferase signal was detected by TECAN. This inhibition data of mAb1 was processed using Graphpad Prism and the data was shown in FIG. 7. FGFR2 signaling pathway was inhibited by mAb1 in a concentration-dependent manner in FGFR2b overexpression cells.

### Example 5. In Vivo Antitumor Activity of Antibody in Tumor Mice Models

Cell Line-Derived Xenograft (CDX) mice models were established by first culturing the SNU16 cells in vitro, and then inoculating the SNU16 cells subcutaneously into the dorsal flanks of mice at 3×10⁶ cells/100 μL mixed with 50% Matrigel/mouse.

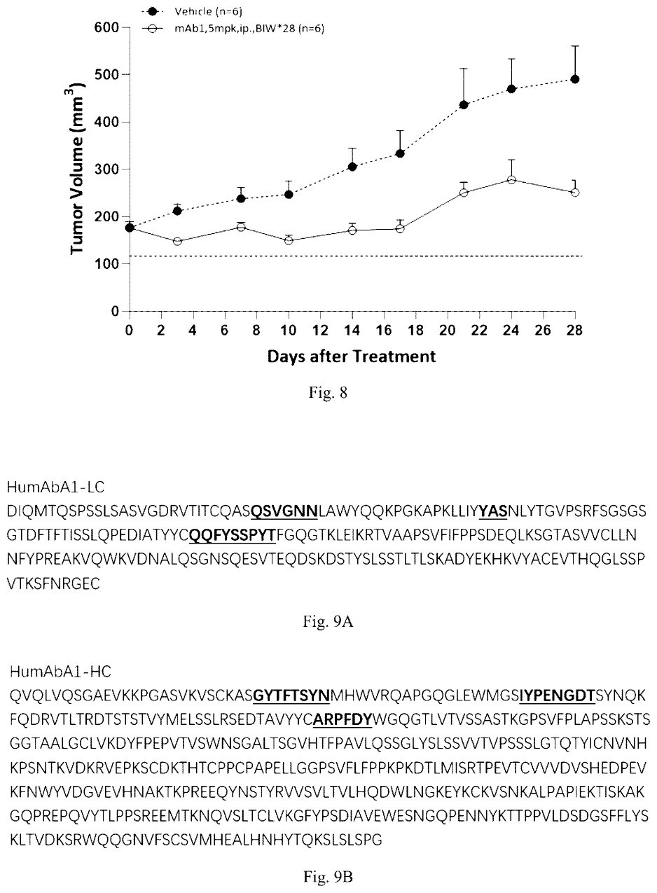

Tumor nodules were measured in two dimensions with callipers and the tumor volume was calculated using the following formula: tumor volume=(length×width2)×0.52. When the tumor volume reached 150-200 mm³, tumor-bearing mice were randomized into treatment groups. Mice were then treated with either isotype control (e.g., IgG1) or tested antibodies (e.g., FPA144, mAb1) twice a week from the day after randomisation. The tumor volume and body weight of the mice were measured twice weekly and the raw data were recorded. Tumor growth inhibition from start of treatment was assessed by comparing the mean change in tumor volume between the control and treated groups. The calculation was based on the geometric or arithmetic mean of relative tumor volume (RTV) in each group. RTV was calculated by dividing the tumor volume on the treatment day with the initial tumor volume. The in vivo tumor growth curve of SNU16 cells with mAb1 treatment were shown in FIG. 8, mAb1 shows better anti-tumor activity.

### Example 6. Humanization of mAb 1

Humanization of mAb 1 was designed, constructed and expressed using standard methods of molecular biology. In brief, the CDRs of mAb 1 was grafted into a human acceptor framework. Then at framework position where the computer model suggests significant contact with CDRs, the amino acid residues from mouse antibody were substituted for human framework amino acid residues, including M481 and V68A of the heavy chain and 49F of the light chain, using Kabat numbering. This provided for the humanized antibody of mAb 1, designated as humAb 1. The amino acids NG in the CDR2 of the heavy chain of humAb 1 was further substituted.

### Example 7. ADCC Enhancement of the Antibodies

Antibody dependent cellular cytotoxicity (ADCC), also known as antibody-dependent cell mediated cytotoxicity, is an immune mechanism by which the effector cells expressing Fc receptors recognize and kill target cells expressing tumor-derived or pathogen-derived antigens on their surface. Given that increased IgG binding affinity due to CD16A polymorphisms can enhance ADCC and improve clinical outcomes, a strategy has emerged to improve therapeutic mAb function by using the 1,6-fucosyltransferase knockout (FUT8−/−) CHOK1 cell (Wuxi Biologies, China, Shanghai) as the host cell line to produce fucose-free antibodies (afucosylated antibodies). This provided for the ADCC enhancement of humAb 1, designated as humAbA1. Another strategy has emerged to improve therapeutic mAb function by modifying the Fc portion of tumor-targeting antibodies. This has been achieved through amino acid substitutions. This provided for the ADCC enhancement of humAb 1, designated as humAbA2.

The afucosylated antibodies are purified by Protein A and SEC-HPLC and dialysis to exchange into formulation buffer and store at −80° C. The glycan of the purified afucosylated antibodies were analyzed using LC-MS. The mass of each peak is determined and used to identify each glycan, and the result demonstrates that each of the afucosylated antibodies is almost 100% afucosylated. It is expected that the afucosylated antibody would provide at least comparable in vitro or in vivo activities as compared to their fucosylated counterparts.

Amino acid sequences of the entire mature humAbA1 light chain and heavy chain with human IgG1 are shown in FIG. 9.

### Example 8. Binding Characteristics of the Antibodies

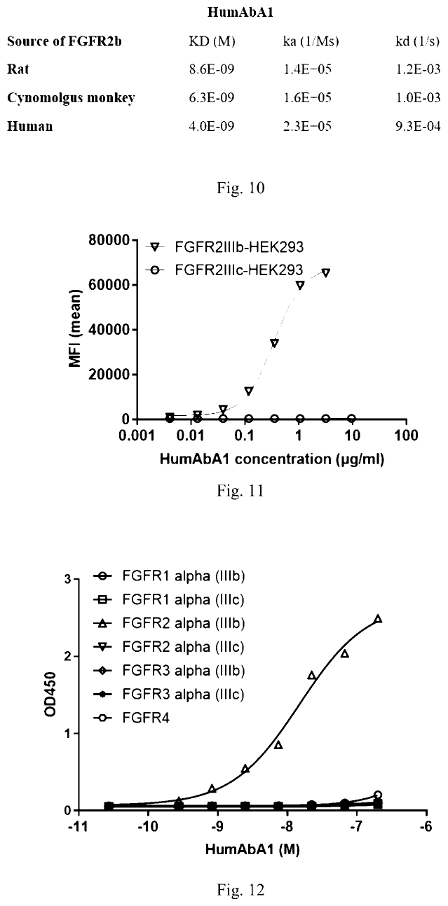

The binding of antibodies to human FGFR2b antigen was determined by Biolayer Interferometry (BLI) (OCTET). Briefly, Protein A Biosensor was firstly prewet by a 10 min injection of PBST (0.05% Tween 20). Antibodies were diluted in PBST-BSA (0.05% Tween, 0.1% BSA) and injected at serial concentration (0, 1.56, 3.12, 6.25, 12.5, 25, 50 nM) and surface regeneration of the Protein A Biosensor were included in each running cycle. The association constant, dissociation constant was calculated with OCTET RED384 evaluation software (version 1.0). As shown in FIG. 10, humAbA1 demonstrated sub-nanomolar equilibrium dissociation constants (K_{D}) for rat, cynomolgus monkey, and human FGFR2b ECD with comparable associate and dissociate constants (Kon and K_{off}). Based on these data, rat and cynomolgus monkey were deemed appropriate species in which to perform toxicology studies with humAbA1.

To confirm that the selected antibody can bind to the endogenous forms of FGFR2b on the cell membrane, flow cytometry was performed using human HEK293 cells which transfected with genes for FGFR2IIIc and FGFR2IIIb. FGFR2IIIb-HEK293 or FGFR2IIIc-HEK293 monoclone were grown in DMEM medium with 10% FBS and then seed in 100,000/well and wash with PBS+0.5% FBS. Then added various concentration mAbs for 30 min, followed by incubation with fluorescent coupled anti human IgG Fc secondary antibody for 30 min. The fluorescence signal was detected by Cytoflex. This binding activity data of mAb1 was processed using Graphpad Prism and the data was shown in FIG. 11. As seen in Figure. 11, humAbA1 bind to the FGFR2IIIb-transfected cells, as expected from their epitopes, and cannot bind to FGFR2IIIc transfected cells.

The binding specificity of humAbA1 with various FGFR family member, FGFR1b, FGFR1c, FGFR2b, FGFR2c, FGFR3b, FGFR3c and FGFR4 was characterized with ELISA assay. In brief, 96-well ELISA plate were coated with about 50 ng/well 0.5 mg/ml human FGFR1 alpha (IIIb)-His, human FGFR1/CD331 protein-His, human FGFR2 alpha (IIIb) Protein, human FGFR2 alpha (IIIc) Protein, human FGFR3 alpha (IIIb)-His, human FGFR3 alpha (IIIc)-His, and human FGFR4/CD334-His in PH 9.6 carbonate buffer solution overnight. Then the plate was blocked with 2% BSA in PBST (PBS with 0.05% Tween20) and incubation with antibody samples for 60 min at room temperature, and then washed once in PBST and followed by incubation with mAb1 (0.2 μm first hole, 3 times down 7 steps, the last step down 10 times) at room temperature. Then washed once in PBST and followed by incubation with Peroxidase-conjugated AffiniPure F (AB′) 2 Fragment Goat Anti-Human IgG (Jackson Immuno Research, #109-036-098) conjugate for 30 min HRP activity was detected with TMB Substrate (Solarbio, #PR1200) and the reaction was stopped with stop solution (Solarbio, #C1058). The plate was read at 450 nm. The data is shown in FIG. 12. According to the result of ELISA analysis, humAbA1 specifically binds to FGFR2b, and it does not bind to any other FGFR family members.

### Example 9. In Vitro Activity

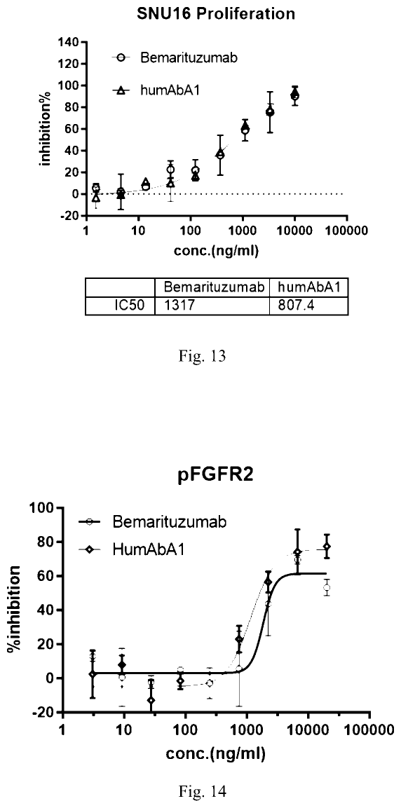

The inhibition activity of antibody on ligand-induced cell proliferation was done in SNU 16 cell clones. Cells were seeded in 96-well plates at 5,000 cell/well in RPMI1640 medium containing 0.5% fetal bovine serum. Starve the cell for 16 hours/overnight. SNU-16 cells were treated with varying concentrations of antibodies diluted in RPMI media with 0.5% FBS for 30 minutes. SNU-16 cells were then treated with 100 ng/ml FGF7 with 1 μg/mL heparin (final concentrations) diluted in RPMI media with 0% FBS, and incubated at 37° C. with 5% CO₂ for 4 days. Add 50 μL CTG, read luminescence as T144H. This inhibition activity data of humAbA1 was processed using Graphpad Prism and the data was shown in FIG. 13. In the presence of increasing concentrations of humAbA1, FGF7-induced (FIG. 5) cell proliferation was inhibited in a concentration-dependent manner in SNU-16 cells, the potency of humAbA1 was better than Bemarituzumab.

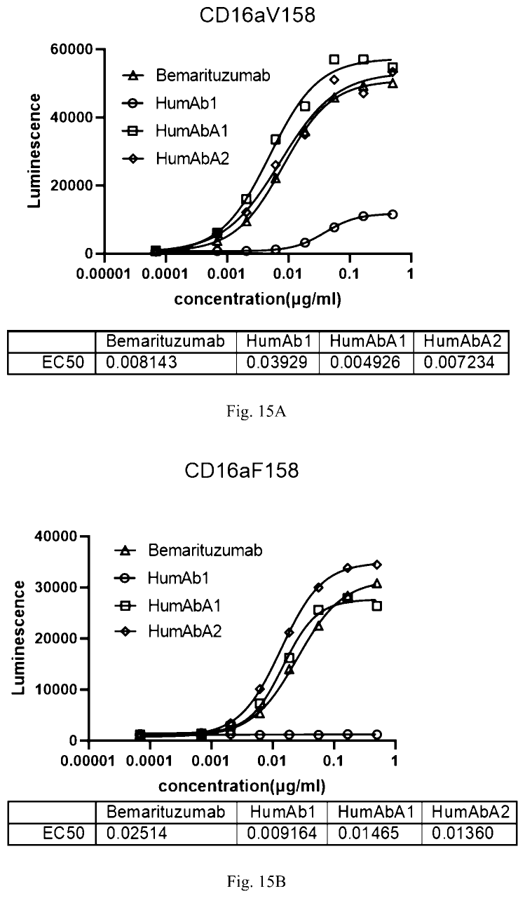

The inhibition of antibody on FGFR2 phosphorylation was assessed in the SNU-16 human gastric cancer cell line. In the presence of increasing concentrations of mAbs, FGF7-induced FGFR2 phosphorylation was inhibited in a concentration-dependent manner in SNU-16 cells (FIG. 14).

In vitro assays to determine the ADCC activity of antibody was performed. ADCC reporter cell lines Jurkat-Luc NFAT-CD16a-V158 and Jurkat-Luc NFAT-CD16a-F158 were developed based on Jurkat cells which stably integrated high-affinity CD16a (V158 allotype) or low-affinity CD16 (F158), respectively, and luciferase gene under the control of the NFAT (Nuclear factor of activated T-cells) response element. Reporter cells were plated in 96 well plate with 1E5 cells per well, followed by adding serially diluted mAbs for 30 min at 37° C. FGFR2b-expressing cell line KATOIII as target cells were co-incubated with effector cells Jurkat-Luc NFAT-CD16a-V158 or Jurkat-Luc NFAT-CD16a-F158 with E: T=5:1 for 6h. Luminescence was measured after 5-30 min incubation with substrate Nano-glo by microplate reader. Fitting curves were graphed as RLU versus Log10 [antibody] using GraphPad Prism® software. ADCC activity was determined by calculating EC50 of antibody response and maximum response efficacy as shown in FIG. 15. humAbA1 exhibits superior ADCC activity compared to Bemarituzumab.

### Example 10. In Vivo Antitumor Activity of Antibody in Tumor Mice Models

Cell Line-Derived Xenograft (CDX) mice models were established by first culturing the SNU-16 or OCUM-2M cells in vitro, and then inoculating the SNU-16/OCUM-2M cells subcutaneously into the dorsal flanks of mice at 3×10^{6~5}×10⁶ cells/100 μL mixed with 50% Matrigel/mouse.

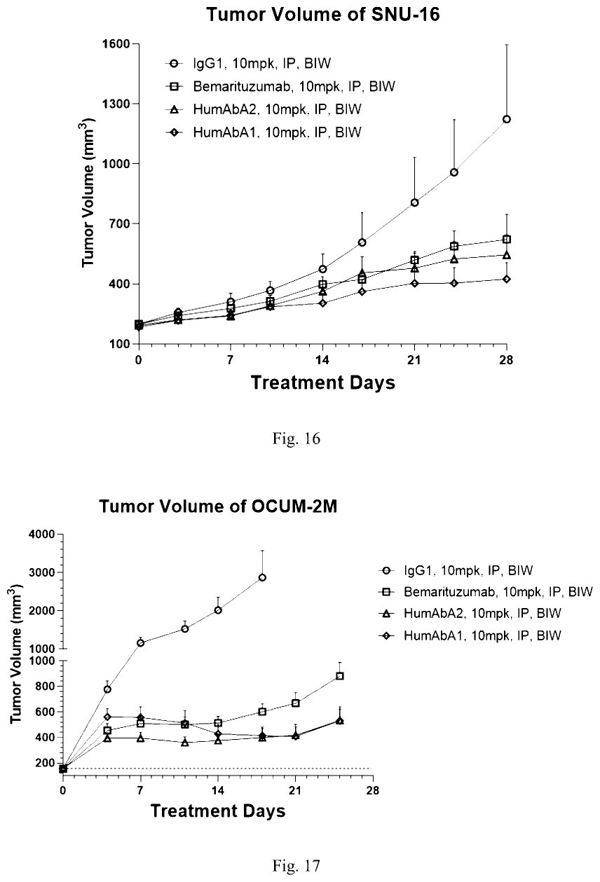

Tumor nodules were measured in two dimensions with callipers and the tumor volume was calculated using the following formula: tumor volume=(length×width2)×0.52. When the tumor volume reached 150-200 mm³, tumor-bearing mice were randomized into treatment groups. Mice were then treated with either isotype control (e.g., IgG1) or tested antibodies (e.g., Bemarituzumab, humAbA1, humAbA2) twice a week from the day after randomisation. The tumor volume and body weight of the mice were measured twice weekly and the raw data were recorded. Tumor growth inhibition from start of treatment was assessed by comparing the mean change in tumor volume between the control and treated groups. The calculation was based on the geometric or arithmetic mean of relative tumor volume (RTV) in each group. RTV was calculated by dividing the tumor volume on the treatment day with the initial tumor volume. The in vivo tumor growth curve of SNU-16 cells with antibodies treatment were shown in FIG. 16, humAbA1 and humAbA2 shows better anti-tumor activity than Bemarituzumab. The in vivo tumor growth curve of OCUM-2M cells with antibodies treatment were shown in FIG. 17, humAbA1 and humAbA2 shows better anti-tumor activity than Bemarituzumab.

### REFERENCE LIST

1 Katoh M and Nakagama H. FGF receptors: cancer biology and therapeutics. Med Res Rev 2014; 34:280-300.
2 Eswarakumar V P, Lax I, Schlessinger J. Cellular signaling by fibroblast growth factor receptors. Cytokine Growth Factor Rev 2005; 16:139-49.
3 Turner N, Grose R. Fibroblast growth factor signalling: from development to cancer. Nat Rev Cancer 2010; 10:116-29.
4 Beenken A, Mohammadi M. The FGF family: biology, pathophysiology and therapy. Nat Rev Drug Discov 2009; 8:235-53.
Ornitz D M, Xu J, Colvin J S, et al. Receptor specificity of the fibroblast growth factor family. J Biol Chem 1996; 271:15292-7.
6 Zhang X, IbrahimiOA,OlsenSK, Umemori H,Mohammadi M, OrnitzDM. Receptor specificity of the fibroblast growth factor family. The complete mammalian FGF family. J Biol Chem 2006; 281:15694-700.
7 Bai, Ailin, et al. “GP369, an FGFR2-IIIb-Specific Antibody, Exhibits Potent Antitumor Activity against Human Cancers Driven by Activated FGFR2 SignalingIn vivo Efficacy of GP369 in FGFR2-Amplified Tumors.” Cancer research 70.19 (2010): 7630-7639. Easton D F, Pooley K A, Dunning A M, et al. Genome-wide association study identifies novel breast cancer susceptibility loci. Nature 2007; 447:1087-93.
8 Hunter D J, Kraft P, Jacobs K B, et al. A genome-wide association study identifies alleles in FGFR2 associated with risk of sporadic postmenopausal breast cancer. Nat Genet 2007; 39:870-4.
9 Meyer K B, Maia A T, O'Reilly M, et al. Allele-specific up-regulation of FGFR2 increases susceptibility to breast cancer. PLOS Biol 2008; 6: e108.
10 Davies H, Hunter C, Smith R, et al. Somatic mutations of the protein kinase gene family in human lung cancer. Cancer Res 2005; 65:7591-5.
11 Ding L, Getz G, Wheeler D A, et al. Somatic mutations affect key pathways in lung adenocarcinoma. Nature 2008; 455:1069-75.
12 Dutt A, Salvesen H B, Chen T H, et al. Drug-sensitive FGFR2 mutations in endometrial carcinoma. Proc Natl Acad Sci USA 2008; 105:8713-7.
13 Greenman C, Stephens P, Smith R, et al. Patterns of somatic mutation in human cancer genomes. Nature 2007; 446:153-8.
14 Pollock P M, Gartside M G, Dejeza L C, et al. Frequent activating FGFR2 mutations in endometrial carcinomas parallel germline mutations associated with craniosynostosis and skeletal dysplasia syndromes. Oncogene 2007; 26:7158-62.
15 Jang J H, Shin K H, Park J G. Mutations in fibroblast growth factor receptor 2 and fibroblast growth factor receptor 3 genes associated with human gastric and colorectal cancers. Cancer Res 2001; 61:3541-3.
16 Heiskanen M, Kononen J, Barlund M, et al. CGH, cDNA and tissue microarray analyses implicate FGFR2 amplification in a small subset of breast tumors. Anal Cell Pathol 2001; 22:229-34.
17 Adnane J, Gaudray P, Dionne C A, et al. BEK and FLG, two receptors to members of the FGF family, are amplified in subsets of human breast cancers. Oncogene 1991; 6:659-63.
18 Turner N, Lambros M B, Horlings H M, et al. Integrative molecular profiling of triple negative breast cancers identifies amplicon drivers and potential therapeutic targets. Oncogene 2010; 29:2013-23.
19 Hara T, Ooi A, Kobayashi M, Mai M, Yanagihara K, Nakanishi I. Amplification of c-myc, K-sam, and c-met in gastric cancers: detection by fluorescence in situ hybridization. Lab Invest 1998; 78:1143-53.
20 Mor O, Ranzani G N, Ravia Y, et al. DNA amplification in human gastric carcinomas. Cancer Genet Cytogenet 1993; 65:111-4.
21 Tsujimoto H, Sugihara H, Hagiwara A, Hattori T. Amplification of growth factor receptor genes and DNA ploidy pattern in the progression of gastric cancer. Virchows Arch 1997; 431:383-9.
22 Yoshida T, Sakamoto H, Terada M. Amplified genes in cancer in upper digestive tract. Semin Cancer Biol 1993; 4:33-40.
23 Yamayoshi T, Nagayasu T, Matsumoto K, Abo T, Hishikawa Y, Koji T. Expression of keratinocyte growth factor/fibroblast growth factor-7 and its receptor in human lung cancer: correlation with tumour proliferative activity and patient prognosis. J Pathol 2004; 204:110-8.
24 Cho K, Ishiwata T, Uchida E, et al. Enhanced expression of keratinocyte growth factor and its receptor correlates with venous invasion in pancreatic cancer. Am J Pathol 2007; 170:1964-74.
25 Toyokawa T, Yashiro M, Hirakawa K. Co-expression of keratinocyte growth factor and K-sam is an independent prognostic factor in gastric carcinoma. Oncol Rep 2009; 21:875-80.
26 Gartside M G, Chen H, Ibrahimi O A, et al. Loss-of-function fibroblast growth factor receptor-2 mutations in melanoma. Mol Cancer Res 2009; 7:41-54.
27 Diez de Medina S G, Chopin D, El Marjou A, et al. Decreased expression of keratinocyte growth factor receptor in a subset of human transitional cell bladder carcinomas. Oncogene 1997; 14:323-30.
28 Giri D, Ropiquet F, Ittmann M. Alterations in expression of basic fibroblast growth factor (FGF) 2 and its receptor FGFR-1 in human prostate cancer. Clin Cancer Res 1999; 5:1063-71.
29 Ricol D, Cappellen D, El Marjou A, et al. Tumour suppressive properties of fibroblast growth factor receptor 2-IIIb in human bladder cancer. Oncogene 1999; 18:7234-43.
30 Zhang Y, Wang H, Toratani S, et al. Growth inhibition by keratinocyte growth factor receptor of human salivary adenocarcinoma cells through induction of differentiation and apoptosis. Proc Natl Acad Sci USA 2001; 98:11336-40.
31 Grose R, Fantl V, Werner S, et al. The role of fibroblast growth factor receptor 2b in skin homeostasis and cancer development. EMBO J 2007; 26:1268-78.
32 Lin Y, Liu G, Zhang Y, et al. Fibroblast growth factor receptor 2 tyrosine kinase is required for prostatic morphogenesis and the acquisition of strict androgen dependency for adult tissue homeostasis. Development 2007; 134:723-34.
33 Grilo A. L., Mantalaris A. (2019) The increasingly human and profitable monoclonal antibody market. Trends Biotechnol., 37, 9-16.
34 Liu J. K. H. (2014) The history of monoclonal antibody development-progress, remaining challenges and future innovations. Ann. Med. Surg., 3, 113-116.
35 Chothia, C., & Lesk, A. M. (1987). Canonical structures for the hypervariable regions of T cell αβ receptors. J Mol Biol, 196, 901-17.
36 Chothia, C., Lesk, A. M., Tramontano, A., Levitt, M., Smith-Gill, S. J., Air, G., . . . & Poljak, R. J. (1989). Conformations of immunoglobulin hypervariable regions. Nature, 342 (6252), 877-883.
37 Kabat, E. A., Wu, T. T., Perry, H. M., Gottesmann, K. S., & Foeller, C. (1991). Sequences of Proteins of Immunological Interest. 5th edn. National Institutes of Health Publication No. 91-3242. National Institutes of Health, Bethesda, M D.
38 Al-Lazikani, B., Lesk, A. M., & Chothia, C. (1997). Standard conformations for the canonical structures of immunoglobulins. Journal of molecular biology, 273 (4), 927-948.
39 Lefranc, M. P., Pommié, C., Ruiz, M., Giudicelli, V., Foulquier, E., Truong, L., . . . & Lefranc, G. (2003). IMGT unique numbering for immunoglobulin and T cell receptor variable domains and Ig superfamily V-like domains. Developmental & Comparative Immunology, 27 (1), 55-77.
40 Tonegawa, S. (1983). Somatic generation of antibody diversity. Nature, 302 (5909), 575-581.
41 Xu, J. L., & Davis, M. M. (2000). Diversity in the CDR3 region of V H is sufficient for most antibody specificities. Immunity, 13 (1), 37-45.
42 Schier, R., McCall, A., Adams, G. P., Marshall, K. W., Merritt, H., Yim, M., . . . & Marks, J. D. (1996). Isolation of picomolar affinity anti-c-erbB-2 single-chain Fv by molecular evolution of the complementarity determining regions in the center of the antibody binding site. Journal of molecular biology, 263 (4), 551-567.
43 Köhler, G., & Milstein, C. (1975). Continuous cultures of fused cells secreting antibody of predefined specificity. nature, 256 (5517), 495-497.
44 Kennet, R. H., McKeam, T. J., & Bechtol, K. B. (1980). Methods for production and characterization of monoclonal antibodies. Monoclonal Antibodies (Appendix), R H Kennet, T J Mckeam, and K B Bechtol, eds., Plenum, New York.
45 Batzer, M. A., Arcot, S. S., Phinney, J. W., Alegria-Hartman, M., Kass, D. H., & Milligan, S. M., et al. (1996). Genetic variation of recent alu insertions in human populations. Journal of Molecular Evolution, 42 (1), 22-29.
46 Ohtsuka, E., Matsuki, S., Ikehara, M., Takahashi, Y., & Matsubara, K. (1985). An alternative approach to deoxyoligonucleotides as hybridation probes by insertion of deoxyinosine at ambiguous codon positions. Journal of Biological Chemistry, 260 (5), 2605-2608.
47 Cassol, S. A., Lapointe, N., Salas, T., Hankins, C., & Charest, J. (1992). Diagnosis of vertical hiv-1 transmission using the polymerase chain reaction and dried blood spot specimens. J Acquir Immune Defic Syndr, 5 (2), 113-119.
48 Rossolini, G. M., Cresti, S., Ingianni, A., Cattani, P., & Satta, G. (1994). Use of deoxyinosine-containing primers vs degenerate primers for polymerase chain reaction based on ambiguous sequence information. Molecular & Cellular Probes, 8 (2), 91.
49 “Sequences of Proteins of Immunological Interest,” 5th Ed. Public Health Service, National Institutes of Health, Bethesda, M D (“Kabat” numbering scheme),
50 Al-Lazikani et al., (1997) J M B 273,927-948 (“Chothia” numbering scheme);
51 MacCallum et al., J. Mol. Biol. 262:732-745 (1996), “Antibody-antigen interactions: Contact analysis and binding site topography,” J. Mol. Biol. 262, 732-745.” (“Contact” numbering scheme);
52 Lefranc M P et al., “IMGT unique numbering for immunoglobulin and T cell receptor variable domains and Ig superfamily V-like domains,” Dev Comp Immunol, 2003 January;27 (1): 55-77 (“IMGT” numbering scheme);
53 Honegger A and Plückthun A, “Yet another numbering scheme for immunoglobulin variable domains: an automatic modeling and analysis tool,” J Mol Biol, 2001 Jun. 8;309 (3): 657-70, (“Aho” numbering scheme);
54 Martin et al., “Modeling antibody hypervariable loops: a combined algorithm,” PNAS, 1989, 86 (23): 9268-9272, (“AbM” numbering scheme).
55 Ward, E. Sally, et al. “Binding activities of a repertoire of single immunoglobulin variable domains secreted from Escherichia coli.” Nature 341.6242 (1989): 544-546.
56 Bird, Robert E., et al. “Single-chain antigen-binding proteins.” Science 242.4877 (1988): 423-426.
57 Pope, Matthew E., et al. “Anti-peptide antibody screening: selection of high affinity monoclonal reagents by a refined surface plasmon resonance technique.” Journal of immunological methods 341.1-2 (2009): 86-96.

What is claimed is:

1. An antibody, comprising a heavy chain variable region, wherein the heavy chain variable region comprises at least one complementarity determining region (CDR), wherein the CDR that is farthest from the N terminus is selected from SEQ ID NOs: 61, 67, 73, 79, 85, 89, 95, 101, 104, 112, 116, 121, 125, 128, 131, 136, 138, 142, 144, 147, 151, 154, and 158.

2. The antibody of claim 1, wherein the heavy chain variable region comprises three CDRs, which are hCDR1, hCDR2, and hCDR3 from N terminus to C terminus, wherein the hCDR1 is selected from SEQ ID NOs: 59, 65, 71, 77, 83, 88, 93, 103, 110, 119, 135, 143, 146, 150, and 152; the hCDR2 is selected from SEQ ID NOs: 60, 66, 72, 78, 84, 94, 111, 118, 120, 124, 141, and 153; and the hCDR3 is selected from SEQ ID NOs: 61, 67, 73, 79, 85, 89, 95, 101, 104, 112, 116, 121, 125, 128, 131, 136, 138, 142, 144, 147, 151, 154 and 158.

3. The antibody of claim 2, wherein the hCDR1, hCDR2, and hCDR3 comprise:
a. SEQ ID NO: 59, SEQ ID NO: 60, SEQ ID NO: 61, respectively;
b. SEQ ID NO: 65, SEQ ID NO: 66, SEQ ID NO: 67, respectively;
c. SEQ ID NO: 71, SEQ ID NO: 72, SEQ ID NO: 73, respectively;
d. SEQ ID NO: 77, SEQ ID NO: 78, SEQ ID NO: 79, respectively;
e. SEQ ID NO: 83, SEQ ID NO: 84, SEQ ID NO: 85, respectively;
f. SEQ ID NO: 88, SEQ ID NO: 84, SEQ ID NO: 89, respectively;
g. SEQ ID NO: 93, SEQ ID NO: 94, SEQ ID NO: 95, respectively;
h. SEQ ID NO: 59, SEQ ID NO: 78, SEQ ID NO: 79, respectively;
i. SEQ ID NO: 93, SEQ ID NO: 94, SEQ ID NO: 101, respectively;
j. SEQ ID NO: 103, SEQ ID NO: 94, SEQ ID NO: 104, respectively;
k. SEQ ID NO: 59, SEQ ID NO: 78, SEQ ID NO: 79, respectively;
l. SEQ ID NO: 110, SEQ ID NO: 111, SEQ ID NO: 112, respectively;
m. SEQ ID NO: 88, SEQ ID NO: 84, SEQ ID NO: 116, respectively;
n. SEQ ID NO: 93, SEQ ID NO: 94, SEQ ID NO: 101, respectively;
o. SEQ ID NO: 93, SEQ ID NO: 118, SEQ ID NO: 101, respectively;
p. SEQ ID NO: 119, SEQ ID NO: 120, SEQ ID NO: 121, respectively;
q. SEQ ID NO: 119, SEQ ID NO: 124, SEQ ID NO: 125, respectively;
r. SEQ ID NO: 77, SEQ ID NO: 78, SEQ ID NO: 128, respectively;
s. SEQ ID NO: 83, SEQ ID NO: 94, SEQ ID NO: 131, respectively;
t. SEQ ID NO: 135, SEQ ID NO: 84, SEQ ID NO: 136, respectively;
u. SEQ ID NO: 88, SEQ ID NO: 84, SEQ ID NO: 138, respectively;
v. SEQ ID NO: 83, SEQ ID NO: 141, SEQ ID NO: 142, respectively;
w. SEQ ID NO: 143, SEQ ID NO: 84, SEQ ID NO: 144, respectively;
x. SEQ ID NO: 146, SEQ ID NO: 94, SEQ ID NO: 147, respectively;
y. SEQ ID NO: 119, SEQ ID NO: 120, SEQ ID NO: 121, respectively;
z. SEQ ID NO: 119, SEQ ID NO: 124, SEQ ID NO: 125, respectively;
aa. SEQ ID NO: 150, SEQ ID NO: 84, SEQ ID NO: 151, respectively;
bb. SEQ ID NO: 152, SEQ ID NO: 153, SEQ ID NO: 154, respectively; or
cc. SEQ ID NO: 88, SEQ ID NO: 94, SEQ ID NO: 158, respectively.

4. The antibody of any one of claims 1-3, wherein the heavy chain variable region comprises four framework regions (FRs), which are hFR1, hFR2, hFR3, and hFR4 from N terminus to C terminus, wherein the hFR1 is selected from SEQ ID NOs: 160, 168, 175, 188, 194, 204, 210, 224, 233, 284, 259, 265, 268, 271, 274, 281, 295, and 305; the hFR2 is selected from SEQ ID NOs: 161, 169, 176, 183, 205, 221, 225, 237, 245, 251, 261, 266, 275, 282, and 296; the hFR3 is selected from SEQ ID NOs: 162, 170, 177, 184, 189, 195, 200, 206, 211, 216, 226, 231, 234, 239, 242, 246, 252, 254, 260, 262, 267, 272, 276, 297, and 306; and the hFR4 is selected from SEQ ID NOs: 163, 178, 190, 196, 212, 217, 227, 255, 269, and 298.

5. The antibody of claim 4, wherein the hFR1, hFR2, hFR3, and hFR4 comprise:
a. SEQ ID NO: 160, SEQ ID NO: 161, SEQ ID NO: 162, SEQ ID NO: 163, respectively;
b. SEQ ID NO: 168, SEQ ID NO: 169, SEQ ID NO: 170, SEQ ID NO: 163, respectively;
c. SEQ ID NO: 175, SEQ ID NO: 176, SEQ ID NO: 177, SEQ ID NO: 178, respectively;
d. SEQ ID NO: 160, SEQ ID NO: 183, SEQ ID NO: 184, SEQ ID NO: 178, respectively;
e. SEQ ID NO: 188, SEQ ID NO: 169, SEQ ID NO: 189, SEQ ID NO: 190, respectively;
f. SEQ ID NO: 194, SEQ ID NO: 169, SEQ ID NO: 195, SEQ ID NO: 196, respectively;
g. SEQ ID NO: 194, SEQ ID NO: 169, SEQ ID NO: 200, SEQ ID NO: 163, respectively;
h. SEQ ID NO: 204, SEQ ID NO: 205, SEQ ID NO: 206, SEQ ID NO: 178, respectively;
i. SEQ ID NO: 210, SEQ ID NO: 169, SEQ ID NO: 211, SEQ ID NO: 212, respectively;
j. SEQ ID NO: 194, SEQ ID NO: 169, SEQ ID NO: 216, SEQ ID NO: 217, respectively;
k. SEQ ID NO: 204, SEQ ID NO: 221, SEQ ID NO: 206, SEQ ID NO: 178, respectively;
l. SEQ ID NO: 224, SEQ ID NO: 225, SEQ ID NO: 226, SEQ ID NO: 227, respectively;
m. SEQ ID NO: 194, SEQ ID NO: 169, SEQ ID NO: 231, SEQ ID NO: 163, respectively;
n. SEQ ID NO: 233, SEQ ID NO: 169, SEQ ID NO: 234, SEQ ID NO: 178, respectively;
o. SEQ ID NO: 210, SEQ ID NO: 237, SEQ ID NO: 211, SEQ ID NO: 212, respectively;
p. SEQ ID NO: 194, SEQ ID NO: 169, SEQ ID NO: 239, SEQ ID NO: 190, respectively;
q. SEQ ID NO: 194, SEQ ID NO: 169, SEQ ID NO: 242, SEQ ID NO: 178, respectively;
r. SEQ ID NO: 284, SEQ ID NO: 183, SEQ ID NO: 184, SEQ ID NO: 163, respectively;
s. SEQ ID NO: 194, SEQ ID NO: 245, SEQ ID NO: 246, SEQ ID NO: 163, respectively;
t. SEQ ID NO: 188, SEQ ID NO: 251, SEQ ID NO: 252, SEQ ID NO: 163, respectively;
u. SEQ ID NO: 188, SEQ ID NO: 169, SEQ ID NO: 254, SEQ ID NO: 255, respectively;
v. SEQ ID NO: 259, SEQ ID NO: 169, SEQ ID NO: 260, SEQ ID NO: 190, respectively;
w. SEQ ID NO: 194, SEQ ID NO: 261, SEQ ID NO: 262, SEQ ID NO: 163, respectively;
x. SEQ ID NO: 265, SEQ ID NO: 266, SEQ ID NO: 267, SEQ ID NO: 163, respectively;
y. SEQ ID NO: 268, SEQ ID NO: 169, SEQ ID NO: 239, SEQ ID NO: 269, respectively;
z. SEQ ID NO: 194, SEQ ID NO: 169, SEQ ID NO: 200, SEQ ID NO: 190, respectively;
aa. SEQ ID NO: 271, SEQ ID NO: 169, SEQ ID NO: 272, SEQ ID NO: 190, respectively;
bb. SEQ ID NO: 274, SEQ ID NO: 275, SEQ ID NO: 276, SEQ ID NO: 178, respectively; or
cc. SEQ ID NO: 281, SEQ ID NO: 282, SEQ ID NO: 254, SEQ ID NO: 163, respectively,
dd. SEQ ID NO: 295, SEQ ID NO: 296, SEQ ID NO: 297, SEQ ID NO: 298, respectively;
ee. SEQ ID NO: 305, SEQ ID NO: 296, SEQ ID NO: 306, SEQ ID NO: 298, respectively;
ff. SEQ ID NO: 295, SEQ ID NO: 296, SEQ ID NO: 306, SEQ ID NO: 298, respectively;
gg. SEQ ID NO: 305, SEQ ID NO: 296, SEQ ID NO: 297, SEQ ID NO: 298, respectively;

6. The antibody of any one of claims 4 and 5, wherein the CDRs and FRs in the heavy chain variable region are operably connected in the order of hFR1-hCDR1-hFR2-hCDR2-hFR3-hCDR3-hFR4 from N terminus to C terminus.

7. The antibody of any one of claims 1-6, further comprising a heavy chain constant region, wherein the heavy chain constant region comprises three domains, CH1, CH2, and CH3, from N-terminus to C terminus, and wherein the CH1, CH2, and CH3 are
a. SEQ ID NO. 307, SEQ ID NO. 309, and SEQ ID NO. 310, respectively; or
b. SEQ ID NO: 307, SEQ ID NO. 312, and SEQ ID NO. 313, respectively.

8. The antibody of any one of claims 1-7, wherein the heavy chain variable region comprises an amino acid sequence with at least 80%, 85%, 90%, 95%, 96%, 97%, 98%, 99%, or 100% identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 285, 287, 290, and 291.

9. The antibody of any one of claims 1-8, wherein the heavy chain variable region comprises an amino acid sequence of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 285, 287, 290, and 291.

10. An antibody, comprising a light chain variable region, wherein the light chain variable region comprises at least one CDR, wherein the CDR that is farthest from the N terminus is selected from SEQ ID NOs: 64, 70, 76, 82, 87, 92, 98, 100, 102, 107, 109, 115, 117, 123, 127, 130, 134, 137, 140, 145, 157, and 159.

11. The antibody of claim 10, wherein the light chain variable region comprises three CDRs, which are ICDR1, 1CDR2, and ICDR3 from N terminus to C terminus, wherein the 1CDR1 is selected from SEQ ID NOs: 62, 68, 74, 80, 86, 90, 96, 99, 105, 108, 113, 122, 126, 129, 132, 148, 149, and 155; the ICDR2 is selected from SEQ ID NOs: 63, 69, 75, 81, 91, 97, 106, 114, 133, 139, and 156; and the 1CDR3 is selected from SEQ ID NOs: 64, 70, 76, 82, 87, 92, 98, 100, 102, 107, 109, 115, 117, 123, 127, 130, 134, 137, 140, 145, 157, and 159.

12. The antibody of claim 11, wherein the ICDR1, ICDR2, and ICDR3 comprise:
a. SEQ ID NO: 62, SEQ ID NO: 63, SEQ ID NO: 64, respectively;
b. SEQ ID NO: 68, SEQ ID NO: 69, SEQ ID NO: 70, respectively;
c. SEQ ID NO: 74, SEQ ID NO: 75, SEQ ID NO: 76, respectively;
d. SEQ ID NO: 80, SEQ ID NO: 81, SEQ ID NO: 82, respectively;
e. SEQ ID NO: 86, SEQ ID NO: 69, SEQ ID NO: 87, respectively;
f. SEQ ID NO: 90, SEQ ID NO: 91, SEQ ID NO: 92, respectively;
g. SEQ ID NO: 96, SEQ ID NO: 97, SEQ ID NO: 98, respectively;
h. SEQ ID NO: 99, SEQ ID NO: 69, SEQ ID NO: 100, respectively;
i. SEQ ID NO: 96, SEQ ID NO: 97, SEQ ID NO: 102, respectively;
j. SEQ ID NO: 105, SEQ ID NO: 106, SEQ ID NO: 107, respectively;
k. SEQ ID NO: 108, SEQ ID NO: 69, SEQ ID NO: 109, respectively;
l. SEQ ID NO: 113, SEQ ID NO: 114, SEQ ID NO: 115, respectively;
m. SEQ ID NO: 105, SEQ ID NO: 106, SEQ ID NO: 117, respectively;
n. SEQ ID NO: 96, SEQ ID NO: 97, SEQ ID NO: 102, respectively;
o. SEQ ID NO: 96, SEQ ID NO: 97, SEQ ID NO: 102, respectively;
p. SEQ ID NO: 122, SEQ ID NO: 69, SEQ ID NO: 123, respectively;
q. SEQ ID NO: 126, SEQ ID NO: 69, SEQ ID NO: 127, respectively;
r. SEQ ID NO: 129, SEQ ID NO: 69, SEQ ID NO: 130, respectively;
s. SEQ ID NO: 132, SEQ ID NO: 133, SEQ ID NO: 134, respectively;
t. SEQ ID NO: 126, SEQ ID NO: 69, SEQ ID NO: 137, respectively;
u. SEQ ID NO: 132, SEQ ID NO: 139, SEQ ID NO: 140, respectively;
v. SEQ ID NO: 122, SEQ ID NO: 69, SEQ ID NO: 123, respectively;
w. SEQ ID NO: 90, SEQ ID NO: 91, SEQ ID NO: 145, respectively;
x. SEQ ID NO: 148, SEQ ID NO: 69, SEQ ID NO: 109, respectively;
y. SEQ ID NO: 149, SEQ ID NO: 63, SEQ ID NO: 109, respectively;
z. SEQ ID NO: 122, SEQ ID NO: 69, SEQ ID NO: 123, respectively;
aa. SEQ ID NO: 126, SEQ ID NO: 69, SEQ ID NO: 130, respectively;
bb. SEQ ID NO: 155, SEQ ID NO: 156, SEQ ID NO: 157, respectively; or
cc. SEQ ID NO: 155, SEQ ID NO: 156, SEQ ID NO: 159, respectively.

13. The antibody of any one of claims 10-12, wherein the light chain variable region comprises four framework regions, which are IFR1, IFR2, IFR3, and IFR4 from N terminus to C terminus, wherein the IFR1 is selected from SEQ ID NOs: 164, 171, 179, 185, 191, 197, 201, 207, 213, 218, 222, 228, 232, 247, 256, 263, 270, 277, 283, 299, and 303; the 1FR2 is selected from SEQ ID NOs: 165, 172, 180, 186, 192, 198, 202, 208, 214, 219, 223, 229, 235, 240, 243, 248, 257, 264, 278, 300, and 304; the IFR3 is selected from SEQ ID NOs: 166, 173, 181, 187, 193, 199, 203, 209, 220, 230, 236, 238, 241, 244, 249, 253, 258, 273, 279, and 301; and the 1FR4 is selected from SEQ ID NOs: 167, 174, 182, 215, 250, 280, and 302.

14. The antibody of claim 13, wherein the 1FR1, 1FR2, 1FR3, and IFR4 comprise:
a. SEQ ID NO: 164, SEQ ID NO: 165, SEQ ID NO: 166, SEQ ID NO: 167, respectively;
b. SEQ ID NO: 171, SEQ ID NO: 172, SEQ ID NO: 173, SEQ ID NO: 174, respectively;
c. SEQ ID NO: 179, SEQ ID NO: 180, SEQ ID NO: 181, SEQ ID NO: 182, respectively;
d. SEQ ID NO: 185, SEQ ID NO: 186, SEQ ID NO: 187, SEQ ID NO: 174, respectively;
e. SEQ ID NO: 191, SEQ ID NO: 192, SEQ ID NO: 193, SEQ ID NO: 174, respectively;
f. SEQ ID NO: 197, SEQ ID NO: 198, SEQ ID NO: 199, SEQ ID NO: 174, respectively;
g. SEQ ID NO: 201, SEQ ID NO: 202, SEQ ID NO: 203, SEQ ID NO: 167, respectively;
h. SEQ ID NO: 207, SEQ ID NO: 208, SEQ ID NO: 209, SEQ ID NO: 174, respectively;
i. SEQ ID NO: 213, SEQ ID NO: 214, SEQ ID NO: 203, SEQ ID NO: 215, respectively;
j. SEQ ID NO: 218, SEQ ID NO: 219, SEQ ID NO: 220, SEQ ID NO: 174, respectively;
k. SEQ ID NO: 222, SEQ ID NO: 223, SEQ ID NO: 209, SEQ ID NO: 174, respectively;
l. SEQ ID NO: 228, SEQ ID NO: 229, SEQ ID NO: 230, SEQ ID NO: 174, respectively;
m. SEQ ID NO: 232, SEQ ID NO: 219, SEQ ID NO: 220, SEQ ID NO: 167, respectively;
n. SEQ ID NO: 201, SEQ ID NO: 235, SEQ ID NO: 236, SEQ ID NO: 174, respectively;
o. SEQ ID NO: 201, SEQ ID NO: 214, SEQ ID NO: 238, SEQ ID NO: 215, respectively;
p. SEQ ID NO: 171, SEQ ID NO: 240, SEQ ID NO: 241, SEQ ID NO: 174, respectively;
q. SEQ ID NO: 171, SEQ ID NO: 243, SEQ ID NO: 244, SEQ ID NO: 174, respectively;
r. SEQ ID NO: 222, SEQ ID NO: 165, SEQ ID NO: 209, SEQ ID NO: 174, respectively;
s. SEQ ID NO: 247, SEQ ID NO: 248, SEQ ID NO: 249, SEQ ID NO: 250, respectively;
t. SEQ ID NO: 171, SEQ ID NO: 243, SEQ ID NO: 253, SEQ ID NO: 167, respectively;
u. SEQ ID NO: 256, SEQ ID NO: 257, SEQ ID NO: 258, SEQ ID NO: 174, respectively;
v. SEQ ID NO: 171, SEQ ID NO: 240, SEQ ID NO: 241, SEQ ID NO: 174, respectively;
w. SEQ ID NO: 263, SEQ ID NO: 264, SEQ ID NO: 199, SEQ ID NO: 174, respectively;
x. SEQ ID NO: 222, SEQ ID NO: 165, SEQ ID NO: 209, SEQ ID NO: 174, respectively;
y. SEQ ID NO: 270, SEQ ID NO: 165, SEQ ID NO: 166, SEQ ID NO: 174, respectively;
z. SEQ ID NO: 171, SEQ ID NO: 240, SEQ ID NO: 241, SEQ ID NO: 174, respectively;
aa. SEQ ID NO: 171, SEQ ID NO: 243, SEQ ID NO: 273, SEQ ID NO: 174, respectively;
bb. SEQ ID NO: 277, SEQ ID NO: 278, SEQ ID NO: 279, SEQ ID NO: 280, respectively; or
cc. SEQ ID NO: 283, SEQ ID NO: 278, SEQ ID NO: 279, SEQ ID NO: 174, respectively,
dd. SEQ ID NO: 299, SEQ ID NO: 300, SEQ ID NO: 301, SEQ ID NO: 302, respectively;
ee. SEQ ID NO: 303, SEQ ID NO: 304, SEQ ID NO: 301, SEQ ID NO: 302, respectively;
ff. SEQ ID NO: 299, SEQ ID NO: 304, SEQ ID NO: 301, SEQ ID NO: 302, respectively;

15. The antibody of any one of claims 13and 14, wherein the CDRs and FRs in the light chain variable region are operably connected in the order of IFR1-1CDR1-1FR2-1CDR2-1FR3-1CDR3-IFR4 from N terminus to C terminus.

16. The antibody of any one of claims 10-15, further comprising a light chain constant region (CL), wherein the CL is SEQ ID NO: 311.

17. The antibody of any one of claims 10-16, wherein the light chain variable region comprises an amino acid sequence with at least 80%, 85%, 90%, 95%, 96%, 97%, 98%, 99%, or 100% identity to any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

18. The antibody of claims 10-17, wherein the light chain variable region comprises an amino acid sequence of any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

19. The antibody of any one of claims 1-9, further comprising a light chain variable region, wherein the light chain variable region comprises at least one CDR, wherein the CDR that is farthest from the N terminus is selected from SEQ ID NOs: 64, 70, 76, 82, 87, 92, 98, 100, 102, 107, 109, 115, 117, 123, 127, 130, 134, 137, 140, 145, 157, and 159.

20. The antibody of claim 18, wherein the light chain variable region comprises three CDRs, which are ICDR1, ICDR2, and ICDR3 from N terminus to C terminus, wherein the 1CDR1 is selected from SEQ ID NOs: 62, 68, 74, 80, 86, 90, 96, 99, 105, 108, 113, 122, 126, 129, 132, 148, 149, and 155; the ICDR2 is selected from SEQ ID NOs: 63, 69, 75, 81, 91, 97, 106, 114, 133, 139, and 156; and the ICDR3 is selected from SEQ ID NOs: 64, 70, 76, 82, 87, 92, 98, 100, 102, 107, 109, 115, 117, 123, 127, 130, 134, 137, 140, 145, 157, and 159.

21. The antibody of claim 20, wherein the ICDR1, 1CDR2, and ICDR3 comprise:
a. SEQ ID NO: 62, SEQ ID NO: 63, SEQ ID NO: 64, respectively;
b. SEQ ID NO: 68, SEQ ID NO: 69, SEQ ID NO: 70, respectively;
c. SEQ ID NO: 74, SEQ ID NO: 75, SEQ ID NO: 76, respectively;
d. SEQ ID NO: 80, SEQ ID NO: 81, SEQ ID NO: 82, respectively;
e. SEQ ID NO: 86, SEQ ID NO: 69, SEQ ID NO: 87, respectively;
f. SEQ ID NO: 90, SEQ ID NO: 91, SEQ ID NO: 92, respectively;
g. SEQ ID NO: 96, SEQ ID NO: 97, SEQ ID NO: 98, respectively;
h. SEQ ID NO: 99, SEQ ID NO: 69, SEQ ID NO: 100, respectively;
i. SEQ ID NO: 96, SEQ ID NO: 97, SEQ ID NO: 102, respectively;
j. SEQ ID NO: 105, SEQ ID NO: 106, SEQ ID NO: 107, respectively;
k. SEQ ID NO: 108, SEQ ID NO: 69, SEQ ID NO: 109, respectively;
l. SEQ ID NO: 113, SEQ ID NO: 114, SEQ ID NO: 115, respectively;
m. SEQ ID NO: 105, SEQ ID NO: 106, SEQ ID NO: 117, respectively;
n. SEQ ID NO: 96, SEQ ID NO: 97, SEQ ID NO: 102, respectively;
o. SEQ ID NO: 96, SEQ ID NO: 97, SEQ ID NO: 102, respectively;
p. SEQ ID NO: 122, SEQ ID NO: 69, SEQ ID NO: 123, respectively;
q. SEQ ID NO: 126, SEQ ID NO: 69, SEQ ID NO: 127, respectively;
r. SEQ ID NO: 129, SEQ ID NO: 69, SEQ ID NO: 130, respectively;
s. SEQ ID NO: 132, SEQ ID NO: 133, SEQ ID NO: 134, respectively;
t. SEQ ID NO: 126, SEQ ID NO: 69, SEQ ID NO: 137, respectively;
u. SEQ ID NO: 132, SEQ ID NO: 139, SEQ ID NO: 140, respectively;
v. SEQ ID NO: 122, SEQ ID NO: 69, SEQ ID NO: 123, respectively;
w. SEQ ID NO: 90, SEQ ID NO: 91, SEQ ID NO: 145, respectively;
x. SEQ ID NO: 148, SEQ ID NO: 69, SEQ ID NO: 109, respectively;
y. SEQ ID NO: 149, SEQ ID NO: 63, SEQ ID NO: 109, respectively;
z. SEQ ID NO: 122, SEQ ID NO: 69, SEQ ID NO: 123, respectively;
aa. SEQ ID NO: 126, SEQ ID NO: 69, SEQ ID NO: 130, respectively;
bb. SEQ ID NO: 155, SEQ ID NO: 156, SEQ ID NO: 157, respectively; or
cc. SEQ ID NO: 155, SEQ ID NO: 156, SEQ ID NO: 159, respectively.

22. The antibody of any one of claims 19-21, wherein the light chain variable region comprises four framework regions, which are IFR1, 1FR2, IFR3, and IFR4 from N terminus to C terminus, wherein the 1FR1 is selected from SEQ ID NOs: 164, 171, 179, 185, 191, 197, 201, 207, 213, 218, 222, 228, 232, 247, 256, 263, 270, 277, 283, 299, and 303; the 1FR2 is selected from SEQ ID NOs: 165, 172, 180, 186, 192, 198, 202, 208, 214, 219, 223, 229, 235, 240, 243, 248, 257, 264, 278, 300, and 304; the IFR3 is selected from SEQ ID NOs: 166, 173, 181, 187, 193, 199, 203, 209, 220, 230, 236, 238, 241, 244, 249, 253, 258, 273, 279, and 301; and the IFR4 is selected from SEQ ID NOs: 167, 174, 182, 215, 250, 280, and 302.

23. The antibody of claim 22, wherein the IFR1, IFR2, IFR3, and IFR4 comprise:
a. SEQ ID NO: 164, SEQ ID NO: 165, SEQ ID NO: 166, SEQ ID NO: 167, respectively;
b. SEQ ID NO: 171, SEQ ID NO: 172, SEQ ID NO: 173, SEQ ID NO: 174, respectively;
c. SEQ ID NO: 179, SEQ ID NO: 180, SEQ ID NO: 181, SEQ ID NO: 182, respectively;
d. SEQ ID NO: 185, SEQ ID NO: 186, SEQ ID NO: 187, SEQ ID NO: 174, respectively;
e. SEQ ID NO: 191, SEQ ID NO: 192, SEQ ID NO: 193, SEQ ID NO: 174, respectively;
f. SEQ ID NO: 197, SEQ ID NO: 198, SEQ ID NO: 199, SEQ ID NO: 174, respectively;
g. SEQ ID NO: 201, SEQ ID NO: 202, SEQ ID NO: 203, SEQ ID NO: 167, respectively;
h. SEQ ID NO: 207, SEQ ID NO: 208, SEQ ID NO: 209, SEQ ID NO: 174, respectively;
i. SEQ ID NO: 213, SEQ ID NO: 214, SEQ ID NO: 203, SEQ ID NO: 215, respectively;
j. SEQ ID NO: 218, SEQ ID NO: 219, SEQ ID NO: 220, SEQ ID NO: 174, respectively;
k. SEQ ID NO: 222, SEQ ID NO: 223, SEQ ID NO: 209, SEQ ID NO: 174, respectively;
l. SEQ ID NO: 228, SEQ ID NO: 229, SEQ ID NO: 230, SEQ ID NO: 174, respectively;
m. SEQ ID NO: 232, SEQ ID NO: 219, SEQ ID NO: 220, SEQ ID NO: 167, respectively;
n. SEQ ID NO: 201, SEQ ID NO: 235, SEQ ID NO: 236, SEQ ID NO: 174, respectively;
o. SEQ ID NO: 201, SEQ ID NO: 214, SEQ ID NO: 238, SEQ ID NO: 215, respectively;
p. SEQ ID NO: 171, SEQ ID NO: 240, SEQ ID NO: 241, SEQ ID NO: 174, respectively;
q. SEQ ID NO: 171, SEQ ID NO: 243, SEQ ID NO: 244, SEQ ID NO: 174, respectively;
r. SEQ ID NO: 222, SEQ ID NO: 165, SEQ ID NO: 209, SEQ ID NO: 174, respectively;
s. SEQ ID NO: 247, SEQ ID NO: 248, SEQ ID NO: 249, SEQ ID NO: 250, respectively;
t. SEQ ID NO: 171, SEQ ID NO: 243, SEQ ID NO: 253, SEQ ID NO: 167, respectively;
u. SEQ ID NO: 256, SEQ ID NO: 257, SEQ ID NO: 258, SEQ ID NO: 174, respectively;
v. SEQ ID NO: 171, SEQ ID NO: 240, SEQ ID NO: 241, SEQ ID NO: 174, respectively;
w. SEQ ID NO: 263, SEQ ID NO: 264, SEQ ID NO: 199, SEQ ID NO: 174, respectively;
x. SEQ ID NO: 222, SEQ ID NO: 165, SEQ ID NO: 209, SEQ ID NO: 174, respectively;
y. SEQ ID NO: 270, SEQ ID NO: 165, SEQ ID NO: 166, SEQ ID NO: 174, respectively;
z. SEQ ID NO: 171, SEQ ID NO: 240, SEQ ID NO: 241, SEQ ID NO: 174, respectively;
aa. SEQ ID NO: 171, SEQ ID NO: 243, SEQ ID NO: 273, SEQ ID NO: 174, respectively;
bb. SEQ ID NO: 277, SEQ ID NO: 278, SEQ ID NO: 279, SEQ ID NO: 280, respectively; or
cc. SEQ ID NO: 283, SEQ ID NO: 278, SEQ ID NO: 279, SEQ ID NO: 174, respectively,
dd. SEQ ID NO: 299, SEQ ID NO: 300, SEQ ID NO: 301, SEQ ID NO: 302, respectively;
ee. SEQ ID NO: 303, SEQ ID NO: 304, SEQ ID NO: 301, SEQ ID NO: 302, respectively;
ff. SEQ ID NO: 299, SEQ ID NO: 304, SEQ ID NO: 301, SEQ ID NO: 302, respectively;

24. The antibody of any one of claims 20 and 21, wherein the CDRs and FRs in the light chain variable region are operably connected in the order of IFR1-ICDR1-IFR2-ICDR2-1FR3-1CDR3-1FR4 from N terminus to C terminus.

25. The antibody of any one of claims 19-24, further comprising a light chain constant region (CL), wherein the CL is SEQ ID NO: 311.

26. The antibody of any one of claims 19-25, wherein the light chain variable region comprises an amino acid sequence with at least 80%, 85%, 90%, 95%, 96%, 97%, 98%, 99%, or 100% identity to any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

27. The antibody of any one of claims 19-26, wherein the light chain variable region comprises an amino acid sequence of any one of SEQ ID NOs: 2, 4, 6, 8, 10, 12, 14, 16, 18, 20, 22, 24, 26, 28, 30, 32, 34, 36, 38, 40, 42, 46, 48, 50, 54, 56, 58, 286, 288, and 289.

28. The antibody of any one of claims 19-27, wherein the heavy chain variable region and the light chain variable region comprise an amino acid sequence with at least 80%, 85%, 90%, 95%, 96%, 97%, 98%, 99%, or 100% identity to:
a. SEQ ID NO: 1, and SEQ ID NO: 2, respectively;
b. SEQ ID NO: 3, and SEQ ID NO: 4, respectively;
c. SEQ ID NO: 5, and SEQ ID NO: 6, respectively;
d. SEQ ID NO: 7, and SEQ ID NO: 8, respectively;
e. SEQ ID NO: 9, and SEQ ID NO: 10, respectively;
f. SEQ ID NO: 11, and SEQ ID NO: 12, respectively;
g. SEQ ID NO: 13, and SEQ ID NO: 14, respectively;
h. SEQ ID NO: 15, and SEQ ID NO: 16, respectively;
i. SEQ ID NO: 17, and SEQ ID NO: 18, respectively;
j. SEQ ID NO: 19, and SEQ ID NO: 20, respectively;
k. SEQ ID NO: 21, and SEQ ID NO: 22, respectively;
l. SEQ ID NO: 23, and SEQ ID NO: 24, respectively;
m. SEQ ID NO: 25, and SEQ ID NO: 26, respectively;
n. SEQ ID NO: 27, and SEQ ID NO: 28, respectively;
o. SEQ ID NO: 29, and SEQ ID NO: 30, respectively;
p. SEQ ID NO: 31, and SEQ ID NO: 32, respectively;
q. SEQ ID NO: 33, and SEQ ID NO: 34, respectively;
r. SEQ ID NO: 35, and SEQ ID NO: 36, respectively;
s. SEQ ID NO: 37, and SEQ ID NO: 38, respectively;
t. SEQ ID NO: 39, and SEQ ID NO: 40, respectively;
u. SEQ ID NO: 41, and SEQ ID NO: 42, respectively;
v. SEQ ID NO: 43, and SEQ ID NO: 32, respectively;
w. SEQ ID NO: 45, and SEQ ID NO: 46, respectively;
x. SEQ ID NO: 47, and SEQ ID NO: 48, respectively;
y. SEQ ID NO: 49, and SEQ ID NO: 50, respectively;
z. SEQ ID NO: 51, and SEQ ID NO: 32, respectively;
aa. SEQ ID NO: 53, and SEQ ID NO: 54, respectively;
bb. SEQ ID NO: 55, and SEQ ID NO: 56, respectively; or
cc. SEQ ID NO: 57, and SEQ ID NO: 58, respectively,
dd. SEQ ID NO: 285, and SEQ ID NO: 286, respectively;
ee. SEQ ID NO: 287, and SEQ ID NO: 288, respectively;
ff. SEQ ID NO: 287, and SEQ ID NO: 289, respectively;
gg. SEQ ID NO: 290, and SEQ ID NO: 288, respectively;
hh. SEQ ID NO: 290, and SEQ ID NO: 286, respectively;
ii. SEQ ID NO: 291, and SEQ ID NO: 286, respectively;
jj. SEQ ID NO: 292, and SEQ ID NO: 293, respectively;
kk. SEQ ID NO: 294, and SEQ ID NO: 293, respectively.

29. The antibody of any one of claims 19-28, wherein the heavy chain variable region and the light chain variable region comprise:
a. SEQ ID NO: 1, and SEQ ID NO: 2, respectively;
b. SEQ ID NO: 3, and SEQ ID NO: 4, respectively;
c. SEQ ID NO: 5, and SEQ ID NO: 6, respectively;
d. SEQ ID NO: 7, and SEQ ID NO: 8, respectively;
e. SEQ ID NO: 9, and SEQ ID NO: 10, respectively;
f. SEQ ID NO: 11, and SEQ ID NO: 12, respectively;
g. SEQ ID NO: 13, and SEQ ID NO: 14, respectively;
h. SEQ ID NO: 15, and SEQ ID NO: 16, respectively;
i. SEQ ID NO: 17, and SEQ ID NO: 18, respectively;
j. SEQ ID NO: 19, and SEQ ID NO: 20, respectively;
k. SEQ ID NO: 21, and SEQ ID NO: 22, respectively;
l. SEQ ID NO: 23, and SEQ ID NO: 24, respectively;
m. SEQ ID NO: 25, and SEQ ID NO: 26, respectively;
n. SEQ ID NO: 27, and SEQ ID NO: 28, respectively;
o. SEQ ID NO: 29, and SEQ ID NO: 30, respectively;
p. SEQ ID NO: 31, and SEQ ID NO: 32, respectively;
q. SEQ ID NO: 33, and SEQ ID NO: 34, respectively;
r. SEQ ID NO: 35, and SEQ ID NO: 36, respectively;
S. SEQ ID NO: 37, and SEQ ID NO: 38, respectively;
t. SEQ ID NO: 39, and SEQ ID NO: 40, respectively;
u. SEQ ID NO: 41, and SEQ ID NO: 42, respectively;
v. SEQ ID NO: 43, and SEQ ID NO: 32, respectively;
w. SEQ ID NO: 45, and SEQ ID NO: 46, respectively;
x. SEQ ID NO: 47, and SEQ ID NO: 48, respectively;
y. SEQ ID NO: 49, and SEQ ID NO: 50, respectively;
z. SEQ ID NO: 51, and SEQ ID NO: 32, respectively;
aa. SEQ ID NO: 53, and SEQ ID NO: 54, respectively;
bb. SEQ ID NO: 55, and SEQ ID NO: 56, respectively; or
cc. SEQ ID NO: 57, and SEQ ID NO: 58, respectively,
dd. SEQ ID NO: 285, and SEQ ID NO: 286, respectively;
ee. SEQ ID NO: 287, and SEQ ID NO: 288, respectively;
ff. SEQ ID NO: 287, and SEQ ID NO: 289, respectively;
gg. SEQ ID NO: 290, and SEQ ID NO: 288, respectively;
hh. SEQ ID NO: 290, and SEQ ID NO: 286, respectively;
ii. SEQ ID NO: 291, and SEQ ID NO: 286, respectively;
jj. SEQ ID NO: 292, and SEQ ID NO: 293, respectively;
kk. SEQ ID NO: 294, and SEQ ID NO: 293, respectively.

30. The antibody of any one of claims 1-29, wherein the antibody specifically binds fibroblast growth factor receptor 2b (FGFR2b).

31. The antibody of claim 30, wherein the FGFR2b is from an origin selected from human, mouse, and cynomolgus monkey.

32. The antibody of any one of claims 1-31, wherein the antibody binds FGFR2b with a K_{D} of about 10 nM or lower.

33. The antibody of any one of claims 1-32, wherein the antibody binds FGFR2b with a K_{D} of about 9 nM or lower.

34. The antibody of any one of claims 1-33, wherein the antibody binds FGFR2b with a K_{D} of about 5 nM or lower.

35. The antibody of any one of claims 1-34, wherein the antibody does not detectably bind to FGFR1b, FGFR1c, FGFR2c, FGFR3b, FGFR3c, or FGFR4.

36. The antibody of any one of claims 1-35, wherein the antibody is a chimeric antibody.

37. The antibody of any one of claims 1-36, wherein the antibody is a humanized or partially humanized antibody.

38. The antibody of any one of claims 1-37, wherein the antibody is a monoclonal antibody.

39. The antibody of any one of claims 1-38, wherein the antibody is a bispecific antibody.

40. The antibody of any one of claims 1-39, wherein the antibody is operably linked to a cytotoxic agent.

41. The antibody of any one of claims 1-40, wherein the antibody is an afucosylated antibody.

42. The antibody of any one of claims 1-41, wherein the antibody comprises a Fc region, and the Fc region is engineered to enhance antibody-dependent cellular cytotoxicity (ADCC).

43. The antibody of any one of claims 1-42, wherein the antibody is an ADCC-enhanced antibody.

44. A composition, comprising the antibody of any one of claims 1-43.

45. A pharmaceutical composition, comprising the antibody of any one of claims 1-43 and a pharmaceutically acceptable carrier.

46. A polynucleotide encoding the antibody of any one of claims 1-43.

47. A vector, comprising the polynucleotide of claim 46.

48. A cell capable of expressing the antibody of any one of claims 1-43.

49. A cell comprising the polynucleotide of claim 40 and/or the vector of claim 47.

50. A method of producing an antibody, comprising culturing the cell of claim 48 or 49 and recovering the antibody from the cell.

51. A method for blocking FGFR2b from binding to at least one fibroblast growth factor (FGF), comprising administering to a subject an effective amount of the antibody of any one of claims 1-43 or the composition of claim 44 or 45.

52. The method of claim 51, wherein the FGF is selected from FGF1, FGF3, FGF7, FGF10, and FGF22.

53. A method for inhibiting cell proliferation, comprising administering to a subject an effective amount of the antibody of any one of claims 1-43 or the composition of claim 44 or 45.

54. The method of claim 53, wherein the cell proliferation is FGF-induced cell proliferation.

55. The method of claim 53 or 54, wherein the cell is a cancer cell.

56. A method for inhibiting the signal transduction pathway stimulated by binding of FGF to FGFR2b, comprising administering to a subject an effective amount of the antibody of any one of claims 1-43 or the composition of claim 44 or 45.

57. A method for inhibiting the growth of a tumor xenograft that overexpresses FGFR2b, comprising administering to a subject an effective amount of the antibody of any one of claims 1-43 or the composition of claim 44 or 45.

58. A method for detecting the presence of FGFR2b in a sample, comprising contacting the antibody of any one of claims 1-43 or the composition of claim 44 or 45 with the sample, wherein a detection of at least one bound antibody indicates presence of FGFR2b.


SEQUENCE LISTING (313 sequences)

SEQ ID NO: 1 - protein, 113 aa, synthetic construct
  REGION 1..113: note = mAb1-VH
QAYLQQSGAELVRPGASVKMSCKASGYTFTSYNMHWVKQTPRQGLEWIGSIYPENGDTSYNQKFRDKATLTVDKSSSTAYMQLSSLTSEDSAVYFCARPFDYWGQGTTLTVSS

SEQ ID NO: 2 - protein, 107 aa, synthetic construct
  REGION 1..107: note = mAb1-VL
SIVMTQTPKFLPISAGDRVTMTCKASQSVGNNVAWYQQKPGQSPKLLIYYASNRYTGVPDRFTGSGSGTDFTFTISSVQVEDLAVYFCQQFYSSPYTFGSGTKLEIK

SEQ ID NO: 3 - protein, 114 aa, synthetic construct
  REGION 1..114: note = mAb2-VH
EVQLQQSGTVLARPGASVKMSCRASGYTFTDYWMHWVKQRPGQGLEWIGAIYPGNTDTDYNQKFKGKAKLTAVTSASSAYMELSSLTNEDSAVYYCTGFNLDYWGQGTTLTVSS

SEQ ID NO: 4 - protein, 107 aa, synthetic construct
  REGION 1..107: note = mAb2-VL
DIVMTQSQKFMSTSVGDRVSVTCKASQNVGTSVAWYQKKPGQSPKVVIYSASTRYTGVPDRFTGSGLGTDFTLTISNVQSEDLAEYFCQQYGSSPYTFGGGTKLEIK

SEQ ID NO: 5 - protein, 115 aa, synthetic construct
  REGION 1..115: note = mAb3-VH
DVQLQESGPGLVKPSQSLSLTCSVTGDSITSGYYWNWIRQFPGNKLEWMGYISYEGSNNYNPSLKNRISITRDTSKNQFFLRLNSVTTEDTATYYCARDLDMDYWGQGTSVTVSS

SEQ ID NO: 6 - protein, 107 aa, synthetic construct
  REGION 1..107: note = mAb3-VL
DIQMTQTTSSLSASLGDRVTISCRASQDINNYLNWYQQKPDGTVKLLIYYISRLHSGVPLRFSGSGSGTDFSLTISNLEPEDIATYYCQQYSELPLTFGGGTKLELK

SEQ ID NO: 7 - protein, 114 aa, synthetic construct
  REGION 1..114: note = mAb4-VH
QAYLQQSGAELVRPGASVKMSCKASGYTFTNYNMHWVKQTPRQGLEWIGAIYPGNGDTSYNQKFKGKATLTVDKSSSTAYMQLSSLTSEDSAVYFCARGAMDYWGQGTSVTVSS

SEQ ID NO: 8 - protein, 107 aa, synthetic construct
  REGION 1..107: note = mAb4-VL
EIQMTQSPSSMSASLGDRITITCQATQDIVKNLNWYQQKPGKPPSFLIYYATELAEGVPSRFSGSGSGSDYSLTISNLESEDFADYYCLQSYESPYTFGGGTKLEIK

SEQ ID NO: 9 - protein, 113 aa, synthetic construct
  REGION 1..113: note = mAb5-VH
EVQLQQSGTVLARPGASVKMSCKTSGYTFTNYWMHWVKQRPGQGLEWIGAIYPENSDTSYNQKFKDKAKVTAVTSASTAYMEVSSLTNEDSAVYYCTWGLAYWGQGTLVTVSA

SEQ ID NO: 10 - protein, 107 aa, synthetic construct
  REGION 1..107: note = mAb5-VL
DIVMTQSQKFMSTLVGDRVSVTCKASQNVGTDVAWFQKKPGQSPKALIYSASTRYTGVPDHFTGSGSGTDFTLTISNVQSEDLAEYFCHQYSSSPYTFGGGTKLEIK

SEQ ID NO: 11 - protein, 116 aa, synthetic construct
  REGION 1..116: note = mAb6-VH
EVQLQQSGTVLARPGASVKMSCKASGYTFTSYWMHWVKQRPGQGLEWIGAIYPENSDTNYNQKFKGKAKLTAVTSASTAYMDLSSLTNEDSAVYYCANYYGSSDSWGQGTTLTVSA

SEQ ID NO: 12 - protein, 106 aa, synthetic construct
  REGION 1..106: note = mAb6-VL
ENVLTQSPAIMSATLGEKVTMSCRASSNVKYMYWSQQKSGASPKLWIYYTSNLASGVPARFSGSGSGTSYSLTISSVEAEDAATYYCQQFTSSPYTFGGGTKLEIK

SEQ ID NO: 13 - protein, 118 aa, synthetic construct
  REGION 1..118: note = mAb7-VH
EVQLQQSGTVLARPGASVKMSCKASGYSFTSYWMHWVKQRPGQGLEWIGAIYPGNSDTSYNQKFKGKAKLTAVTSASTAYMELSSLTNEDSAVYYCTTFYDYDVIDYWGQGTTLTVSS

SEQ ID NO: 14 - protein, 106 aa, synthetic construct
  REGION 1..106: note = mAb7-VL
QIVLTQSPAIMSASPGEKVTITCSASSSVSYMHWLQQKPGTSPKLWIYSTSNLASGVPARFSGSGSGTSYSLTISRMEAEDAATYYCQERSSSPFTFGSGTKLEIK

SEQ ID NO: 15 - protein, 114 aa, synthetic construct
  REGION 1..114: note = mAb8-VH
QAYLQQSGAELVRSGASVKMSCKASGYTFTSYNMHWVKQTPGQGLEWIGYIYPGNGDTNYNQKFKGKATLTADTSSSTAYMQISSLTSEDSAVYFCARGAMDYWGQGTSVTVSS

SEQ ID NO: 16 - protein, 107 aa, synthetic construct
  REGION 1..107: note = mAb8-VL
DIVMTQSHKFMSTSVGDRVNITCKASQDVSIAVAWYQHKPGQSPKLLIYSASYRYTGVPDRFTGSGSGTDFTFTISSVQAEDLAVYYCQQHYSSPYSFGGGTKLEIK

SEQ ID NO: 17 - protein, 117 aa, synthetic construct
  REGION 1..117: note = mAb9-VH
EVQLQQSGTLLARPGASVKMSCKASGYSFTSYWMHWVKQRPGQGLEWIGAIYPGNSDTTYNQKFKGKARLTAVTSASTAYMELSSLTNEDSAVYYCTIYYDYDVDYWGQGTSVSVSS

SEQ ID NO: 18 - protein, 106 aa, synthetic construct
  REGION 1..106: note = mAb9-VL
QIVLTQSPAIMSASPGEKVTMTCSASSSVSYMYWLQQKPGTSPKLWIYSTSNLASGVPARFSGSGSGTSYSLTISRMEAEDAATYYCQQRSSYPYTFGGGANLEIK

SEQ ID NO: 19 - protein, 112 aa, synthetic construct
  REGION 1..112: note = mAb10-VH
EVQLQQSGTVLARPGASVKMSCKASDYTFTSYWMHWVKQRPGQGLEWIGAIYPGNSDTSYNQKFKDKAKLTAVTSTSTAYMELSSLTNEDSAVYYCSHFDVWGAGTTVTVSS

SEQ ID NO: 20 - protein, 106 aa, synthetic construct
  REGION 1..106: note = mAb10-VL
QIVLTQSPALMSASPGEKVTMTCSASSSVNYMYWYQQKPRSSPKPWIYLTSNLASGVPARFSGSGSGTSYSLTISSMEAEDAATYYCQQWSSNPYTFGGGTKLEIK

SEQ ID NO: 21 - protein, 114 aa, synthetic construct
  REGION 1..114: note = mAb11-VH
QAYLQQSGAELVRSGASVKMSCKASGYTFTSYNMHWVKRTPGQGLEWIGYIYPGNGDTNYNQKFKGKATLTADTSSSTAYMQISSLTSEDSAVYFCARGAMDYWGQGTSVTVSS

SEQ ID NO: 22 - protein, 107 aa, synthetic construct
  REGION 1..107: note = mAb11-VL
DIVMTQSHKFMSTSVGDRVSITCKASQDVSSNVAWYQQSPGQSPKLLIYSASYRYTGVPDRFTGSGSGTDFTFTISSVQAEDLAVYYCQQHYSSPYTFGGGTKLEIK

SEQ ID NO: 23 - protein, 122 aa, synthetic construct
  REGION 1..122: note = mAb12-VH
QVQLQQPGAELVKPGASVKMSCKASGYSFNSYWMHWVKQRPGQGLEWIGVTDPSGSYRSYNQKFKGKATLTVDTSSSTAYMQLSSLTSEDSAVYYCTRGDYYGTSPYYFDYWGQGTTLTVPS

SEQ ID NO: 24 - protein, 111 aa, synthetic construct
  REGION 1..111: note = mAb12-VL
DIVLTQSPASLAVSLGQRATISCRASESVDDYGISFMNWFQQKPGQPPKLLIYAASNQGSGVPARFSGSGSGTDFSLNIHPMEEDDTAMYFCQQSKEVPWTFGGGTKLEIK

SEQ ID NO: 25 - protein, 112 aa, synthetic construct
  REGION 1..112: note = mAb13-VH
EVQLQQSGTVLARPGASVKMSCKASGYTFTSYWMHWVKQRPGQGLEWIGAIYPENSDTDYNQKFKGKAKLTAVTSTSTAYMELSSLTNEDSAVYYCSYVDYWGQGTTLTVSS

SEQ ID NO: 26 - protein, 106 aa, synthetic construct
  REGION 1..106: note = mAb13-VL
QIVLTQSPAVMSASPGEKVTMTCSASSSVNYMYWYQQKPRSSPKPWIYLTSNLASGVPARFSGSGSGTSYSLTISSMEAEDAATYYCQQWSSNPFTFGSGTKLEIK

SEQ ID NO: 27 - protein, 117 aa, synthetic construct
  REGION 1..117: note = mAb14-VH
EVHLQQSGTVLARPGASVKMSCKASGYSFTSYWMHWVKQRPGQGLEWIGAIYPGNSDTSYNQKFKAKAKLTAVTSASTAYMELSSLTNEDSAVYYCTIYYDYDVDYWGQGTSVTVSS

SEQ ID NO: 28 - protein, 106 aa, synthetic construct
  REGION 1..106: note = mAb14-VL
QIVLTQSPAIMSASPGEKVTITCSASSSVSYMYWVQQKPGTSPKLWIYSTSKLASGVPARFSGSGSGTSYSLTISRMEAEDAATYYCQQRSSYPYTFGGGTKLEIK

SEQ ID NO: 29 - protein, 117 aa, synthetic construct
  REGION 1..117: note = mAb15-VH
EVQLQQSGTLLARPGASVKMSCKASGYSFTSYWMHWVKQRPGQNLEWIGAIYPGNRDTTYNQKFKGKARLTAVTSASTAYMELSSLTNEDSAVYYCTIYYDYDVDYWGQGTSVSVSS

SEQ ID NO: 30 - protein, 106 aa, synthetic construct
  REGION 1..106: note = mAb15-VL
QIVLTQSPAIMSASPGEKVTITCSASSSVSYMYWLQQKPGTSPKLWIYSTSNLASGVPDRFSGSGSGTSYSLTISRMEAEDAATYYCQQRSSYPYTFGGGANLEIK

SEQ ID NO: 31 - protein, 113 aa, synthetic construct
  REGION 1..113: note = mAb16-VH
EVQLQQSGTVLARPGASVKMSCKASGSTFTNYWMHWVKQRPGQGLEWIGAIYPENGDISYNQKFKGKAKLTAVTSASTAYMDLSSLTNEDSAVYYCTREIAYWGQGTLVTVSA

SEQ ID NO: 32 - protein, 107 aa, synthetic construct
  REGION 1..107: note = mAb16-VL
DIVMTQSQKFMSTSVGDRVSVTCKASQNVGTAVAWYQKKPGQSPKALIYSASTRYTGVPDRFTGSGSGTDFTLTISNVQSEDLAEYFCQQYSSSPYTFGGGTKLEIK

SEQ ID NO: 33 - protein, 113 aa, synthetic construct
  REGION 1..113: note = mAb17-VH
EVQLQQSGTVLARPGASVKMSCKASGSTFTNYWMHWVKQRPGQGLEWIGAIYPENNDVDYNQKFKGKAKLTAVTSASTAYMELSSLTNEDSAVYYCTREITYWGQGTSVTVSS

SEQ ID NO: 34 - protein, 107 aa, synthetic construct
  REGION 1..107: note = mAb17-VL
DIVMTQSQKFMSTSVGDRVSVTCKASQNVGTNVAWYQQKPGQSPKALIYSASYRYSGVPDRFTGSGSGTDFTLTISNVQSEDLADYFCQQHYSNPYTFGGGTKLEIK

SEQ ID NO: 35 - protein, 114 aa, synthetic construct
  REGION 1..114: note = mAb18-VH
QVHVKQSGAELVRPGASVKMSCKASGYTFTNYNMHWVKQTPRQGLEWIGAIYPGNGDTSYNQKFKGKATLTVDKSSSTAYMQLSSLTSEDSAVYFCTRGPFDYWGQGTTLTVSS

SEQ ID NO: 36 - protein, 107 aa, synthetic construct
  REGION 1..107: note = mAb18-VL
DIVMTQSHKFMSTSVGDRVSITCKASQDVSTDVAWYQQKPGQSPKLLIYSASYRYTGVPDRFTGSGSGTDFTFTISSVQAEDLAVYYCQQHYSTPYTFGGGTKLEIK

SEQ ID NO: 37 - protein, 113 aa, synthetic construct
  REGION 1..113: note = mAb19-VH
EVQLQQSGTVLARPGASVKMSCKASGYTFTNYWMHWIIQRPGQGLEWIGSIYPGNSDTSYNQKFMDKAKLTAVTSASTAYLELSSLTNEDSAVYYCTKPLDSWGQGTTLTVSS

SEQ ID NO: 38 - protein, 107 aa, synthetic construct
  REGION 1..107: note = mAb19-VL
DIQMTQSPASLSASVGETVTITCRASENIYSNLAWYQQKEGKSPQLLVYSATKLADGVPSRFSGSGSGTQFSLKIKSLQPEDFGSYYCQHFYGAPYTFGGGTKVEIK

SEQ ID NO: 39 - protein, 112 aa, synthetic construct
  REGION 1..112: note = mAb20-VH
EVQLQQSGTVLARPGASVKMSCKTSGYTFTNYYIHWIKQRPGQGLEYIGAIYPENSDTTYNQKFKGKAKLTAVTSASTAYMDLNSLTDEDSAVYYCSKFDFWGQGTTLTVSS

SEQ ID NO: 40 - protein, 107 aa, synthetic construct
  REGION 1..107: note = mAb20-VL
DIVMTQSQKFMSTSVGDRVSVTCKASQNVGTNVAWYQQKPGQSPKALIYSASYRYSGVPARFTGSGSGTDFTLTISNVQSEDLAEYYCQQHYSTPFTFGSGTKLEIK

SEQ ID NO: 41 - protein, 113 aa, synthetic construct
  REGION 1..113: note = mAb21-VH
EVQLQQSGTVLARPGASVKMSCKTSGYTFTSYWMHWVKQRPGQGLEWIGAIYPENSDTNYNQNFKGKAKLTAVTSASTAYMELSSLTNEDSAVYYCTTRLHYWGTGTTVTISS

SEQ ID NO: 42 - protein, 107 aa, synthetic construct
  REGION 1..107: note = mAb21-VL
DIVMTQSPASLSASVGETVTMTCRASENIYSNLAWYQQKQGKSPQLLVYAATNLADGVPSRFSGSGSGTQFSLKINSLQPEDFGSYYCQHFYGTPYTFGGGTKLEIK

SEQ ID NO: 43 - protein, 113 aa, synthetic construct
  REGION 1..113: note = mAb22-VH
EVQLQQSGAELARPGASVKMSCKTSGYTFTNYWMHWVKQRPGQGLEWIGAIYPDNGDTDYNQKFMDKAKLTSVTSASTAYMELSSLTDEDSAVYFCTYAIAYWGQGTLVTVSA

SEQ ID NO: 44 - skipped

SEQ ID NO: 45 - protein, 116 aa, synthetic construct
  REGION 1..116: note = mAb23-VH
EVQLQQSGTVLARPGASVKMSCKASGYTFTIYWMHWVKQRPGQGLEWIGSIYPENSDTNYNQKFKGKAKLTAVTSASTAYLDLSSLTNEDSAVYYCTRYYGDYDDWGQGTTLTVSS

SEQ ID NO: 46 - protein, 105 aa, synthetic construct
  REGION 1..105: note = mAb23-VL
DIVLTQSPAIMSATLGEKVTMSCRASSNVKYMYWYQQKSGASPKLWIYYTSNLASGVPARFSGSGSGTSYSLTISSVEAEDAATYYCQQFTSSPSFGGGTKLEIK

SEQ ID NO: 47 - protein, 113 aa, synthetic construct
  REGION 1..113: note = mAb24-VH
EVQLQQSGAVLARPGTSVKMSCKASGYTFTDYYIHWIKQRPGQGLEWLGAIYPGNSDTDYNQKVRAKAKLTAVTSASTAYMELSSLTNEDSAVYYCTRGGLDWGQGTTLTVSS

SEQ ID NO: 48 - protein, 107 aa, synthetic construct
  REGION 1..107: note = mAb24-VL
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSSPYTFGGGTKLEIK

SEQ ID NO: 49 - protein, 113 aa, synthetic construct
  REGION 1..113: note = mAb25-VH
EVQLQQSGPELVKPGASVKLSCKASGSTFTNYWMHWVKQRPGQGLEWIGAIYPENGDISYNQKFKGKAKLTAVTSASTAYMDLSSLTNEDSAVYYCTREIAYWGQGTLVTVSS

SEQ ID NO: 50 - protein, 107 aa, synthetic construct
  REGION 1..107: note = mAb25-VL
DIVMTQSPKFLPVTAEDRVTITCKASQSVSNEVAWYQQKPGQSPKLLIYYASNRYTGVPDRFTGSGSGTDFTFTISSVQVEDLAVYFCQQHYSSPYTFGGGTKLEIK

SEQ ID NO: 51 - protein, 113 aa, synthetic construct
  REGION 1..113: note = mAb26-VH
EVQLQQSGTVLARPGASVKMSCKASGSTFTNYWMHWVKQRPGQGLEWIGAIYPENNDVSYNQKFKGKAKLTAVTSASTAYMELSSLTNEDSAVYYCTREITYWGQGTLVTVSA

SEQ ID NO: 52 - skipped

SEQ ID NO: 53 - protein, 112 aa, synthetic construct
  REGION 1..112: note = mAb27-VH
EVQLQQSGPELVKPGASVKISCKASGYTFTTYWMHWVKQRPGQGLEWIGAIYPENSDTSYNQKFKDKAKLTAVTSANTAYMELSSLTNEDSAVYYCTRAFDWGQGTLVTVSA

SEQ ID NO: 54 - protein, 107 aa, synthetic construct
  REGION 1..107: note = mAb27-VL
DIVMTQSQKFMSTSVGDRVSVTCKASQNVGTNVAWYQQKPGQSPKALIYSASYRYSGVPDRFTGSGSGTDFTLTISSVQAEDLAVYYCQQHYSTPYTFGGGTKLEIK

SEQ ID NO: 55 - protein, 119 aa, synthetic construct
  REGION 1..119: note = mAb28-VH
QVQLQQSGSELVKPGASVKLSCKASGYTFTSYDINWVKQRPGQGLEWIGWIYPREIIPKYNEKFKGKATLTVDTSSSTAYMELHSLTSEDSAVYFCAMWGSHLYALDYWGQGTSVTVSS

SEQ ID NO: 56 - protein, 109 aa, synthetic construct
  REGION 1..109: note = mAb28-VL
DIELTQSPALMATSPGEKVTITCSVSSSISSSNLHWYQQKSGTSPKLWIYGTSNLASGVPVRFSGSGSGTSYSLTISSMEAEDAATYYCQQWSSYPHITFGAGTKLELK

SEQ ID NO: 57 - protein, 118 aa, synthetic construct
  REGION 1..118: note = mAb29-VH
QVQLQQSGTVLARPGASVKMSCKASGYTFTSYWMHWLKQRPGQGLEWIGAIYPGNSDTNYNQNFKGKAKLTAVTSASTAYMELSSLTNEDSAVYYCTRGPDGYFEDYWGQGTTLTVSS

SEQ ID NO: 58 - protein, 108 aa, synthetic construct
  REGION 1..108: note = mAb29-VL
DIVLTQSPASLAVSLGQKVTITCSVSSSISSSNLHWYQQKSGTSPKLWIYGTSNLASGVPVRFSGSGSGTSYSLTISSMEAEDAATYYCQQWSSYPYTFGGGTKLEIK

SEQ ID NO: 59 - protein, 8 aa, synthetic construct
  REGION 1..8: note = mAb1-HCDR1, mAb8-HCDR1, mAb11-HCDR1
GYTFTSYN

SEQ ID NO: 60 - protein, 8 aa, synthetic construct
  REGION 1..8: note = mAb1-HCDR2
IYPENGDT

SEQ ID NO: 61 - protein, 6 aa, synthetic construct
  REGION 1..6: note = mAb1-HCDR3
ARPFDY

SEQ ID NO: 62 - protein, 6 aa, synthetic construct
  REGION 1..6: note = mAb1-LCDR1
QSVGNN

SEQ ID NO: 63 - skipped

SEQ ID NO: 64 - protein, 9 aa, synthetic construct
  REGION 1..9: note = mAb1-LCDR3
QQFYSSPYT

SEQ ID NO: 65 - protein, 8 aa, synthetic construct
  REGION 1..8: note = mAb2-HCDR1
GYTFTDYW

SEQ ID NO: 66 - protein, 8 aa, synthetic construct
  REGION 1..8: note = mAb2-HCDR2
IYPGNTDT

SEQ ID NO: 67 - protein, 7 aa, synthetic construct
  REGION 1..7: note = mAb2-HCDR3
TGFNLDY

SEQ ID NO: 68 - protein, 6 aa, synthetic construct
  REGION 1..6: note = mAb2-LCDR1
QNVGTS

SEQ ID NO: 69 - skipped

SEQ ID NO: 70 - protein, 9 aa, synthetic construct
  REGION 1..9: note = mAb2-LCDR3
QQYGSSPYT

SEQ ID NO: 71 - protein, 9 aa, synthetic construct
  REGION 1..9: note = mAb3-HCDR1
GDSITSGYY

SEQ ID NO: 72 - protein, 7 aa, synthetic construct
  REGION 1..7: note = mAb3-HCDR2
ISYEGSN

SEQ ID NO: 73 - protein, 8 aa, synthetic construct
  REGION 1..8: note = mAb3-HCDR3
ARDLDMDY

SEQ ID NO: 74 - protein, 6 aa, synthetic construct
  REGION 1..6: note = mAb3-LCDR1
QDINNY

SEQ ID NO: 75 - skipped

SEQ ID NO: 76 - protein, 9 aa, synthetic construct
  REGION 1..9: note = mAb3-LCDR3
QQYSELPLT

SEQ ID NO: 77 - protein, 8 aa, synthetic construct
  REGION 1..8: note = mAb4-HCDR1, mAb18-HCDR1
GYTFTNYN

SEQ ID NO: 78 - protein, 8 aa, synthetic construct
  REGION 1..8: note = mAb4,8,11,18-HCDR2
IYPGNGDT

SEQ ID NO: 79 - protein, 7 aa, synthetic construct
  REGION 1..7: note = mAb4,8,11-HCDR3
ARGAMDY

SEQ ID NO: 80 - protein, 6 aa, synthetic construct
  REGION 1..6: note = mAb4-LCDR1
QDIVKN

SEQ ID NO: 81 - skipped

SEQ ID NO: 82 - protein, 9 aa, synthetic construct
  REGION 1..9: note = mAb4-LCDR3
LQSYESPYT

SEQ ID NO: 83 - protein, 8 aa, synthetic construct
  REGION 1..8: note = mAb5,19,22-HCDR1
GYTFTNYW

SEQ ID NO: 84 - protein, 8 aa, synthetic construct
  REGION 1..8: note = mAb5,6,13,20,21,23,27-HCDR2
IYPENSDT

SEQ ID NO: 85 - protein, 6 aa, synthetic construct
  REGION 1..6: note = mAb5-HCDR3
TWGLAY

SEQ ID NO: 86 - protein, 6 aa, synthetic construct
  REGION 1..6: note = mAb5-LCDR1
QNVGTD

SEQ ID NO: 87 - protein, 9 aa, synthetic construct
  REGION 1..9: note = mAb5-LCDR3
HQYSSSPYT

SEQ ID NO: 88 - protein, 8 aa, synthetic construct
  REGION 1..8: note = mAb6,13,21,29-HCDR1
GYTFTSYW

SEQ ID NO: 89 - protein, 9 aa, synthetic construct
  REGION 1..9: note = mAb6-HCDR3
ANYYGSSDS

SEQ ID NO: 90 - protein, 5 aa, synthetic construct
  REGION 1..5: note = mAb6,23-LCDR1
SNVKY

SEQ ID NO: 91 - skipped

SEQ ID NO: 92 - protein, 9 aa, synthetic construct
  REGION 1..9: note = mAb6-LCDR3
QQFTSSPYT

SEQ ID NO: 93 - protein, 8 aa, synthetic construct
  REGION 1..8: note = mAb7,9,14,15-HCDR1
GYSFTSYW

SEQ ID NO: 94 - protein, 8 aa, synthetic construct
  REGION 1..8: note = mAb7,9,10,14,19,24,29-HCDR2
IYPGNSDT

SEQ ID NO: 95 - protein, 11 aa, synthetic construct
  REGION 1..11: note = mAb7-HCDR3
TTFYDYDVIDY

SEQ ID NO: 96 - protein, 5 aa, synthetic construct
  REGION 1..5: note = mAb7,9,14,15-LCDR1
SSVSY

SEQ ID NO: 97 - skipped

SEQ ID NO: 98 - protein, 9 aa, synthetic construct
  REGION 1..9: note = mAb7-LCDR3
QERSSSPFT

SEQ ID NO: 99 - protein, 6 aa, synthetic construct
  REGION 1..6: note = mAb8-LCDR1
QDVSIA

SEQ ID NO: 100 - protein, 9 aa, synthetic construct
  REGION 1..9: note = mAb8-LCDR3
QQHYSSPYS

SEQ ID NO: 101 - protein, 10 aa, synthetic construct
  REGION 1..10: note = mAb9,14,15-HCDR3
TIYYDYDVDY

SEQ ID NO: 102 - protein, 9 aa, synthetic construct
  REGION 1..9: note = mAb9,14,15-LCDR3
QQRSSYPYT

SEQ ID NO: 103 - protein, 8 aa, synthetic construct
  REGION 1..8: note = mAb10-HCDR1
DYTFTSYW

SEQ ID NO: 104 - protein, 5 aa, synthetic construct
  REGION 1..5: note = mAb10-HCDR3
SHFDV

SEQ ID NO: 105 - protein, 5 aa, synthetic construct
  REGION 1..5: note = mAb10, 13-LCDR1
SSVNY

SEQ ID NO: 106 - skipped

SEQ ID NO: 107 - protein, 9 aa, synthetic construct
  REGION 1..9: note = mAb10-LCDR3
QQWSSNPYT

SEQ ID NO: 108 - protein, 6 aa, synthetic construct
  REGION 1..6: note = mAb11-LCDR1
QDVSSN

SEQ ID NO: 109 - protein, 9 aa, synthetic construct
  REGION 1..9: note = mAb11,24,25-LCDR3
QQHYSSPYT

SEQ ID NO: 110 - protein, 8 aa, synthetic construct
  REGION 1..8: note = mAb12-HCDR1
GYSFNSYW

SEQ ID NO: 111 - protein, 8 aa, synthetic construct
  REGION 1..8: note = mAb12-HCDR2
TDPSGSYR

SEQ ID NO: 112 - protein, 15 aa, synthetic construct
  REGION 1..15: note = mAb12-HCDR3
TRGDYYGTSPYYFDY

SEQ ID NO: 113 - protein, 10 aa, synthetic construct
  REGION 1..10: note = mAb12-LCDR1
ESVDDYGISF

SEQ ID NO: 114 - skipped

SEQ ID NO: 115 - protein, 9 aa, synthetic construct
  REGION 1..9: note = mAb12-LCDR3
QQSKEVPWT

SEQ ID NO: 116 - protein, 5 aa, synthetic construct
  REGION 1..5: note = mAb13-HCDR3
SYVDY

SEQ ID NO: 117 - protein, 9 aa, synthetic construct
  REGION 1..9: note = mAb13-LCDR3
QQWSSNPFT

SEQ ID NO: 118 - protein, 8 aa, synthetic construct
  REGION 1..8: note = mAb15-HCDR2
IYPGNRDT

SEQ ID NO: 119 - protein, 8 aa, synthetic construct
  REGION 1..8: note = mAb16,17,25,26-HCDR1
GSTFTNYW

SEQ ID NO: 120 - protein, 8 aa, synthetic construct
  REGION 1..8: note = mAb16,25-HCDR2
IYPENGDI

SEQ ID NO: 121 - protein, 6 aa, synthetic construct
  REGION 1..6: note = mAb16-HCDR3, mAb25-HCDR3
TREIAY

SEQ ID NO: 122 - protein, 6 aa, synthetic construct
  REGION 1..6: note = mAb16,22,26-LCDR1
QNVGTA

SEQ ID NO: 123 - protein, 9 aa, synthetic construct
  REGION 1..9: note = mAb16,22,26-LCDR3
QQYSSSPYT

SEQ ID NO: 124 - protein, 8 aa, synthetic construct
  REGION 1..8: note = mAb17, 26-HCDR2
IYPENNDV

SEQ ID NO: 125 - protein, 6 aa, synthetic construct
  REGION 1..6: note = mAb17,26-HCDR3
TREITY

SEQ ID NO: 126 - protein, 6 aa, synthetic construct
  REGION 1..6: note = mAb17,20,27-LCDR1
QNVGTN

SEQ ID NO: 127 - protein, 9 aa, synthetic construct
  REGION 1..9: note = mAb17-LCDR3
QQHYSNPYT

SEQ ID NO: 128 - protein, 7 aa, synthetic construct
  REGION 1..7: note = mAb18-HCDR3
TRGPFDY

SEQ ID NO: 129 - protein, 6 aa, synthetic construct
  REGION 1..6: note = mAb18-LCDR1
QDVSTD

SEQ ID NO: 130 - protein, 9 aa, synthetic construct
  REGION 1..9: note = mAb18,27-LCDR3
QQHYSTPYT

SEQ ID NO: 131 - protein, 6 aa, synthetic construct
  REGION 1..6: note = mAb19-HCDR3
TKPLDS

SEQ ID NO: 132 - protein, 6 aa, synthetic construct
  REGION 1..6: note = mAb19,21-LCDR1
ENIYSN

SEQ ID NO: 133 - skipped

SEQ ID NO: 134 - protein, 9 aa, synthetic construct
  REGION 1..9: note = mAb19-LCDR3
QHFYGAPYT

SEQ ID NO: 135 - protein, 8 aa, synthetic construct
  REGION 1..8: note = mAb20-HCDR1
GYTFTNYY

SEQ ID NO: 136 - protein, 5 aa, synthetic construct
  REGION 1..5: note = mAb20-HCDR3
SKFDF

SEQ ID NO: 137 - protein, 9 aa, synthetic construct
  REGION 1..9: note = mAb20-LCDR3
QQHYSTPFT

SEQ ID NO: 138 - protein, 6 aa, synthetic construct
  REGION 1..6: note = mAb21-HCDR3
TTRLHY

SEQ ID NO: 139 - skipped

SEQ ID NO: 140 - protein, 9 aa, synthetic construct
  REGION 1..9: note = mAb21-LCDR3
QHFYGTPYT

SEQ ID NO: 141 - protein, 8 aa, synthetic construct
  REGION 1..8: note = mAb22-HCDR2
IYPDNGDT

SEQ ID NO: 142 - protein, 6 aa, synthetic construct
  REGION 1..6: note = mAb22-HCDR3
TYAIAY

SEQ ID NO: 143 - protein, 8 aa, synthetic construct
  REGION 1..8: note = mAb23-HCDR1
GYTFTIYW

SEQ ID NO: 144 - protein, 9 aa, synthetic construct
  REGION 1..9: note = mAb23-HCDR3
TRYYGDYDD

SEQ ID NO: 145 - protein, 8 aa, synthetic construct
  REGION 1..8: note = mAb23-LCDR3
QQFTSSPS

SEQ ID NO: 146 - protein, 8 aa, synthetic construct
  REGION 1..8: note = mAb24-HCDR1
GYTFTDYY

SEQ ID NO: 147 - protein, 6 aa, synthetic construct
  REGION 1..6: note = mAb24-HCDR3
TRGGLD

SEQ ID NO: 148 - protein, 6 aa, synthetic construct
  REGION 1..6: note = mAb24-LCDR1
QDVSTA

SEQ ID NO: 149 - protein, 6 aa, synthetic construct
  REGION 1..6: note = mAb25-LCDR1
QSVSNE

SEQ ID NO: 150 - protein, 8 aa, synthetic construct
  REGION 1..8: note = mAb27-HCDR1
GYTFTTYW

SEQ ID NO: 151 - protein, 5 aa, synthetic construct
  REGION 1..5: note = mAb27-HCDR3
TRAFD

SEQ ID NO: 152 - protein, 8 aa, synthetic construct
  REGION 1..8: note = mAb28-HCDR1
GYTFTSYD

SEQ ID NO: 153 - protein, 8 aa, synthetic construct
  REGION 1..8: note = mAb28-HCDR2
IYPREIIP

SEQ ID NO: 154 - protein, 12 aa, synthetic construct
  REGION 1..12: note = mAb28-HCDR3
AMWGSHLYALDY

SEQ ID NO: 155 - protein, 7 aa, synthetic construct
  REGION 1..7: note = mAb28,29-LCDR1
SSISSSN

SEQ ID NO: 156 - skipped

SEQ ID NO: 157 - protein, 10 aa, synthetic construct
  REGION 1..10: note = mAb28-LCDR3
QQWSSYPHIT

SEQ ID NO: 158 - protein, 11 aa, synthetic construct
  REGION 1..11: note = mAb29-HCDR3
TRGPDGYFEDY

SEQ ID NO: 159 - protein, 9 aa, synthetic construct
  REGION 1..9: note = mAb29-LCDR3
QQWSSYPYT

SEQ ID NO: 160 - protein, 25 aa, synthetic construct [sequence omitted]

SEQ ID NO: 161 - protein, 17 aa, synthetic construct
  REGION 1..17: note = mAb1-HFR2
MHWVKQTPRQGLEWIGS

SEQ ID NO: 162 - protein, 38 aa, synthetic construct
  REGION 1..38: note = mAb1-HFR3
SYNQKFRDKATLTVDKSSSTAYMQLSSLTSEDSAVYFC

SEQ ID NO: 163 - protein, 11 aa, synthetic construct
  REGION 1..11: note = mAb1,2,7,13,18,19,20,23,24,29-HFR4
WGQGTTLTVSS

SEQ ID NO: 164 - protein, 26 aa, synthetic construct
  REGION 1..26: note = mAb1-LFR1
SIVMTQTPKFLPISAGDRVTMTCKAS

SEQ ID NO: 165 - protein, 17 aa, synthetic construct
  REGION 1..17: note = mAb1,18,24,25-LFR2
VAWYQQKPGQSPKLLIY

SEQ ID NO: 166 - protein, 36 aa, synthetic construct
  REGION 1..36: note = mAb1,25-LFR3
NRYTGVPDRFTGSGSGTDFTFTISSVQVEDLAVYFC

SEQ ID NO: 167 - protein, 10 aa, synthetic construct
  REGION 1..10: note = mAb1,7,13,20-LFR4
FGSGTKLEIK

SEQ ID NO: 168 - protein, 25 aa, synthetic construct
  REGION 1..25: note = mAb2-HFR1
EVQLQQSGTVLARPGASVKMSCRAS

SEQ ID NO: 169 - protein, 17 aa, synthetic construct
  REGION 1..17: note = mAb2,5,6,7,9,10,13,14,16,17,21,22,25,26,27-HFR2
MHWVKQRPGQGLEWIGA

SEQ ID NO: 170 - protein, 38 aa, synthetic construct
  REGION 1..38: note = mAb2-HFR3
DYNQKFKGKAKLTAVTSASSAYMELSSLTNEDSAVYYC

SEQ ID NO: 171 - protein, 26 aa, synthetic construct
  REGION 1..26: note = mAb2,16,17,20,22,26,27-LFR1
DIVMTQSQKFMSTSVGDRVSVTCKAS

SEQ ID NO: 172 - protein, 17 aa, synthetic construct
  REGION 1..17: note = mAb2-LFR2
VAWYQKKPGQSPKVVIY

SEQ ID NO: 173 - protein, 36 aa, synthetic construct
  REGION 1..36: note = mAb2-LFR3
TRYTGVPDRFTGSGLGTDFTLTISNVQSEDLAEYFC

SEQ ID NO: 174 - protein, 10 aa, synthetic construct
  REGION 1..10: note = mAb2,4,5,6,8,10,11,12,14,16,17,18,21,22,23,24,25,26 ,27,29-LFR4
FGGGTKLEIK

SEQ ID NO: 175 - protein, 25 aa, synthetic construct
  REGION 1..25: note = mAb3-HFR1
DVQLQESGPGLVKPSQSLSLTCSVT

SEQ ID NO: 176 - protein, 17 aa, synthetic construct
  REGION 1..17: note = mAb3-HFR2
WNWIRQFPGNKLEWMGY

SEQ ID NO: 177 - protein, 38 aa, synthetic construct
  REGION 1..38: note = mAb3-HFR3
NYNPSLKNRISITRDTSKNQFFLRLNSVTTEDTATYYC

SEQ ID NO: 178 - protein, 11 aa, synthetic construct
  REGION 1..11: note = mAb3,4,8,11,14,17,28-HFR4
WGQGTSVTVSS

SEQ ID NO: 179 - protein, 26 aa, synthetic construct
  REGION 1..26: note = mAb3-LFR1
DIQMTQTTSSLSASLGDRVTISCRAS

SEQ ID NO: 180 - protein, 17 aa, synthetic construct
  REGION 1..17: note = mAb3-LFR2
LNWYQQKPDGTVKLLIY

SEQ ID NO: 181 - protein, 36 aa, synthetic construct
  REGION 1..36: note = mAb3-LFR3
RLHSGVPLRFSGSGSGTDFSLTISNLEPEDIATYYC

SEQ ID NO: 182 - protein, 10 aa, synthetic construct
  REGION 1..10: note = mAb3-LFR4
FGGGTKLELK

SEQ ID NO: 183 - protein, 17 aa, synthetic construct
  REGION 1..17: note = mAb4,18-HFR2
MHWVKQTPRQGLEWIGA

SEQ ID NO: 184 - protein, 38 aa, synthetic construct [sequence omitted]

SEQ ID NO: 185 - protein, 26 aa, synthetic construct
  REGION 1..26: note = mAb4-LFR1
EIQMTQSPSSMSASLGDRITITCQAT

SEQ ID NO: 186 - protein, 17 aa, synthetic construct
  REGION 1..17: note = mAb4-LFR2
LNWYQQKPGKPPSFLIY

SEQ ID NO: 187 - protein, 36 aa, synthetic construct
  REGION 1..36: note = mAb4-LFR3
ELAEGVPSRFSGSGSGSDYSLTISNLESEDFADYYC

SEQ ID NO: 188 - protein, 25 aa, synthetic construct
  REGION 1..25: note = mAb5,20,21-HFR1
EVQLQQSGTVLARPGASVKMSCKTS

SEQ ID NO: 189 - protein, 38 aa, synthetic construct
  REGION 1..38: note = mAb5-HFR3
SYNQKFKDKAKVTAVTSASTAYMEVSSLTNEDSAVYYC

SEQ ID NO: 190 - protein, 11 aa, synthetic construct
  REGION 1..11: note = mAb5,16,22,26,27-HFR4
WGQGTLVTVSA

SEQ ID NO: 191 - protein, 26 aa, synthetic construct
  REGION 1..26: note = mAb5-LFR1
DIVMTQSQKFMSTLVGDRVSVTCKAS

SEQ ID NO: 192 - protein, 17 aa, synthetic construct
  REGION 1..17: note = mAb5-LFR2
VAWFQKKPGQSPKALIY

SEQ ID NO: 193 - protein, 36 aa, synthetic construct
  REGION 1..36: note = mAb5-LFR3
TRYTGVPDHFTGSGSGTDFTLTISNVQSEDLAEYFC

SEQ ID NO: 194 - protein, 25 aa, synthetic construct
  REGION 1..25: note = mAb6,7,10,13,16,17,19,23,26-HFR1
EVQLQQSGTVLARPGASVKMSCKAS

SEQ ID NO: 195 - protein, 38 aa, synthetic construct
  REGION 1..38: note = mAb6-HFR3
NYNQKFKGKAKLTAVTSASTAYMDLSSLTNEDSAVYYC

SEQ ID NO: 196 - protein, 11 aa, synthetic construct
  REGION 1..11: note = mAb6-HFR4
WGQGTTLTVSA

SEQ ID NO: 197 - protein, 26 aa, synthetic construct
  REGION 1..26: note = mAb6-LFR1
ENVLTQSPAIMSATLGEKVTMSCRAS

SEQ ID NO: 198 - protein, 17 aa, synthetic construct
  REGION 1..17: note = mAb6-LFR2
MYWSQQKSGASPKLWIY

SEQ ID NO: 199 - protein, 36 aa, synthetic construct
  REGION 1..36: note = mAb6-LFR3, mAb23-LFR3
NLASGVPARFSGSGSGTSYSLTISSVEAEDAATYYC

SEQ ID NO: 200 - protein, 38 aa, synthetic construct
  REGION 1..38: note = mAb7-HFR3, mAb26-HFR3
SYNQKFKGKAKLTAVTSASTAYMELSSLTNEDSAVYYC

SEQ ID NO: 201 - protein, 26 aa, synthetic construct
  REGION 1..26: note = mAb7,14,15-LFR1
QIVLTQSPAIMSASPGEKVTITCSAS

SEQ ID NO: 202 - protein, 17 aa, synthetic construct
  REGION 1..17: note = mAb7-LFR2
MHWLQQKPGTSPKLWIY

SEQ ID NO: 203 - protein, 36 aa, synthetic construct
  REGION 1..36: note = mAb7-LFR3, mAb9-LFR3
NLASGVPARFSGSGSGTSYSLTISRMEAEDAATYYC

SEQ ID NO: 204 - protein, 25 aa, synthetic construct
  REGION 1..25: note = mAb8-HFR1, mAb11-HFR1
QAYLQQSGAELVRSGASVKMSCKAS

SEQ ID NO: 205 - protein, 17 aa, synthetic construct
  REGION 1..17: note = mAb8-HFR2
MHWVKQTPGQGLEWIGY

SEQ ID NO: 206 - protein, 38 aa, synthetic construct
  REGION 1..38: note = mAb8-HFR3, mAb11-HFR3
NYNQKFKGKATLTADTSSSTAYMQISSLTSEDSAVYFC

SEQ ID NO: 207 - protein, 26 aa, synthetic construct
  REGION 1..26: note = mAb8-LFR1
DIVMTQSHKFMSTSVGDRVNITCKAS

SEQ ID NO: 208 - protein, 17 aa, synthetic construct
  REGION 1..17: note = mAb8-LFR2
VAWYQHKPGQSPKLLIY

SEQ ID NO: 209 - protein, 36 aa, synthetic construct
  REGION 1..36: note = mAb8,11,18,24-LFR3
YRYTGVPDRFTGSGSGTDFTFTISSVQAEDLAVYYC

SEQ ID NO: 210 - protein, 25 aa, synthetic construct
  REGION 1..25: note = mAb9,15-HFR1
EVQLQQSGTLLARPGASVKMSCKAS

SEQ ID NO: 211 - protein, 38 aa, synthetic construct
  REGION 1..38: note = mAb9-HFR3, mAb15-HFR3
TYNQKFKGKARLTAVTSASTAYMELSSLTNEDSAVYYC

SEQ ID NO: 212 - protein, 11 aa, synthetic construct
  REGION 1..11: note = mAb9-HFR4, mAb15-HFR4
WGQGTSVSVSS

SEQ ID NO: 213 - protein, 26 aa, synthetic construct
  REGION 1..26: note = mAb9-LFR1
QIVLTQSPAIMSASPGEKVTMTCSAS

SEQ ID NO: 214 - protein, 17 aa, synthetic construct
  REGION 1..17: note = mAb9-LFR2, mAb15-LFR2
MYWLQQKPGTSPKLWIY

SEQ ID NO: 215 - protein, 10 aa, synthetic construct
  REGION 1..10: note = mAb9-LFR4, mAb15-LFR4
FGGGANLEIK

SEQ ID NO: 216 - protein, 38 aa, synthetic construct
  REGION 1..38: note = mAb10-HFR3
SYNQKFKDKAKLTAVTSTSTAYMELSSLTNEDSAVYYC

SEQ ID NO: 217 - protein, 11 aa, synthetic construct
  REGION 1..11: note = mAb10-HFR4
WGAGTTVTVSS

SEQ ID NO: 218 - protein, 26 aa, synthetic construct
  REGION 1..26: note = mAb10-LFR1
QIVLTQSPALMSASPGEKVTMTCSAS

SEQ ID NO: 219 - protein, 17 aa, synthetic construct
  REGION 1..17: note = mAb10,13-LFR2
MYWYQQKPRSSPKPWIY

SEQ ID NO: 220 - protein, 36 aa, synthetic construct
  REGION 1..36: note = mAb10-LFR3, mAb13-LFR3
NLASGVPARFSGSGSGTSYSLTISSMEAEDAATYYC

SEQ ID NO: 221 - protein, 17 aa, synthetic construct
  REGION 1..17: note = mAb11-HFR2
MHWVKRTPGQGLEWIGY

SEQ ID NO: 222 - protein, 26 aa, synthetic construct
  REGION 1..26: note = mAb11,18,24-LFR1
DIVMTQSHKFMSTSVGDRVSITCKAS

SEQ ID NO: 223 - protein, 17 aa, synthetic construct
  REGION 1..17: note = mAb11-LFR2
VAWYQQSPGQSPKLLIY

SEQ ID NO: 224 - protein, 25 aa, synthetic construct [sequence omitted]

SEQ ID NO: 225 - protein, 17 aa, synthetic construct
  REGION 1..17: note = mAb12-HFR2
MHWVKQRPGQGLEWIGV

SEQ ID NO: 226 - protein, 38 aa, synthetic construct
  REGION 1..38: note = mAb12-HFR3
SYNQKFKGKATLTVDTSSSTAYMQLSSLTSEDSAVYYC

SEQ ID NO: 227 - protein, 11 aa, synthetic construct
  REGION 1..11: note = mAb12-HFR4
WGQGTTLTVPS

SEQ ID NO: 228 - protein, 26 aa, synthetic construct
  REGION 1..26: note = mAb12-LFR1
DIVLTQSPASLAVSLGQRATISCRAS

SEQ ID NO: 229 - protein, 17 aa, synthetic construct
  REGION 1..17: note = mAb12-LFR2
MNWFQQKPGQPPKLLIY

SEQ ID NO: 230 - protein, 36 aa, synthetic construct
  REGION 1..36: note = mAb12-LFR3
NQGSGVPARFSGSGSGTDFSLNIHPMEEDDTAMYFC

SEQ ID NO: 231 - protein, 38 aa, synthetic construct
  REGION 1..38: note = mAb13-HFR3
DYNQKFKGKAKLTAVTSTSTAYMELSSLTNEDSAVYYC

SEQ ID NO: 232 - protein, 26 aa, synthetic construct
  REGION 1..26: note = mAb13-LFR1
QIVLTQSPAVMSASPGEKVTMTCSAS

SEQ ID NO: 233 - protein, 25 aa, synthetic construct
  REGION 1..25: note = mAb14-HFR1
EVHLQQSGTVLARPGASVKMSCKAS

SEQ ID NO: 234 - protein, 38 aa, synthetic construct
  REGION 1..38: note = mAb14-HFR3
SYNQKFKAKAKLTAVTSASTAYMELSSLTNEDSAVYYC

SEQ ID NO: 235 - protein, 17 aa, synthetic construct
  REGION 1..17: note = mAb14-LFR2
MYWVQQKPGTSPKLWIY

SEQ ID NO: 236 - protein, 36 aa, synthetic construct
  REGION 1..36: note = mAb14-LFR3
KLASGVPARFSGSGSGTSYSLTISRMEAEDAATYYC

SEQ ID NO: 237 - protein, 17 aa, synthetic construct
  REGION 1..17: note = mAb15-HFR2
MHWVKQRPGQNLEWIGA

SEQ ID NO: 238 - protein, 36 aa, synthetic construct
  REGION 1..36: note = mAb15-LFR3
NLASGVPDRFSGSGSGTSYSLTISRMEAEDAATYYC

SEQ ID NO: 239 - protein, 38 aa, synthetic construct
  REGION 1..38: note = mAb16-HFR3, mAb25-HFR3
SYNQKFKGKAKLTAVTSASTAYMDLSSLTNEDSAVYYC

SEQ ID NO: 240 - protein, 17 aa, synthetic construct
  REGION 1..17: note = mAb16,22,26-LFR2
VAWYQKKPGQSPKALIY

SEQ ID NO: 241 - protein, 36 aa, synthetic construct
  REGION 1..36: note = mAb16,22,26-LFR3
TRYTGVPDRFTGSGSGTDFTLTISNVQSEDLAEYFC

SEQ ID NO: 242 - protein, 38 aa, synthetic construct
  REGION 1..38: note = mAb17-HFR3
DYNQKFKGKAKLTAVTSASTAYMELSSLTNEDSAVYYC

SEQ ID NO: 243 - protein, 17 aa, synthetic construct
  REGION 1..17: note = mAb17,20,27-LFR2
VAWYQQKPGQSPKALIY

SEQ ID NO: 244 - protein, 36 aa, synthetic construct
  REGION 1..36: note = mAb17-LFR3
YRYSGVPDRFTGSGSGTDFTLTISNVQSEDLADYFC

SEQ ID NO: 245 - protein, 17 aa, synthetic construct
  REGION 1..17: note = mAb19-HFR2
MHWIIQRPGQGLEWIGS

SEQ ID NO: 246 - protein, 38 aa, synthetic construct
  REGION 1..38: note = mAb19-HFR3
SYNQKFMDKAKLTAVTSASTAYLELSSLTNEDSAVYYC

SEQ ID NO: 247 - protein, 26 aa, synthetic construct
  REGION 1..26: note = mAb19-LFR1
DIQMTQSPASLSASVGETVTITCRAS

SEQ ID NO: 248 - protein, 17 aa, synthetic construct
  REGION 1..17: note = mAb19-LFR2
LAWYQQKEGKSPQLLVY

SEQ ID NO: 249 - protein, 36 aa, synthetic construct
  REGION 1..36: note = mAb19-LFR3
KLADGVPSRFSGSGSGTQFSLKIKSLQPEDFGSYYC

SEQ ID NO: 250 - protein, 10 aa, synthetic construct
  REGION 1..10: note = mAb19-LFR4
FGGGTKVEIK

SEQ ID NO: 251 - protein, 17 aa, synthetic construct
  REGION 1..17: note = mAb20-HFR2
IHWIKQRPGQGLEYIGA

SEQ ID NO: 252 - protein, 38 aa, synthetic construct
  REGION 1..38: note = mAb20-HFR3
TYNQKFKGKAKLTAVTSASTAYMDLNSLTDEDSAVYYC

SEQ ID NO: 253 - protein, 36 aa, synthetic construct
  REGION 1..36: note = mAb20-LFR3
YRYSGVPARFTGSGSGTDFTLTISNVQSEDLAEYYC

SEQ ID NO: 254 - protein, 38 aa, synthetic construct
  REGION 1..38: note = mAb21-HFR3, mAb29-HFR3
NYNQNFKGKAKLTAVTSASTAYMELSSLTNEDSAVYYC

SEQ ID NO: 255 - protein, 11 aa, synthetic construct
  REGION 1..11: note = mAb21-HFR4
WGTGTTVTISS

SEQ ID NO: 256 - protein, 26 aa, synthetic construct
  REGION 1..26: note = mAb21-LFR1
DIVMTQSPASLSASVGETVTMTCRAS

SEQ ID NO: 257 - protein, 17 aa, synthetic construct
  REGION 1..17: note = mAb21-LFR2
LAWYQQKQGKSPQLLVY

SEQ ID NO: 258 - protein, 36 aa, synthetic construct
  REGION 1..36: note = mAb21-LFR3
NLADGVPSRFSGSGSGTQFSLKINSLQPEDFGSYYC

SEQ ID NO: 259 - protein, 25 aa, synthetic construct
  REGION 1..25: note = mAb22-HFR1
EVQLQQSGAELARPGASVKMSCKTS

SEQ ID NO: 260 - protein, 38 aa, synthetic construct
  REGION 1..38: note = mAb22-HFR3
DYNQKFMDKAKLTSVTSASTAYMELSSLTDEDSAVYFC

SEQ ID NO: 261 - protein, 17 aa, synthetic construct
  REGION 1..17: note = mAb23-HFR2
MHWVKQRPGQGLEWIGS

SEQ ID NO: 262 - protein, 38 aa, synthetic construct
  REGION 1..38: note = mAb23-HFR3
NYNQKFKGKAKLTAVTSASTAYLDLSSLTNEDSAVYYC

SEQ ID NO: 263 - protein, 26 aa, synthetic construct
  REGION 1..26: note = mAb23-LFR1
DIVLTQSPAIMSATLGEKVTMSCRAS

SEQ ID NO: 264 - protein, 17 aa, synthetic construct
  REGION 1..17: note = mAb23-LFR2
MYWYQQKSGASPKLWIY

SEQ ID NO: 265 - protein, 25 aa, synthetic construct
  REGION 1..25: note = mAb24-HFR1
EVQLQQSGAVLARPGTSVKMSCKAS

SEQ ID NO: 266 - protein, 17 aa, synthetic construct
  REGION 1..17: note = mAb24-HFR2
IHWIKQRPGQGLEWLGA

SEQ ID NO: 267 - protein, 38 aa, synthetic construct
  REGION 1..38: note = mAb24-HFR3
DYNQKVRAKAKLTAVTSASTAYMELSSLTNEDSAVYYC

SEQ ID NO: 268 - protein, 25 aa, synthetic construct
  REGION 1..25: note = mAb25-HFR1
EVQLQQSGPELVKPGASVKLSCKAS

SEQ ID NO: 269 - protein, 11 aa, synthetic construct
  REGION 1..11: note = mAb25-HFR4
WGQGTLVTVSS

SEQ ID NO: 270 - protein, 26 aa, synthetic construct
  REGION 1..26: note = mAb25-LFR1
DIVMTQSPKFLPVTAEDRVTITCKAS

SEQ ID NO: 271 - protein, 25 aa, synthetic construct
  REGION 1..25: note = mAb27-HFR1
EVQLQQSGPELVKPGASVKISCKAS

SEQ ID NO: 272 - protein, 38 aa, synthetic construct
  REGION 1..38: note = mAb27-HFR3
SYNQKFKDKAKLTAVTSANTAYMELSSLTNEDSAVYYC

SEQ ID NO: 273 - protein, 36 aa, synthetic construct
  REGION 1..36: note = mAb27-LFR3
YRYSGVPDRFTGSGSGTDFTLTISSVQAEDLAVYYC

SEQ ID NO: 274 - protein, 25 aa, synthetic construct
  REGION 1..25: note = mAb28-HFR1
QVQLQQSGSELVKPGASVKLSCKAS

SEQ ID NO: 275 - protein, 17 aa, synthetic construct
  REGION 1..17: note = mAb28-HFR2
INWVKQRPGQGLEWIGW

SEQ ID NO: 276 - protein, 38 aa, synthetic construct
  REGION 1..38: note = mAb28-HFR3
KYNEKFKGKATLTVDTSSSTAYMELHSLTSEDSAVYFC

SEQ ID NO: 277 - protein, 26 aa, synthetic construct
  REGION 1..26: note = mAb28-LFR1
DIELTQSPALMATSPGEKVTITCSVS

SEQ ID NO: 278 - protein, 17 aa, synthetic construct
  REGION 1..17: note = mAb28-LFR2, mAb29-LFR2
LHWYQQKSGTSPKLWIY

SEQ ID NO: 279 - protein, 36 aa, synthetic construct
  REGION 1..36: note = mAb28-LFR3, mAb29-LFR3
NLASGVPVRFSGSGSGTSYSLTISSMEAEDAATYYC

SEQ ID NO: 280 - protein, 10 aa, synthetic construct
  REGION 1..10: note = mAb28-LFR4
FGAGTKLELK

SEQ ID NO: 281 - protein, 25 aa, synthetic construct
  REGION 1..25: note = mAb29-HFR1
QVQLQQSGTVLARPGASVKMSCKAS

SEQ ID NO: 282 - protein, 17 aa, synthetic construct
  REGION 1..17: note = mAb29-HFR2
MHWLKQRPGQGLEWIGA

SEQ ID NO: 283 - protein, 26 aa, synthetic construct
  REGION 1..26: note = mAb29-LFR1
DIVLTQSPASLAVSLGQKVTITCSVS

SEQ ID NO: 284 - protein, 25 aa, synthetic construct
  REGION 1..25: note = mAb18-HFR1
QVHVKQSGAELVRPGASVKMSCKAS

SEQ ID NO: 285 - protein, 113 aa, synthetic construct
  REGION 1..113: note = humAb1-VH
QVQLVQSGAEVKKPGASVKVSCKASGYTFTSYNMHWVRQAPGQGLEWMGSIYPENGDTSYNQKFQDRVTLTRDTSTSTVYMELSSLRSEDTAVYYCARPFDYWGQGTLVTVSS

SEQ ID NO: 286 - protein, 107 aa, synthetic construct
  REGION 1..107: note = humAb1-VL, humAb5- VL, humAb6-VL
DIQMTQSPSSLSASVGDRVTITCQASQSVGNNLAWYQQKPGKAPKLLIYYASNLYTGVPSRFSGSGSGTDFTFTISSLQPEDIATYYCQQFYSSPYTFGQGTKLEIK

SEQ ID NO: 287 - protein, 113 aa, synthetic construct
  REGION 1..113: note = humAb2-VH, humAb3-VH
QAQLVQSGAEVKKPGASVKVSCKASGYTFTSYNMHWVRQAPGQGLEWMGSIYPENGDTSYNQKFQDRVTLTRDTSTSTVYMELSSLRSEDTAVYYCARPFDYWGQGTLVTVSS

SEQ ID NO: 288 - protein, 107 aa, synthetic construct
  REGION 1..107: note = humAb2-VL, humAb4- VL
DIVMTQSPSSLSASVGDRVTITCQASQSVGNNVAWYQQKPGKAPKLLIYYASNLYTGVPSRFSGSGSGTDFTFTISSLQPEDIATYYCQQFYSSPYTFGQGTKLEIK

SEQ ID NO: 289 - protein, 107 aa, synthetic construct
  REGION 1..107: note = humAb3-VL
DIQMTQSPSSLSASVGDRVTITCQASQSVGNNVAWYQQKPGKAPKLLIYYASNLYTGVPSRFSGSGSGTDFTFTISSLQPEDIATYYCQQFYSSPYTFGQGTKLEIK

SEQ ID NO: 290 - protein, 113 aa, synthetic construct
  REGION 1..113: note = humAb4-VH, humAb5-VH
QVQLVQSGAEVKKPGASVKVSCKASGYTFTSYNMHWVRQAPGQGLEWMGSIYPENGDTSYNQKFQGRVTLTRDTSTSTVYMELSSLRSEDTAVYYCARPFDYWGQGTLVTVSS

SEQ ID NO: 291 - protein, 113 aa, synthetic construct
  REGION 1..113: note = humAb6-VH
QAQLVQSGAEVKKPGASVKVSCKASGYTFTSYNMHWVRQAPGQGLEWMGSIYPENGDTSYNQKFQGRVTLTRDTSTSTVYMELSSLRSEDTAVYYCARPFDYWGQGTLVTVSS

SEQ ID NO: 292 - protein, 442 aa, synthetic construct
  REGION 1..442: note = humAbA1-H
QVQLVQSGAEVKKPGASVKVSCKASGYTFTSYNMHWVRQAPGQGLEWMGSIYPENGDTSYNQKFQDRVTLTRDTSTSTVYMELSSLRSEDTAVYYCARPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 293 - protein, 214 aa, synthetic construct
  REGION 1..214: note = humAbA1-L, humAbA2-L
DIQMTQSPSSLSASVGDRVTITCQASQSVGNNLAWYQQKPGKAPKLLIYYASNLYTGVPSRFSGSGSGTDFTFTISSLQPEDIATYYCQQFYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 294 - protein, 442 aa, synthetic construct
  REGION 1..442: note = humAbA2-H
QVQLVQSGAEVKKPGASVKVSCKASGYTFTSYNMHWVRQAPGQGLEWMGSIYPENGDTSYNQKFQDRVTLTRDTSTSTVYMELSSLRSEDTAVYYCARPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPG

SEQ ID NO: 295 - protein, 25 aa, synthetic construct
  REGION 1..25: note = humAb2-HFR1, humAb3-HFR1, humAb6-HFR1
QAQLVQSGAEVKKPGASVKVSCKAS

SEQ ID NO: 296 - protein, 17 aa, synthetic construct
  REGION 1..17: note = humAb1-humAb6-HFR2, humAbA1-HFR2, humAbA2-HFR2
MHWVRQAPGQGLEWMGS

SEQ ID NO: 297 - protein, 38 aa, synthetic construct
  REGION 1..38: note = humAb1-humAb3-HFR3, humAbA1-HFR3, humAbA2-HFR3
SYNQKFQDRVTLTRDTSTSTVYMELSSLRSEDTAVYYC

SEQ ID NO: 298 - protein, 21 aa, synthetic construct
  REGION 1..21: note = humAb1-humAb6-HFR4, humAbA1-HFR4, humAbA2-HFR4
WGQGTLVTVSSASTKGPSVFP

SEQ ID NO: 299 - protein, 26 aa, synthetic construct
  REGION 1..26: note = humAb1,3,5,6-LFR1, humAbA1-LFR1, humAbA2-LFR1
DIQMTQSPSSLSASVGDRVTITCQAS

SEQ ID NO: 300 - protein, 17 aa, synthetic construct
  REGION 1..17: note = humAb1,5,6-LFR2, humAbA1,2-LFR2
LAWYQQKPGKAPKLLIY

SEQ ID NO: 301 - protein, 36 aa, synthetic construct
  REGION 1..36: note = humAb1,2,3,4,5,6-LFR3, humAbA1-LFR3, humAbA2-LFR3
NLYTGVPSRFSGSGSGTDFTFTISSLQPEDIATYYC

SEQ ID NO: 302 - protein, 22 aa, synthetic construct
  REGION 1..22: note = humAb1,2,3,4,5,6-LFR4, humAbA1-LFR4, humAbA2-LFR4
FGQGTKLEIKRTVAAPSVFIFP

SEQ ID NO: 303 - protein, 26 aa, synthetic construct
  REGION 1..26: note = humAb2-LFR1, humAb4-LFR1
DIVMTQSPSSLSASVGDRVTITCQAS

SEQ ID NO: 304 - protein, 17 aa, synthetic construct
  REGION 1..17: note = humAb2,3,4-LFR2
VAWYQQKPGKAPKLLIY

SEQ ID NO: 305 - protein, 25 aa, synthetic construct [sequence omitted]

SEQ ID NO: 306 - protein, 38 aa, synthetic construct
  REGION 1..38: note = humAb4,5,6-HFR3
SYNQKFQGRVTLTRDTSTSTVYMELSSLRSEDTAVYYC

SEQ ID NO: 307 - protein, 98 aa, synthetic construct
  REGION 1..98: note = HumAbA1 CH1, HumAbA2 CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 308 - protein, 15 aa, synthetic construct
  REGION 1..15: note = HumAbA1 Hinge, HumAbA2 Hinge
EPKSCDKTHTCPPCP

SEQ ID NO: 309 - protein, 110 aa, synthetic construct
  REGION 1..110: note = HumAbA1 CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 310 - protein, 106 aa, synthetic construct
  REGION 1..106: note = HumAbA1 CH3
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 311 - protein, 107 aa, synthetic construct [sequence omitted]

SEQ ID NO: 312 - protein, 110 aa, synthetic construct
  REGION 1..110: note = HumAbA2 CH2
APELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAK

SEQ ID NO: 313 - protein, 106 aa, synthetic construct
  REGION 1..106: note = HumAbA2 CH3
GQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPG